(19) United States
(12) Patent Application Publication
(10) Pub. No.: US 2025/0381183 A1
(43) Pub. Date: Dec. 18, 2025

(54) TARGETING S100A9-ALDH1A1-RETINOIC ACID SIGNALING TO SUPPRESS BRAIN RELAPSE IN EGFR-MUTANT LUNG CANCER

(71) Applicant: The Trustees of Columbia University in the City of New York, New York, NY (US)

(72) Inventors: Anup Kumar BISWAS, New York, NY (US); Swarnali ACHARYYA, New York, NY (US)

(21) Appl. No.: 18/783,386

(22) Filed: Jul. 24, 2024

Related U.S. Application Data
(63) Continuation of application No. PCT/US2023/061190, filed on Jan. 24, 2023.
(60) Provisional application No. 63/302,410, filed on Jan. 24, 2022.

(51) Int. Cl. A61K 31/506; A61K 31/382; A61P 35/04; G01N 33/68

(52) U.S. Cl. CPC A61K 31/506; A61K 31/382; A61P 35/04; G01N 33/6893; G01N 2333/4704; G01N 2800/52

(57) ABSTRACT

The epidermal growth factor receptor (EGFR) tyrosine kinase inhibitor (TKI) osimertinib has significantly prolonged progression-free survival (PFS) in EGFR-mutant lung cancer patients, including those with brain metastases. However, osimertinib-treated patients often develop lethal metastatic relapse, often to the brain. The genetic repression of S100A9, ALDH1A1, or RA receptors (RAR) in cancer cells, or treatment with a pan-RAR antagonist, dramatically reduces brain metastasis. S100A9 expression in cancer cells correlates with poor PFS in osimertinib-treated patients, and is identified as a novel, therapeutically targetable S100A9-ALDH1A1-RA axis. A combination of osimertinib and AGN-194310, for example, treats such cancer while avoiding metastatic relapse.

### CROSS-REFERENCE TO RELATED APPLICATION

This application as a continuation of International Application No. PCT/US2023/061190, filed on Jan. 24, 2023, which claims benefit of U.S. Provisional Application No. 63/302,410 filed Jan. 24, 2022, the contents of which are hereby incorporated by this reference.

All patents, patent applications, and publications cited herein are hereby incorporated by reference in their entirety. The disclosures of these publications in their entireties are hereby incorporated by reference into this application.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### GOVERNMENT SUPPORT

This invention was made with government support under grants CA172697 awarded by the National Institutes of Health (NIH) and W81XWH-21-1-0764 awarded by Army/MRMC. The government has certain rights in the invention.

### BACKGROUND

The epidermal growth factor receptor (EGFR) tyrosine kinase inhibitor (TKI) osimertinib has significantly prolonged progression-free survival (PFS) in EGFR-mutant lung cancer patients, including those with brain metastases. However, despite striking initial responses, osimertinib-treated patients eventually develop lethal metastatic relapse, often to the brain. Although osimertinib-refractory brain relapse is a major clinical challenge, its underlying mechanisms remain poorly understood. Using metastatic models of EGFR-mutant lung cancer, we show that cancer cells expressing high intracellular S100A9 escape osimertinib and initiate brain relapses. Mechanistically, S100A9 upregulates ALDH1A1 expression and activates the retinoic acid (RA) signaling pathway in osimertinib-refractory cancer cells. We demonstrate that the genetic repression of S100A9, ALDH1A1, or RA receptors (RAR) in cancer cells, or treatment with a pan-RAR antagonist, dramatically reduces brain metastasis. Importantly, S100A9 expression in cancer cells correlates with poor PFS in osimertinib-treated patients. Our study therefore identifies a novel, therapeutically targetable S100A9-ALDH1A1-RA axis that drives brain relapse.

Treatment with the EGFR-TKI osimertinib prolongs the survival of EGFR-mutant lung cancer patients; however, patients develop metastatic relapses, often to the brain. We identified a novel intracellular S100A9-ALDH1A1-RA signaling pathway that drives lethal brain relapse and can be targeted by pan-RAR antagonists to prevent cancer progression and prolong patient survival.

### SUMMARY

Lung cancer is the leading cause of cancer-related mortality (1). Somatic activating mutations in the epidermal growth factor receptor (EGFR) gene occur in up to 50% of lung cancer patients worldwide (2-4). Targeted therapies with EGFR tyrosine kinase inhibitors (TKIs) have transformed the treatment landscape for EGFR-mutant lung cancer (5-8). EGFR exon-19 (in-frame) deletions (del E746-A750) and exon-21 point mutations (L858R) comprise 90% of the EGFR mutations observed in lung cancer and lead to ligand-independent activation of the EGFR signaling pathway to promote proliferation, migration, and survival of cancer cells (7). While the first- and second-generation EGFR TKIs (erlotinib and afatinib, respectively) showed promising initial responses in EGFR-mutant lung cancer patients, patients eventually acquired therapy resistance (50% of cases with an acquired EGFR T790M mutation) within 9-14 months of treatment, and ultimately developed lethal metastatic relapse (9-11). The subsequent development of third-generation EGFR TKIs led to the discovery of osimertinib (AZD9291), which specifically targets EGFR T790M and the original sensitizing mutations (exon-19 deletion and L858R), while sparing wild-type EGFR and the toxicities associated with its inhibition (12). Remarkably, first-line treatment with osimertinib significantly extended the median progression-free survival (PFS) of EGFR-mutant metastatic lung cancer patients from 10.2 months (involving treatment using earlier-generation EGFR-TKIs) to 18.9 months (13,14). Despite promising initial responses, osimertinib-treated patients eventually develop metastatic relapses and succumb to death (8,15-17). Studies designed to explore osimertinib-resistance mechanisms have found that on-target mutations in the EGFR gene that abrogate the binding of osimertinib to EGFR account for only 6-10% of osimertinib-resistant tumors with first-line treatment (17-19). Strikingly, 50% of osimertinib-refractory relapses instead arise from EGFR-pathway-independent mechanisms, which remain poorly defined (17).

Metastasis to the central nervous system (CNS) is a frequent complication in patients with EGFR-mutant lung cancer (20-22). It is estimated that 25% of patients with EGFR-mutant lung cancer already present with CNS metastases at diagnosis, and the incidence of CNS metastasis increases to 45% at three years post diagnosis and TKI treatment (21,23). Metastasis to the brain portends a poor prognosis, as it is associated with a significant decline in cognitive and motor function, impaired daily functioning, morbidity, and accelerated mortality (21-23). First- and second-generation EGFR-TKIs showed poor blood-brain barrier permeability, and consequently had minimal impact on brain metastases. In contrast, the superior blood-brain barrier permeability of osimertinib led to impressive clinical responses in patients with brain metastasis (23,24). However, responses to osimertinib are not durable, and patients eventually relapse and die with osimertinib-refractory metastatic progression. In particular, CNS progression has been reported in 20% of lung cancer patients treated with osimertinib (25), which adversely affects quality of life and shortens survival. Therefore, an understanding of post-osimertinib CNS relapse mechanisms is critical for improving the clinical management of EGFR-mutant lung cancer patients.

The persistence of residual disease following osimertinib treatment likely contributes to metastatic relapse and presents a clinical challenge for EGFR-mutant lung cancer patients. Tissue microenvironments can provide protective niches for the survival and expansion of residual cancer cells and enable the development of relapsed tumors (26,27). Surprisingly, however, metastatic models are rarely used for studying osimertinib-refractory metastatic relapse mechanisms, which limits the potential for developing more effective therapies. Therefore, to identify mechanisms of osimertinib-refractory relapse in the context of metastatic progression, we utilized mouse models that closely resemble the metastatic progression and osimertinib response observed in human patients.

We therefore generated long-term in-vivo treatment models using osimertinib-sensitive EGFR-mutant human lung cancer cell lines (PC9 and H1650) that metastasize to distant organs, including the brain. These mice show remarkable initial responses to osimertinib that are analogous to human patients, with a long window of progression-free survival, followed by metastatic relapse. To identify the mechanisms underlying osimertinib-refractory relapse using these mouse models, we performed proteomic and transcriptomic profiling of relapsed brain metastatic cells and found that they express high levels of S100A9, a protein that is normally secreted by myeloid cells (28). Of note, clinical studies have found that S100A9 overexpression in lung cancer cells is correlated with poor prognosis in lung cancer patients (29); however, the underlying molecular mechanisms remain unknown. Here, using mouse models and patient samples, we show that intracellular S100A9 expression in EGFR-mutant lung cancer cells drives brain relapse through a previously unknown S100A9-ALDH1A1-RA axis. We demonstrate that genetic inhibition of S100A9, ALDH1A1, or retinoic acid receptors (RAR), or pharmacological inhibition of the RA pathway using pan-RAR antagonists, significantly reduces brain relapse from osimertinib-refractory cancer cells. Our study therefore reveals a novel S100A9-ALDH1A1-RA axis in EGFR-mutant lung cancer cells that drives osimertinib-refractory metastatic brain relapse and identifies a potential vulnerability in lung cancer cells that can be therapeutically targeted to prolong progression-free survival in EGFR-mutant lung cancer patients.

### BRIEF DESCRIPTION OF FIGURES

Fatal brain relapse in osimertinib-treated, PC9-derived, metastatic lung cancer mouse model.

FIG. 1A: Schematic representation of the in-vivo treatment model derived from the PC9-BrM cell line for metastatic EGFR-mutant lung cancer. Luciferase-labeled human EGFR-mutant PC9-BrM cells were injected into the arterial circulation of immunodeficient mice via intracardiac injection to generate metastases, which were detected by bioluminescence imaging. At 25 days post tumor-cell injection, after confirmation of metastatic signal, mice were administered long-term treatment with either vehicle or osimertinib (“Osi”) at 5 mg/kg body weight/day by oral gavage, 5 days per week, until the endpoint indicated in (b-c), which averaged to 8 months post tumor-cell injection. Periods of response to osimertinib and subsequent relapse were detected by bioluminescence imaging.

FIG. 1B: Representative images for longitudinal monitoring of metastatic progression with vehicle (“Veh”) or osimertinib treatment by weekly bioluminescence imaging, with progressive development of osimertinib-refractory brain relapse in mice. Vehicle-treated mice developed bone, brain and lymph node metastases and were euthanized when weight loss was >20% or when the body-conditioning score reached 2. Osimertinib-treated mice were monitored for the emergence and progression of osimertinib-refractory metastasis in the brain and euthanized when either weight loss was >20%, the body-conditioning score reached 2, or mice developed paralysis or seizure-like symptoms due to brain metastasis. Days represent days after initial tumor-cell injection. Photon-flux scales are indicated below the images.

FIG. 1C: Kaplan-Meier plot for brain-metastasis-progression-free survival of mice from the experiment described in panels (a-b). Data were analyzed using the log-rank test: χ2=19.33, d.f.=1, p-value<0.0001, n=10 for vehicle-treated mice and 10 for osimertinib-treated mice.

FIG. 1D: Schematic representation of the experimental design to derive osimertinib-treatment-refractory Tr-BrM cells from relapsed brain metastases from the mice described in panels (a-c) that were injected with PC9-BrM cells and treated long-term with osimertinib.

FIG. 1E: Kaplan-Meier plot for brain-metastasis-progression-free survival of mice injected with PC9-Tr-BrM cells followed by treatment with either vehicle or osimertinib. Data were analyzed using the log-rank test: χ2=1.325, d.f.=1, p-value not significant (ns), n=20 for vehicle-treated mice and 17 for osimertinib-treated mice.

FIG. 1F: Representative images of human cytokeratin 7 (CK7) immunohistochemistry on brain sections from mice injected with either PC9-BrM cells (upper panel) or PC9-Tr-BrM cells (lower panel) and treated with either vehicle (left panel) or osimertinib (right panel). Mice were euthanized at 7 weeks post tumor-cell injection. Scale bars, 200 μm.

FIG. 1G: Quantitative analysis of the percentage of CK7-immunostained brain sections covered by metastasis that are represented in (f). Data are presented as mean values±SEM. P-values were determined by a two-tailed, unpaired Mann-Whitney test: n=10 for vehicle-treated mice bearing PC9-BrM or PC9-Tr-BrM metastases, and n=11 for osimertinib-treated mice bearing PC9-BrM or PC9-Tr-BrM metastases.

### S100A9 is a Key Mediator of Brain Relapse in Osimertinib-Refractory Lung Cancer Cells.

FIG. 2A: Immunoblot analysis for inhibition of the EGFR pathway activation in PC9-BrM and PC9-Tr-BrM cells treated with the indicated doses of osimertinib (“Osi”) and collected at 6 hours post-treatment. Antibodies against phospho-EGFR (Tyr1068), phospho-ERK (Thr202/Tyr204), total-EGFR, total-ERK and □-actin (loading control) were used. Data are representative of three independent experiments.

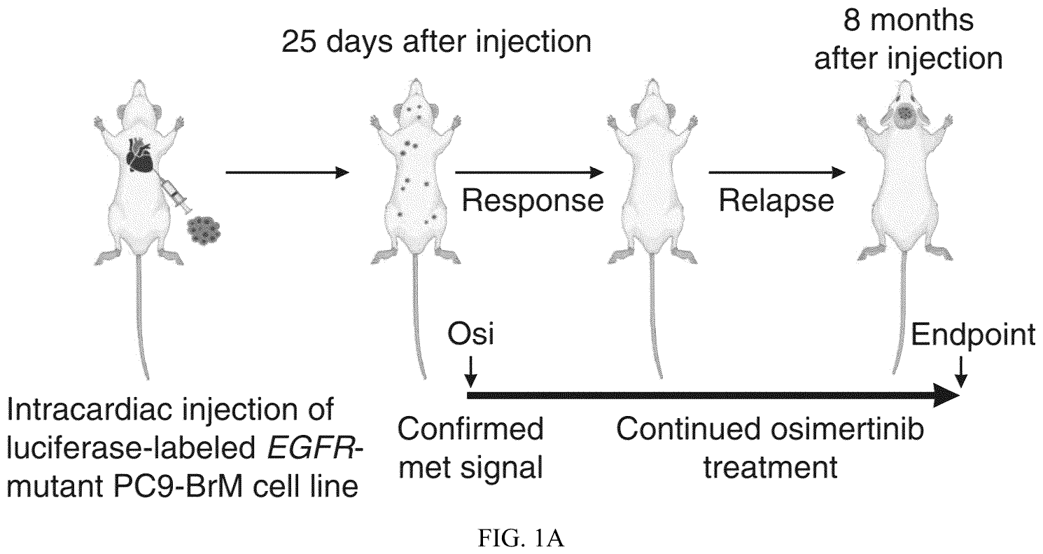

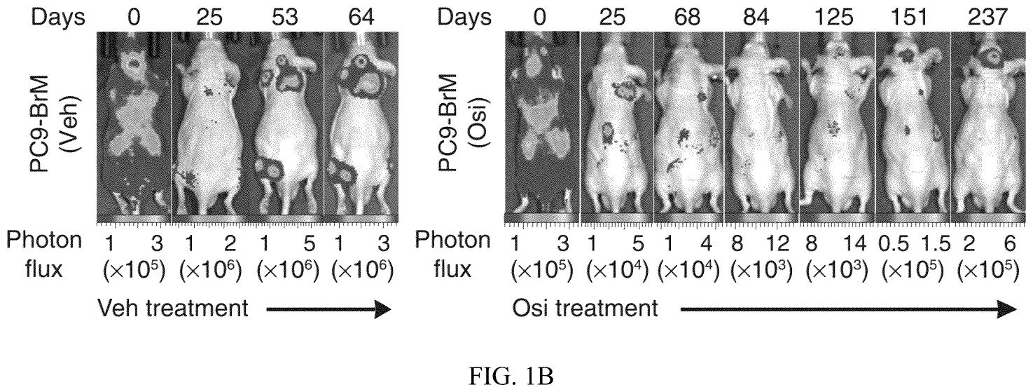

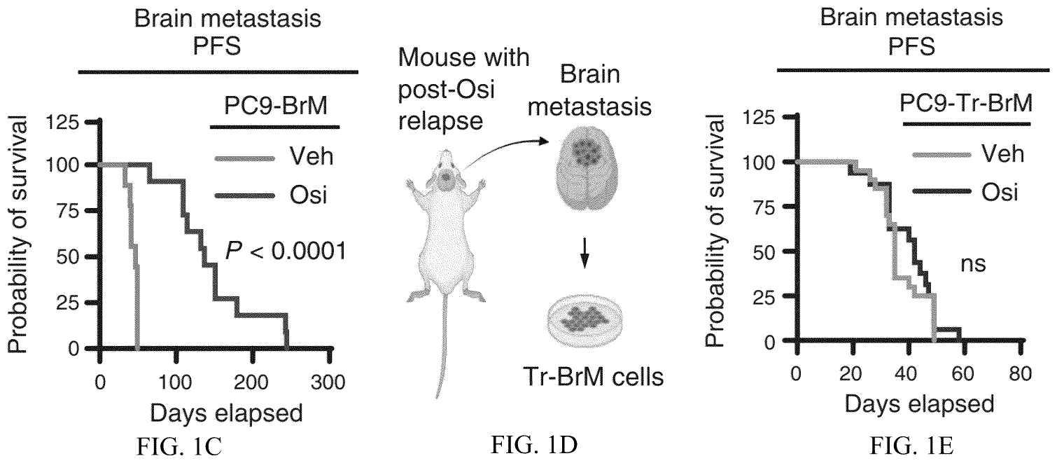

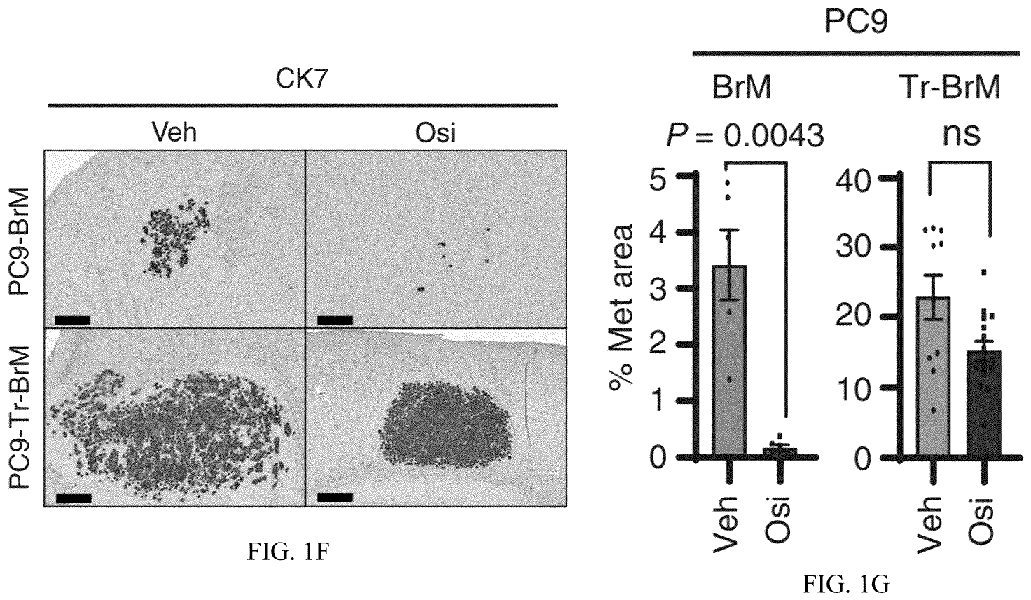

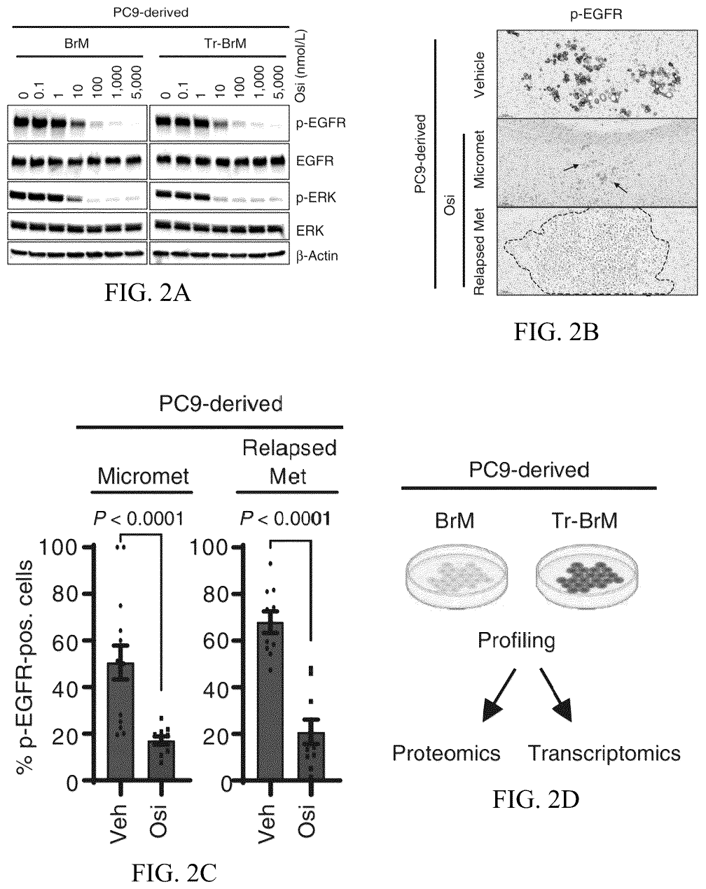

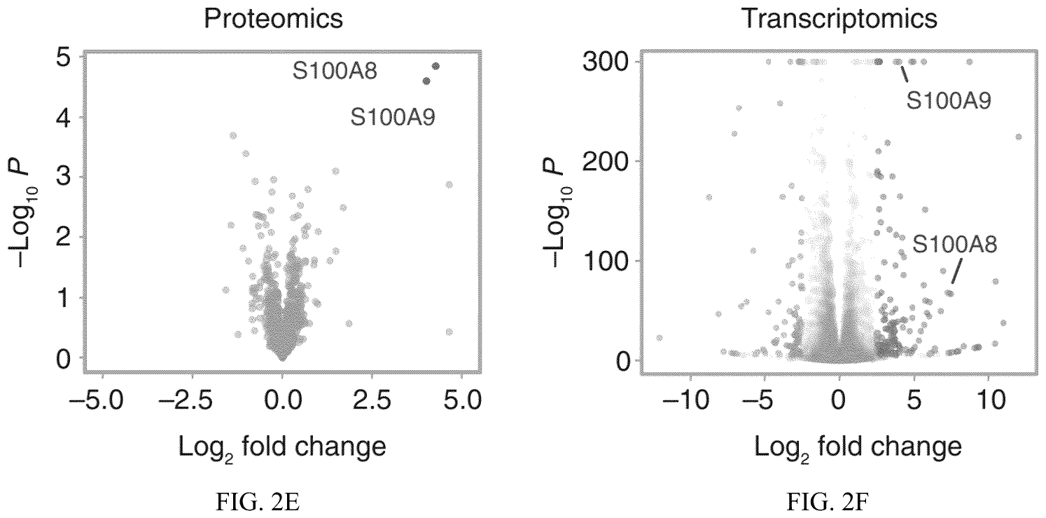

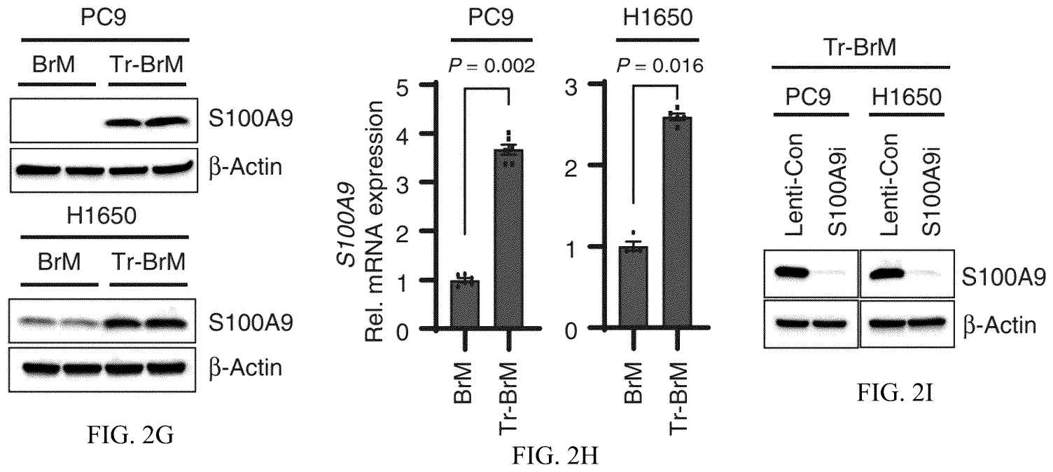

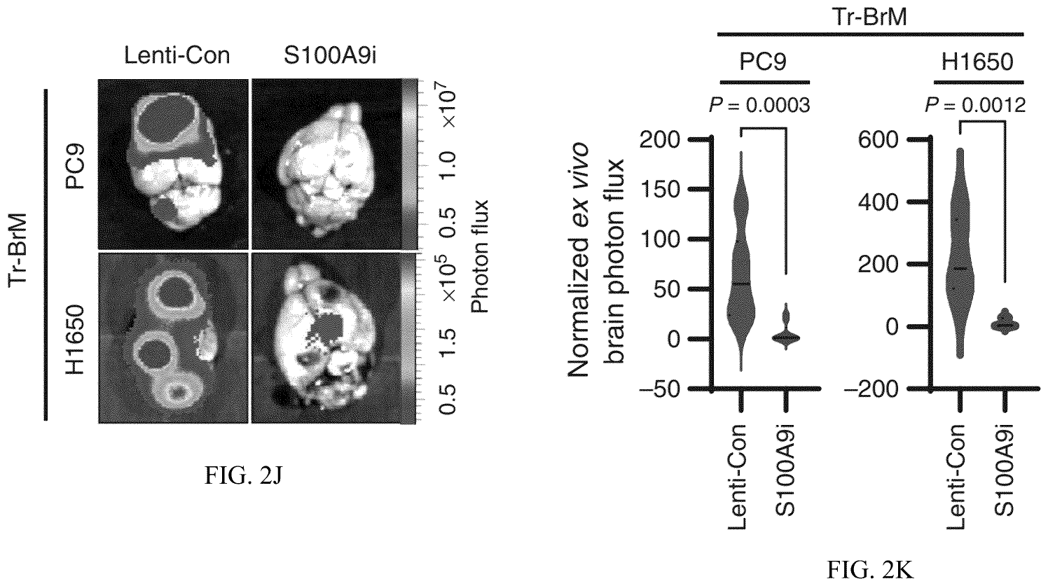

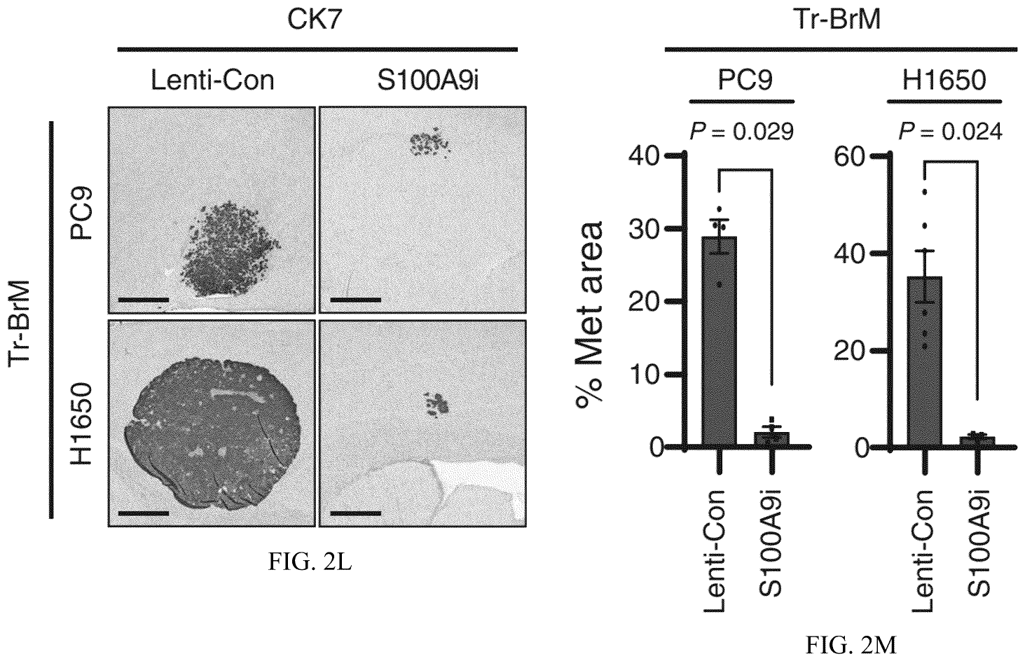

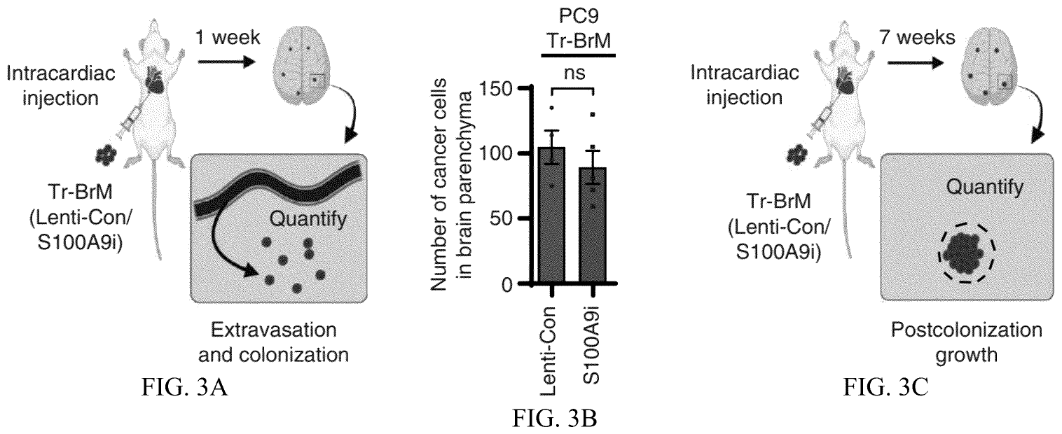

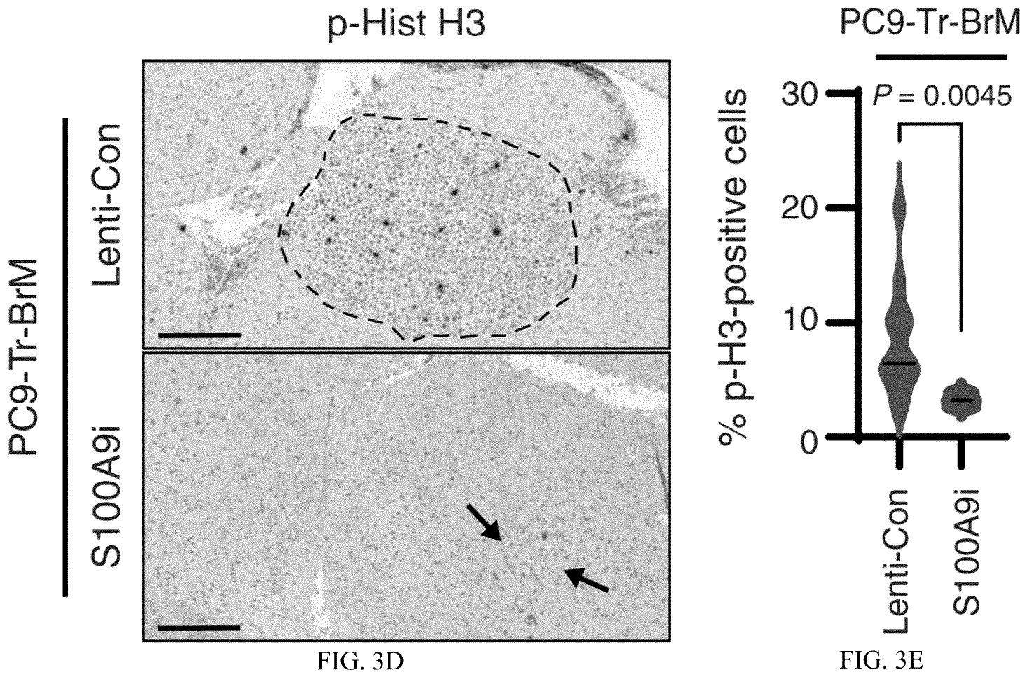

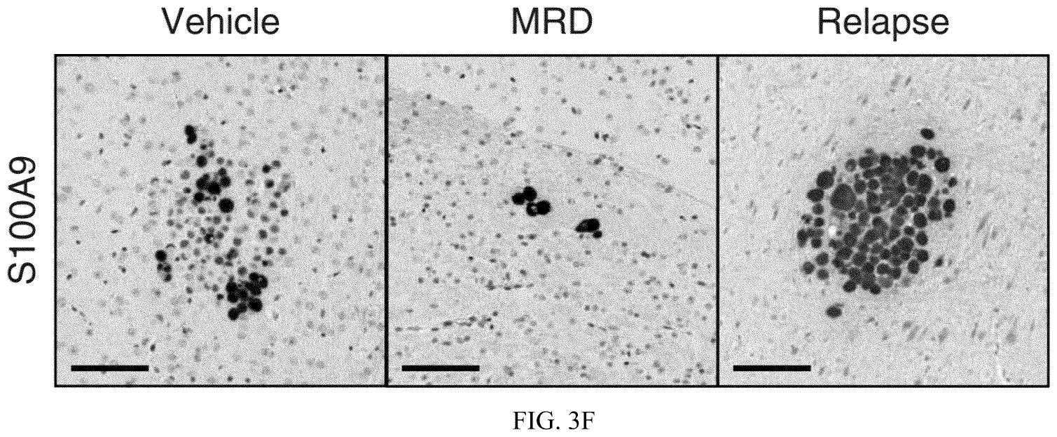

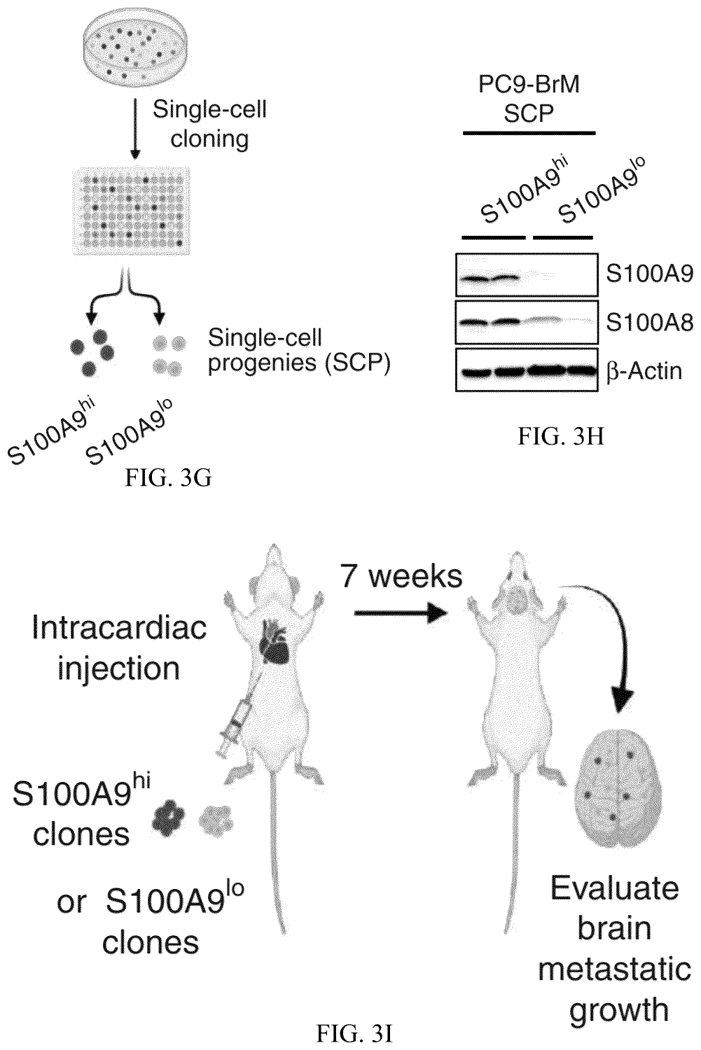

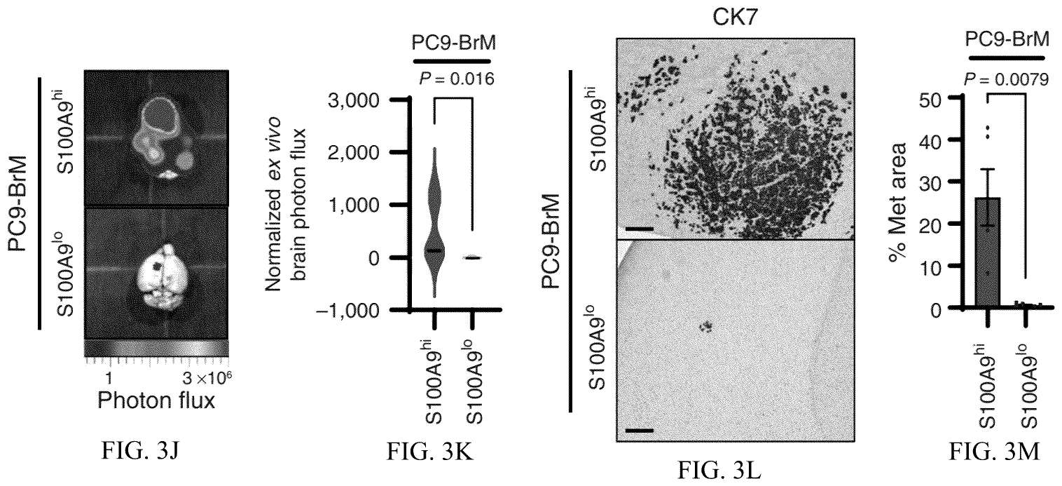

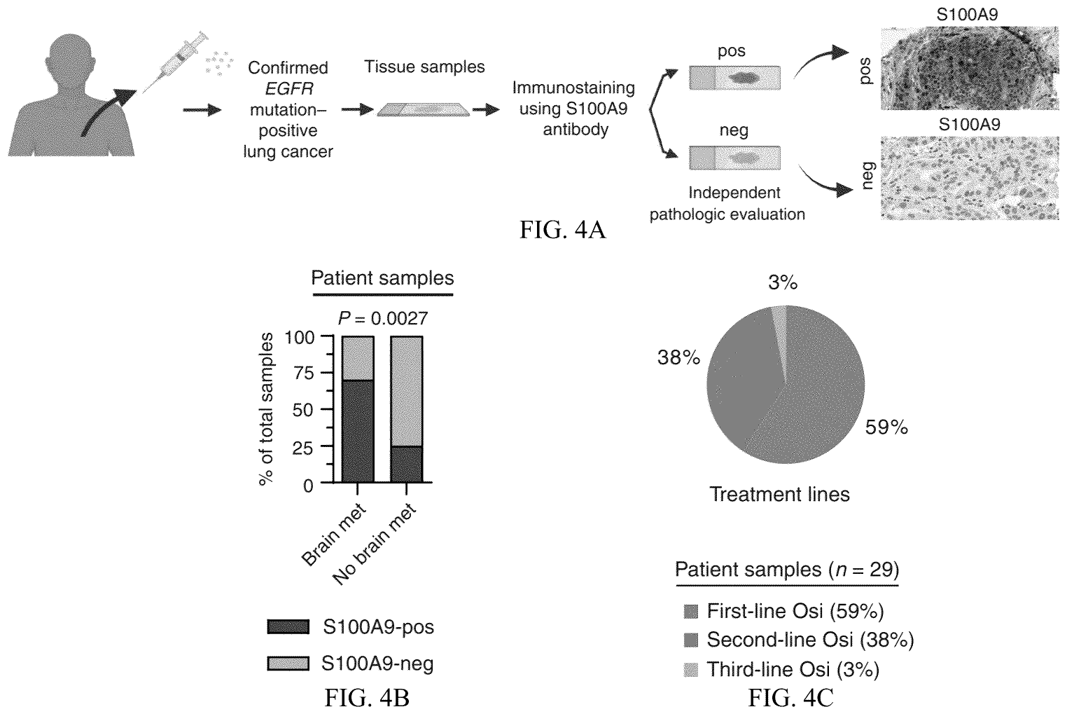

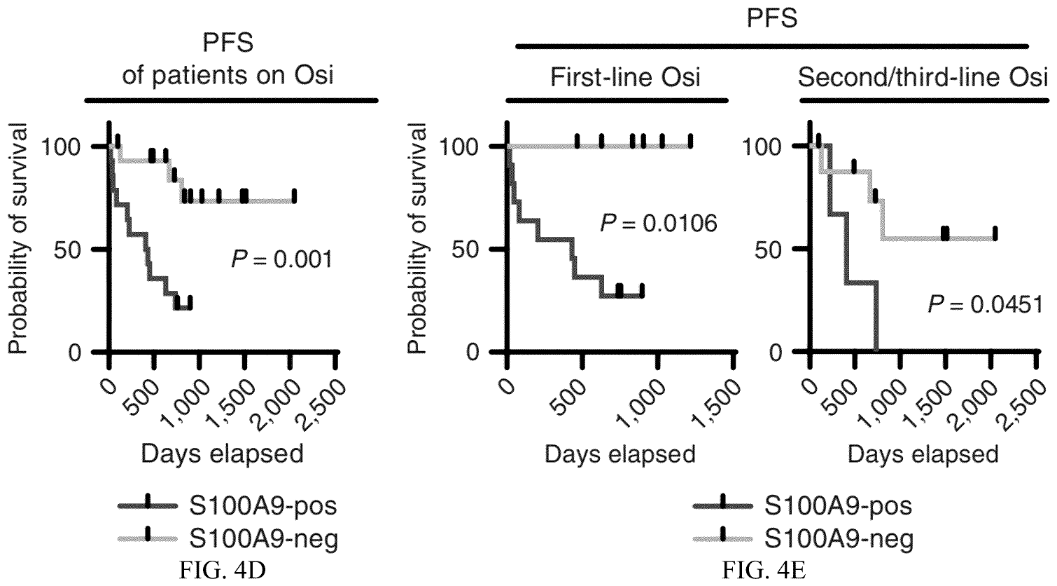

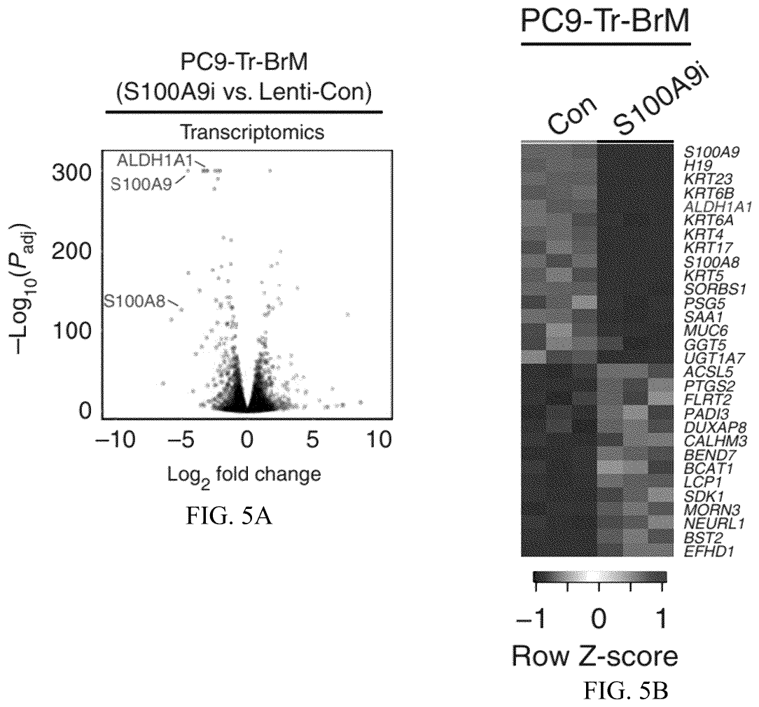

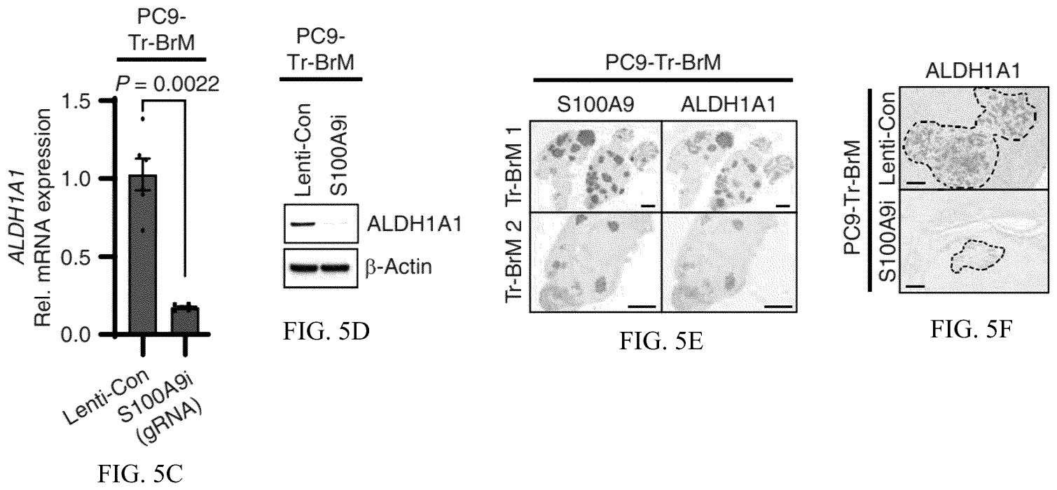

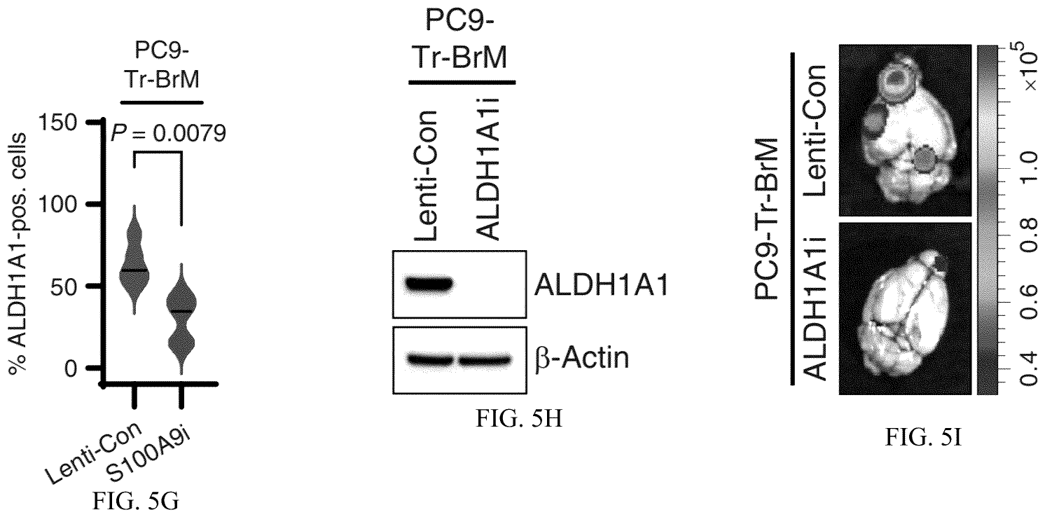

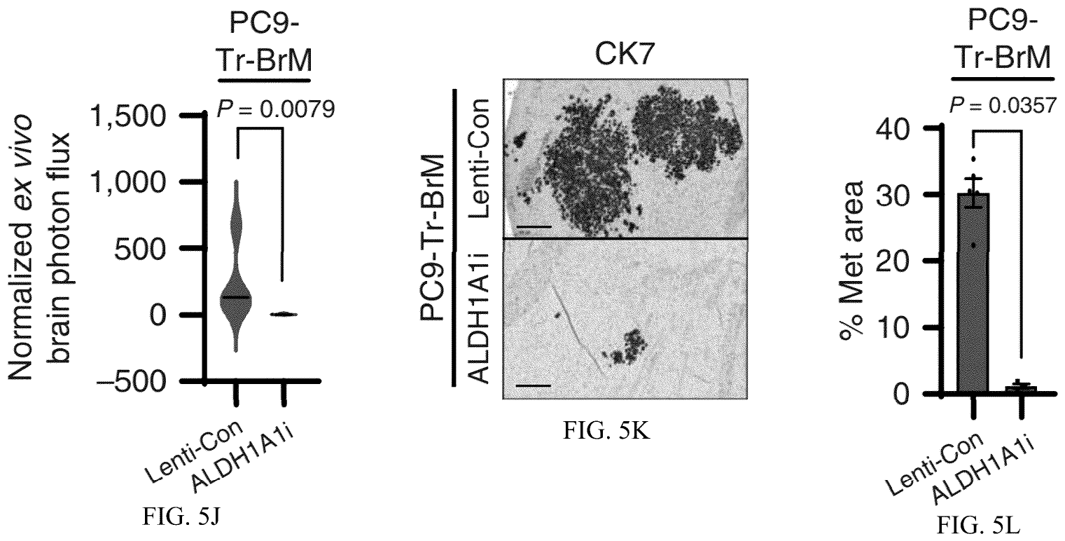

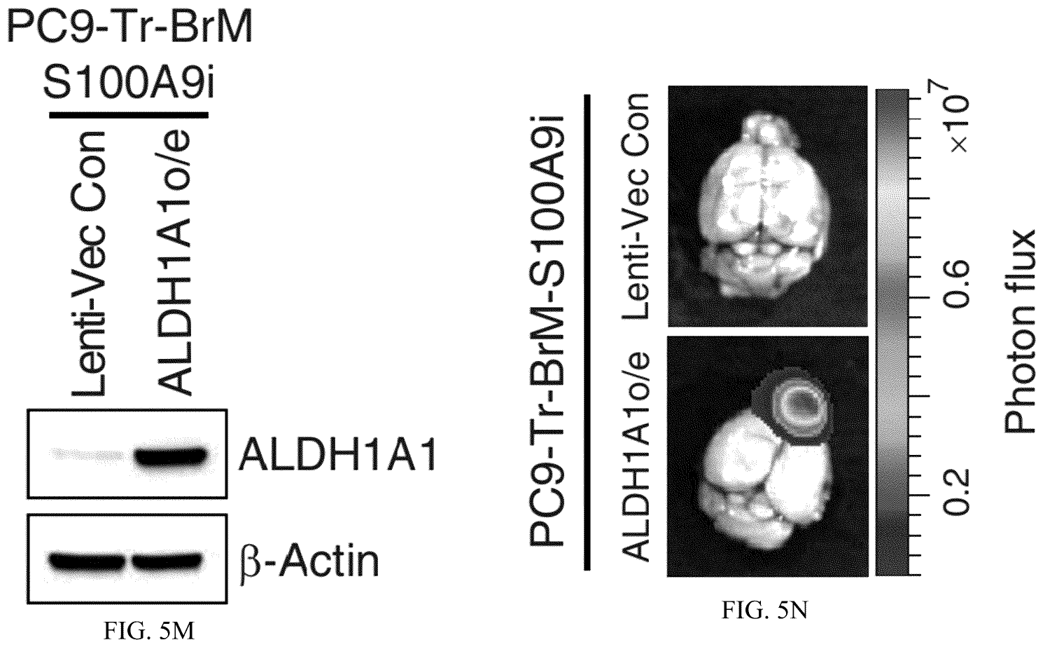

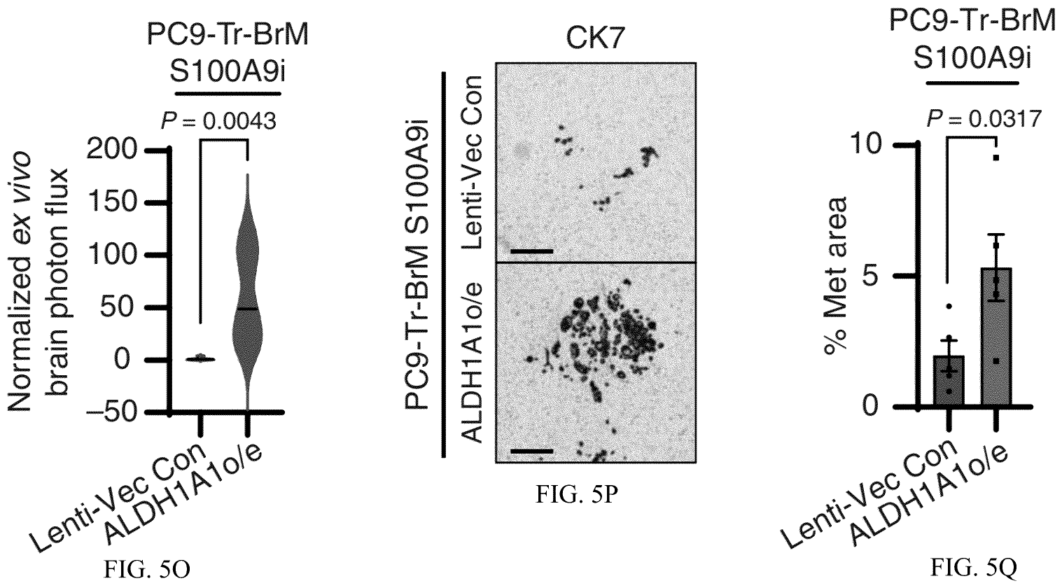

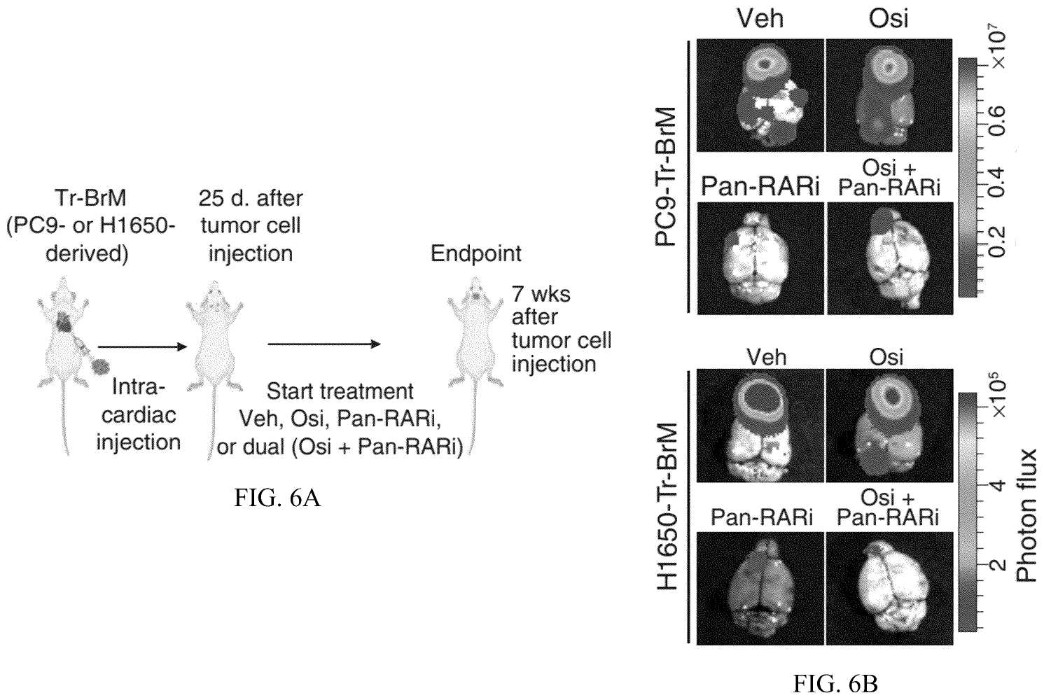

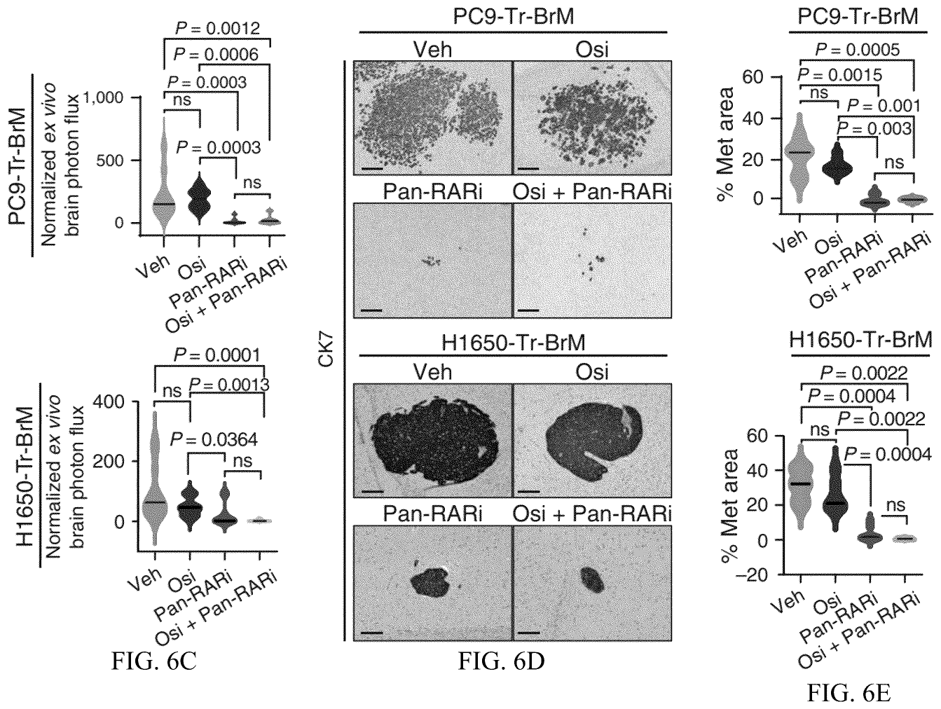

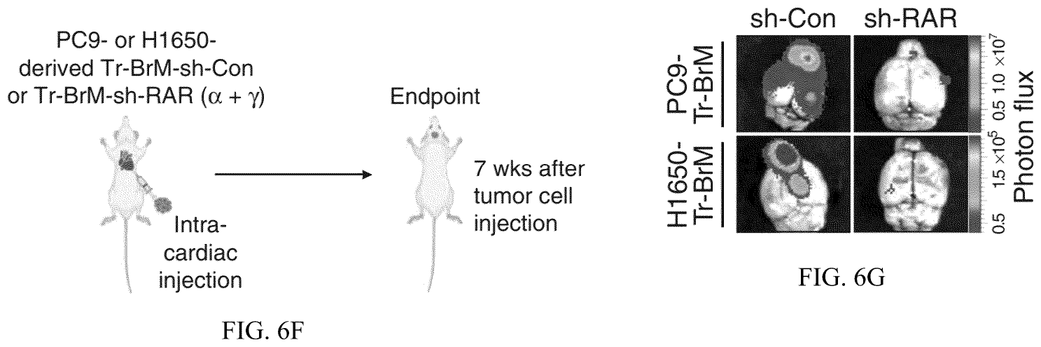

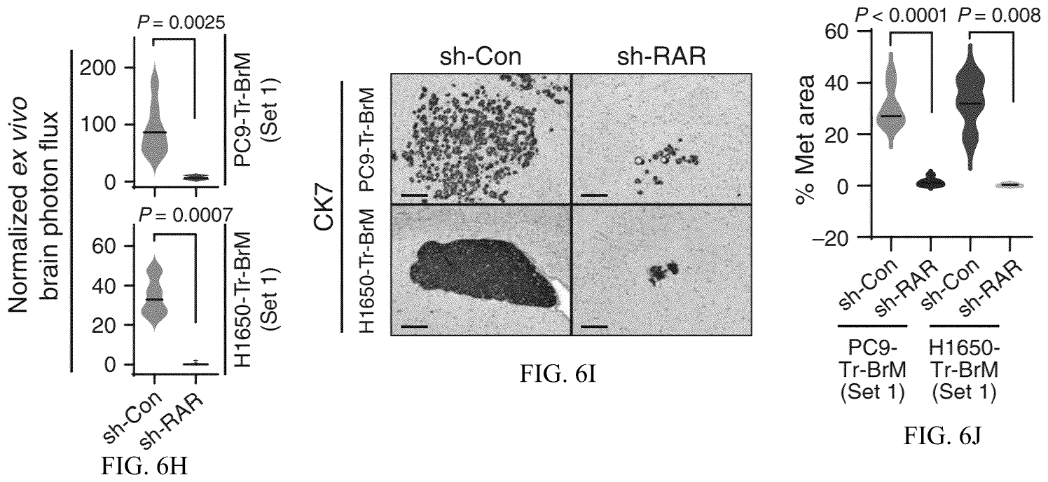

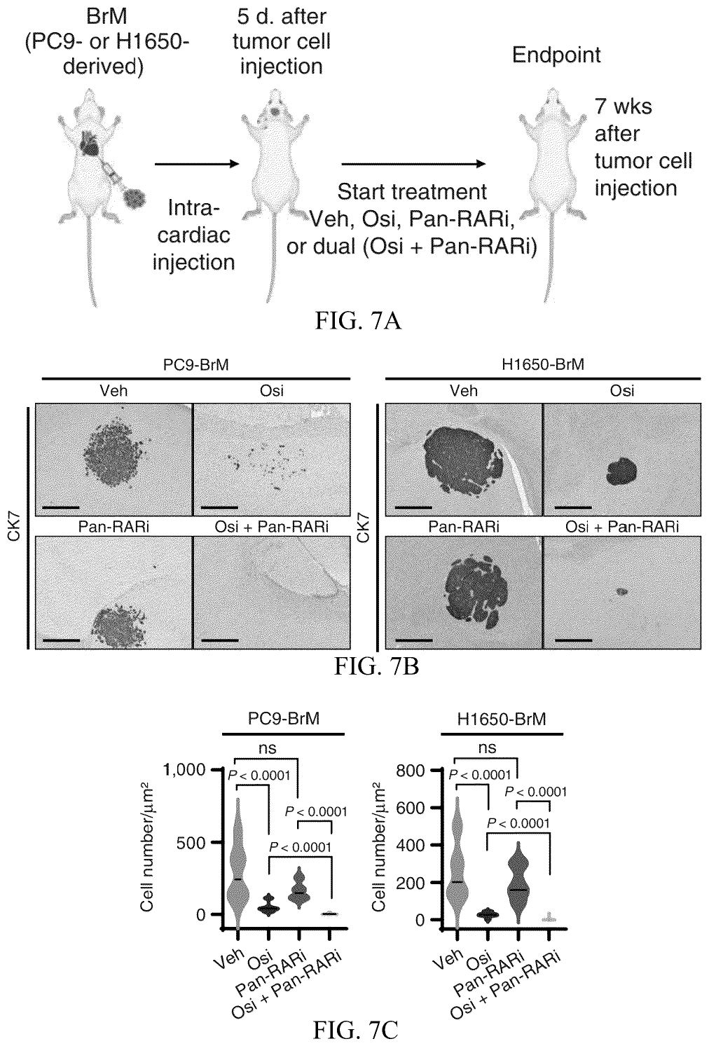

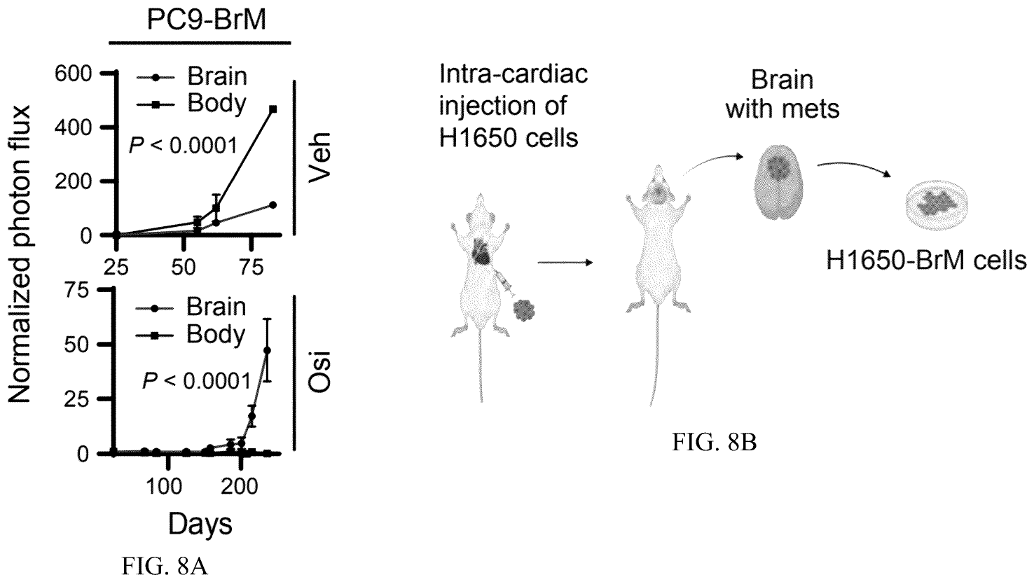

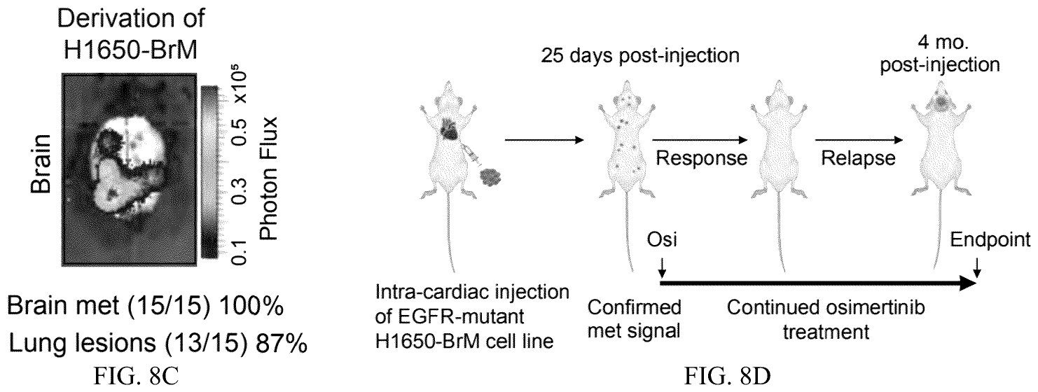

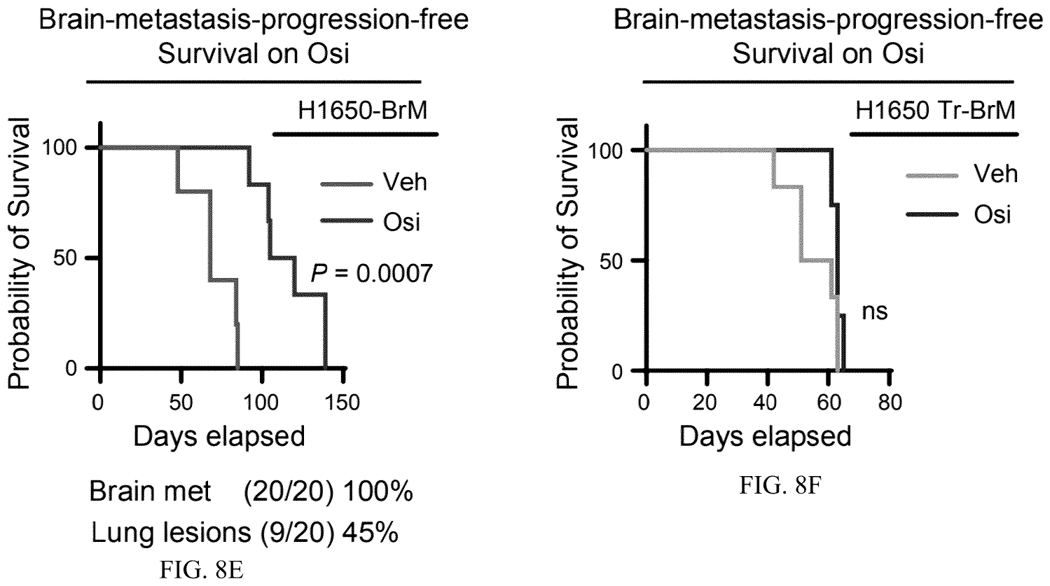

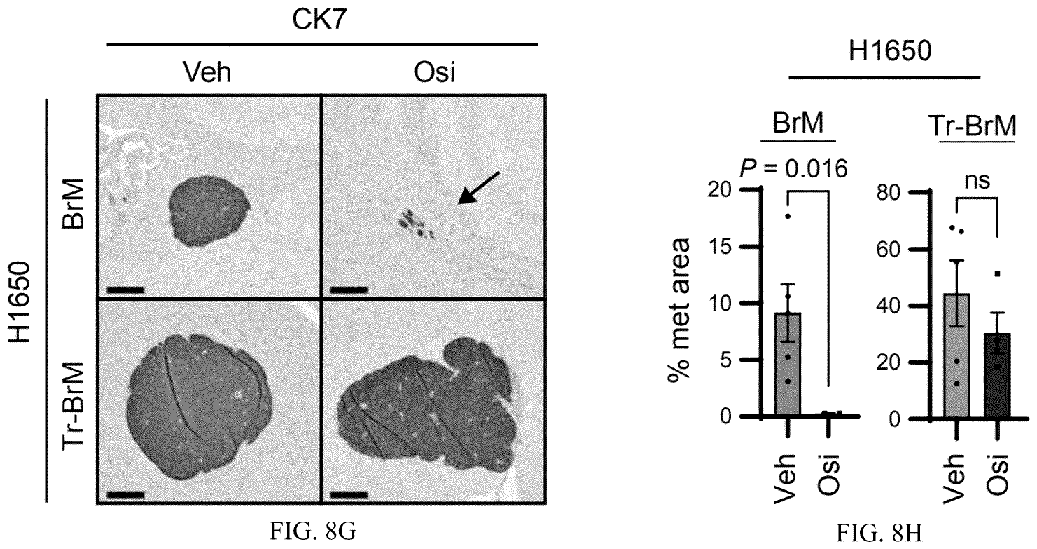

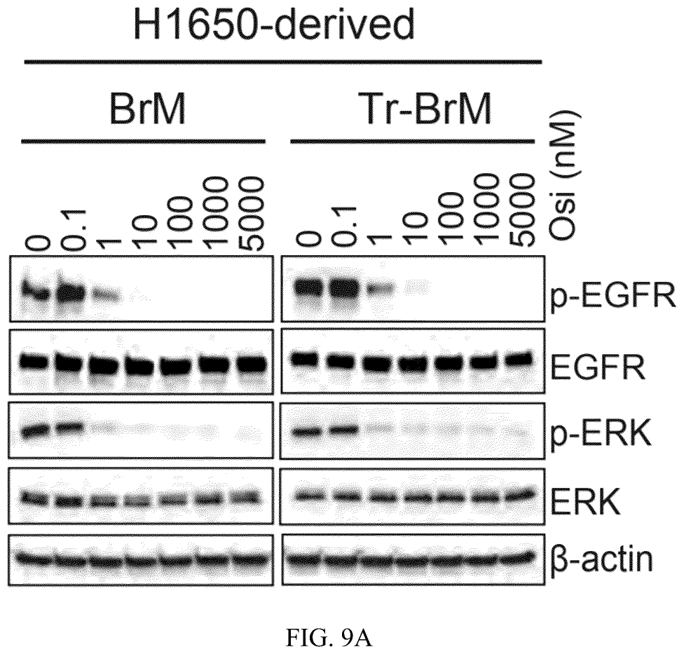

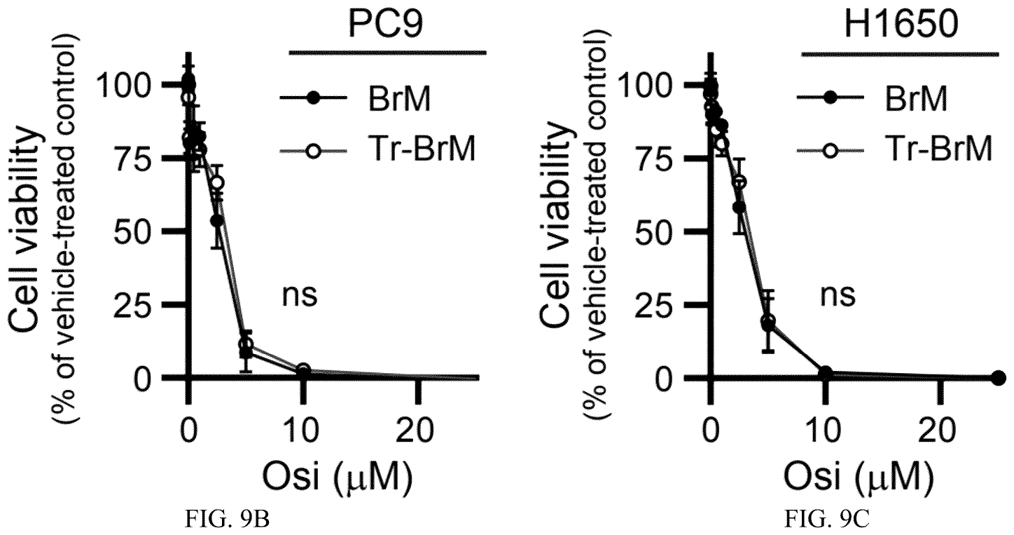

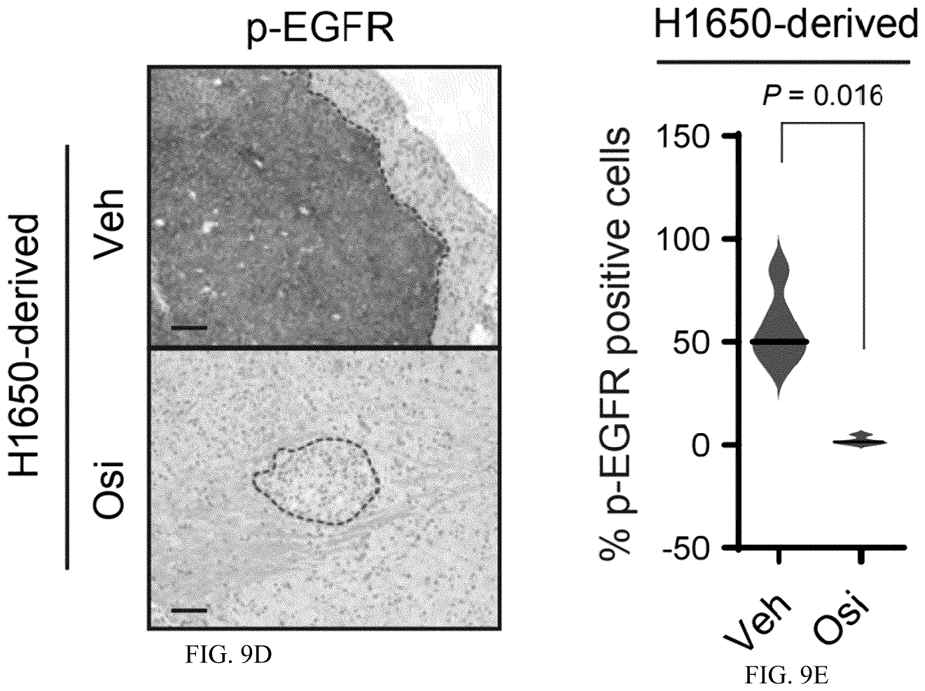

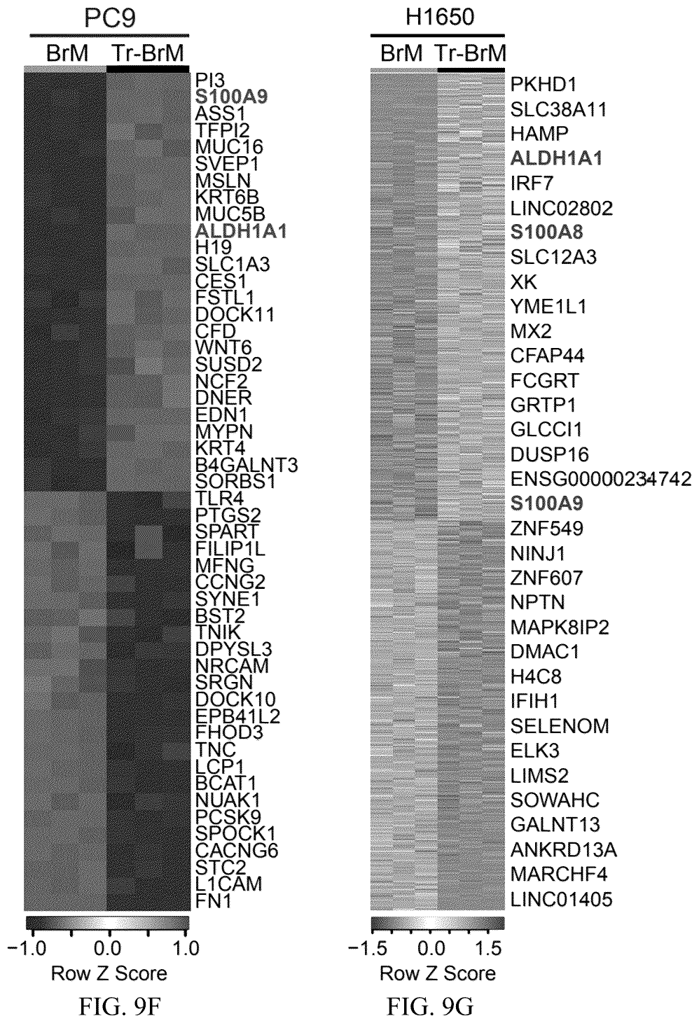

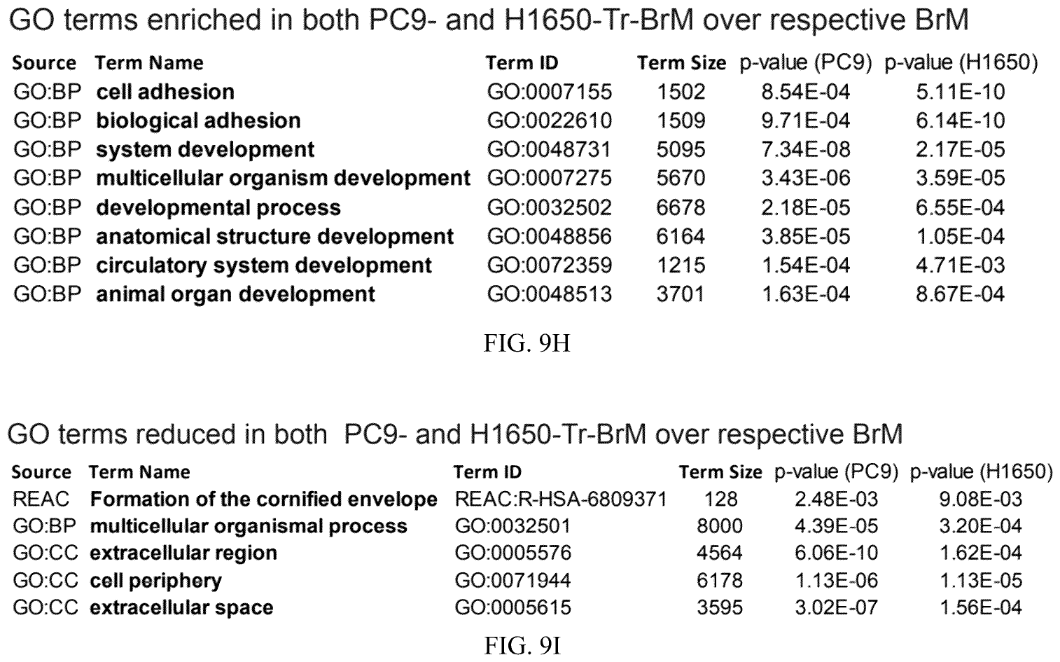

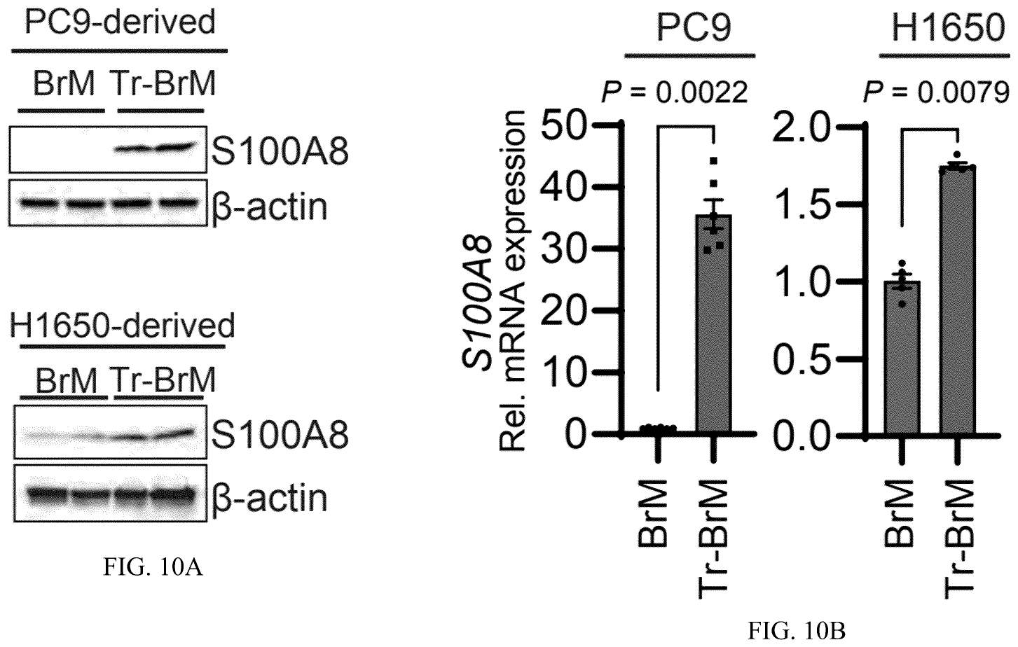

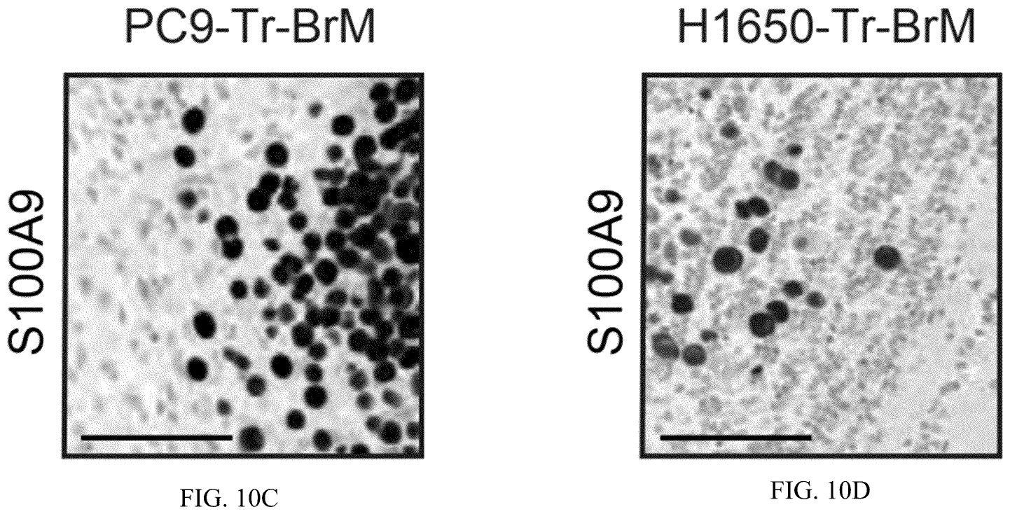

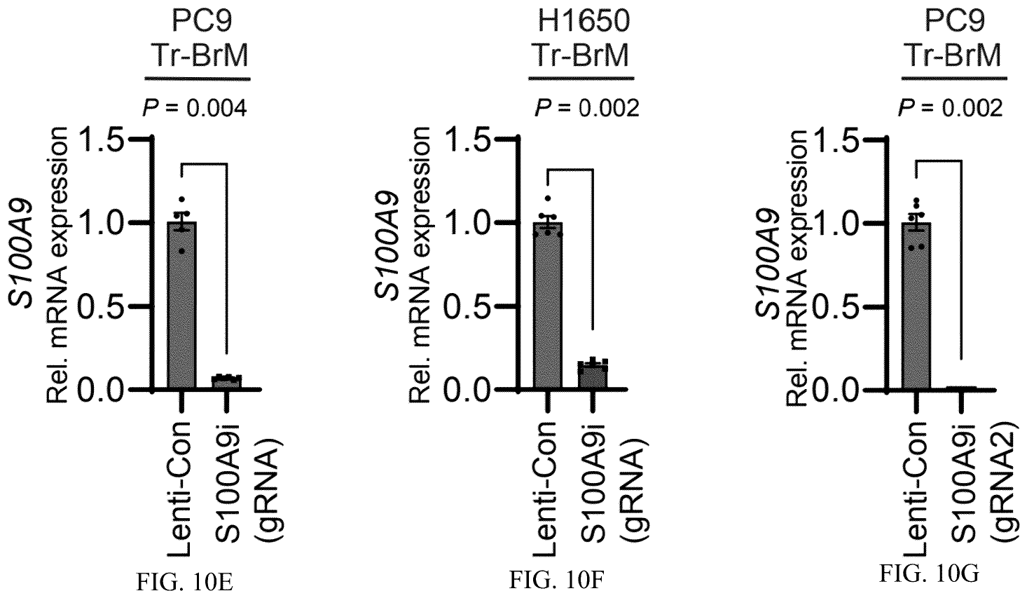

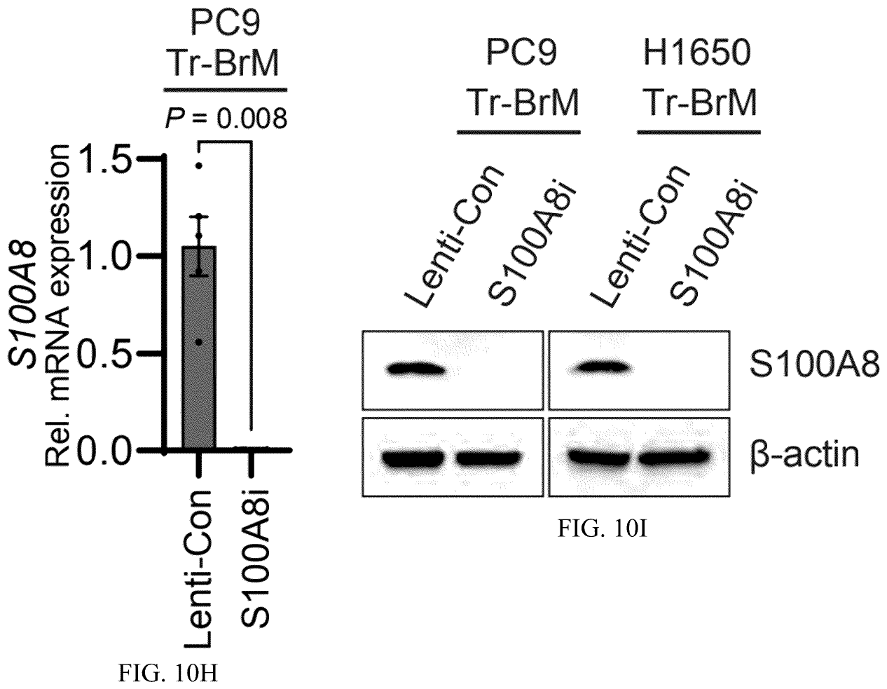

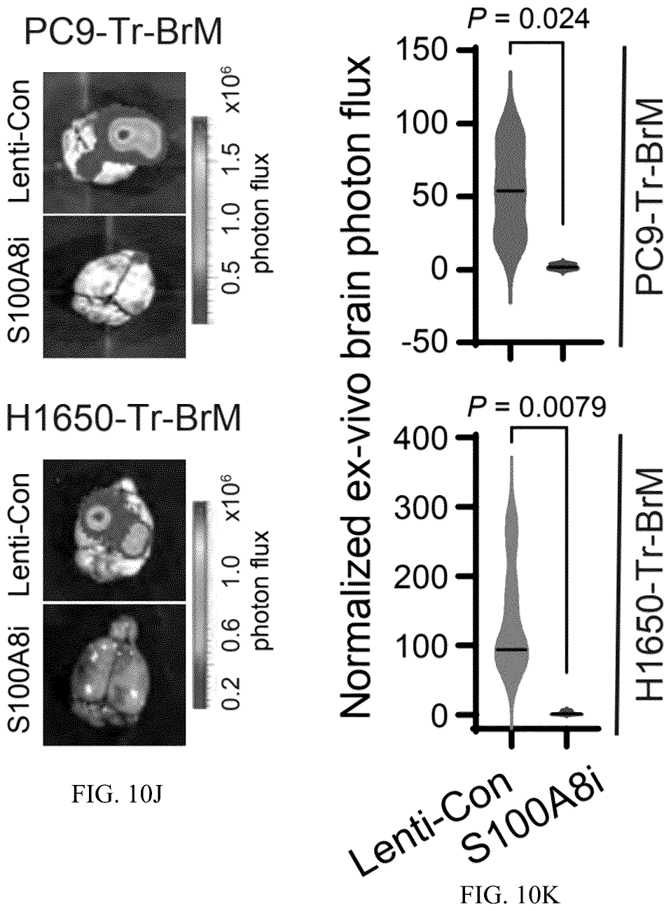

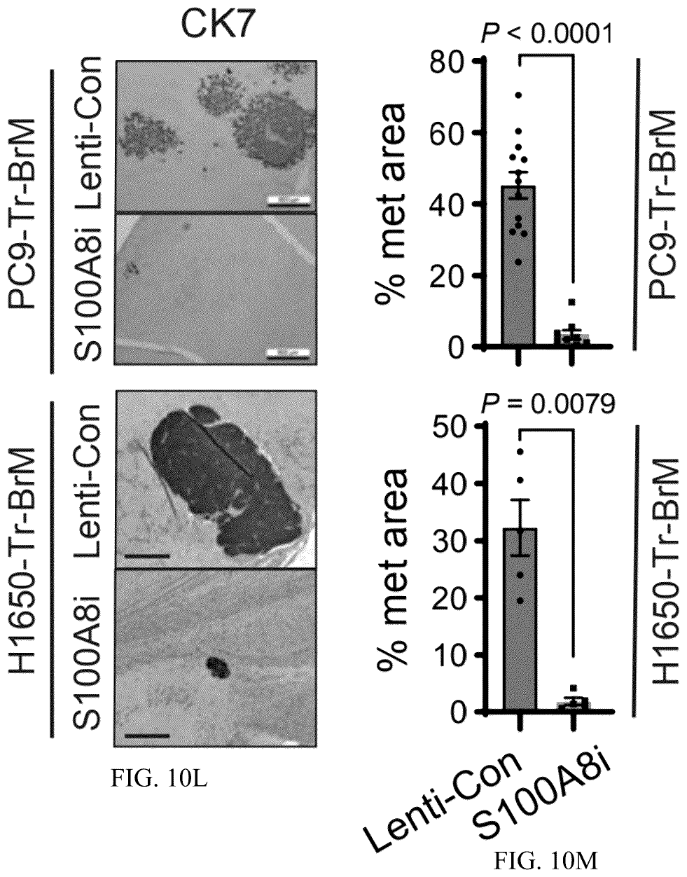

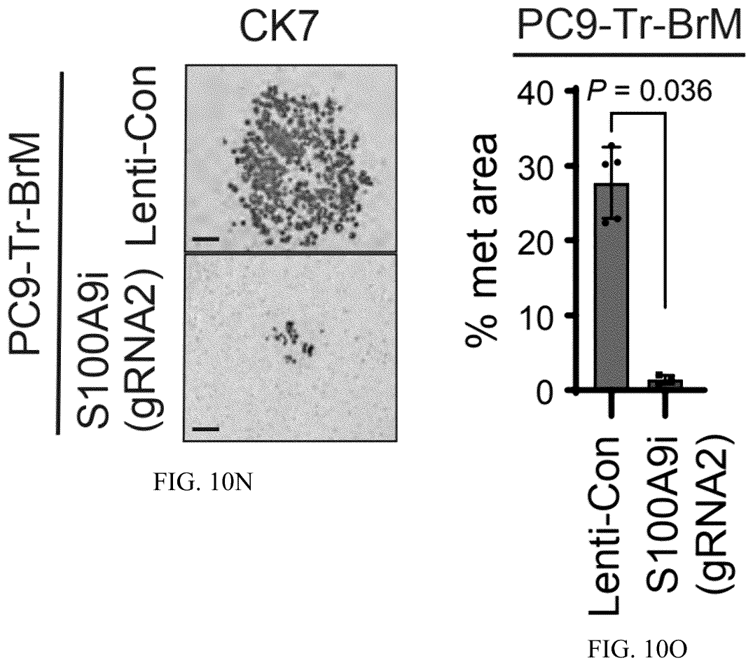

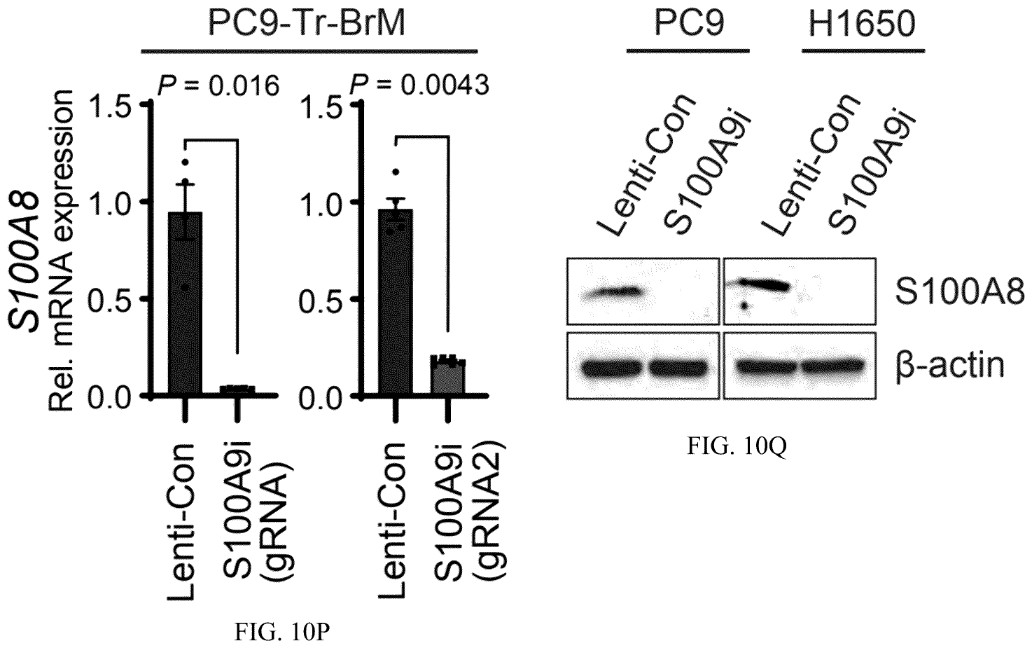

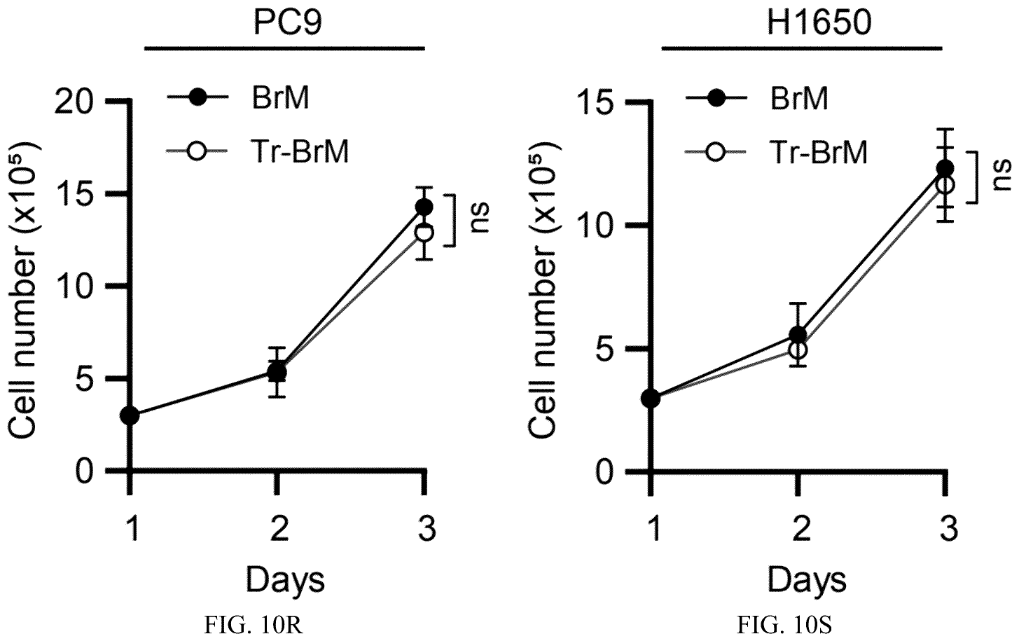

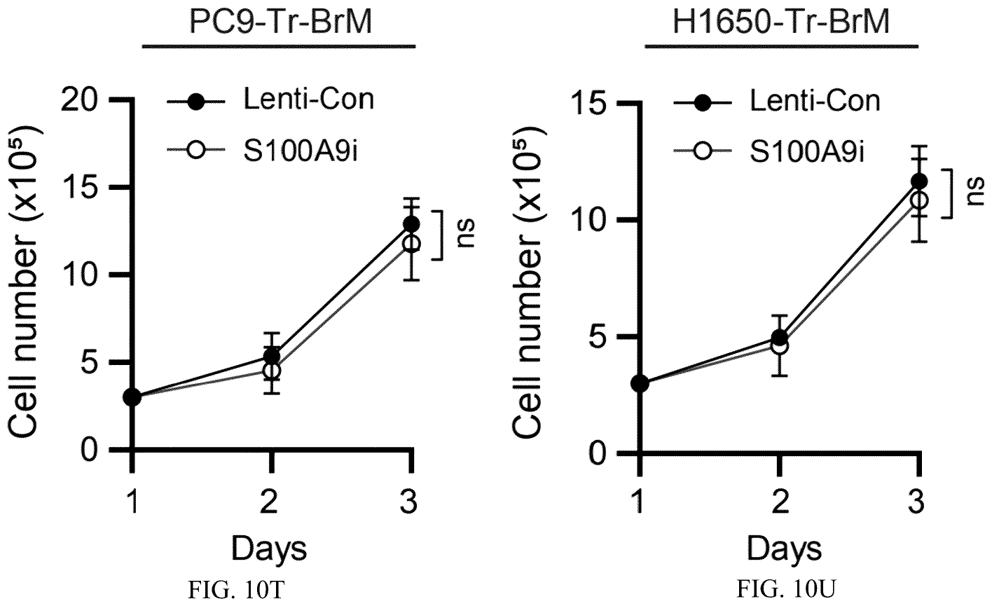

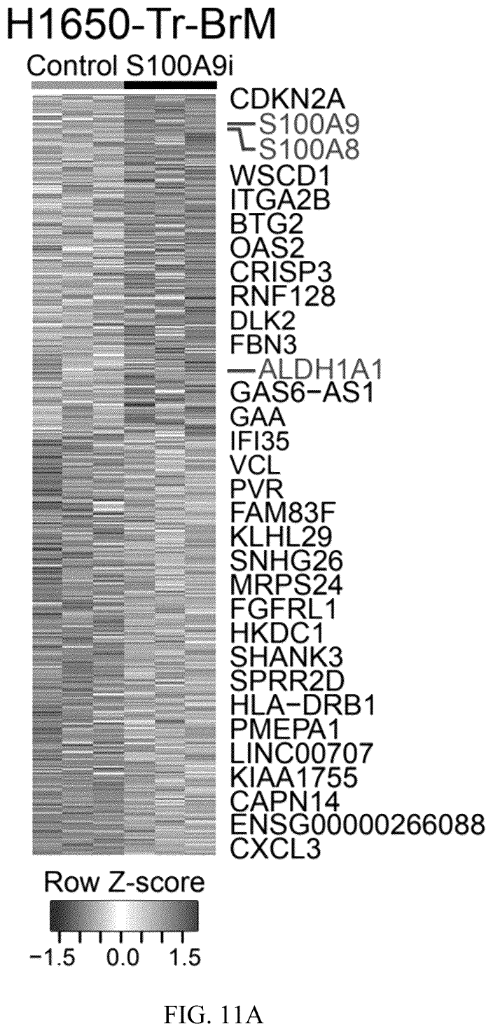

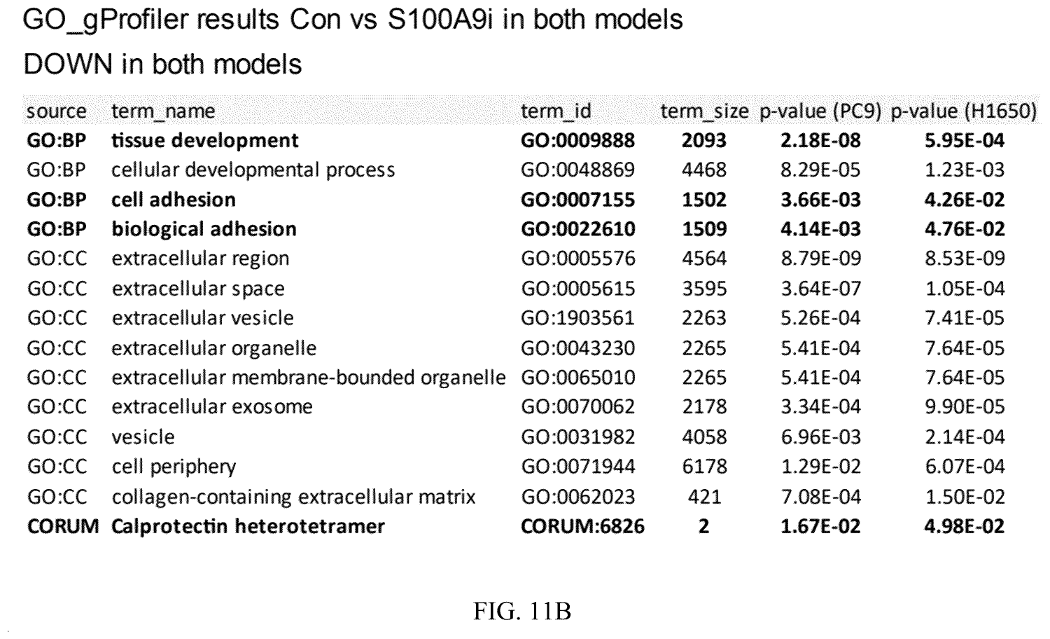

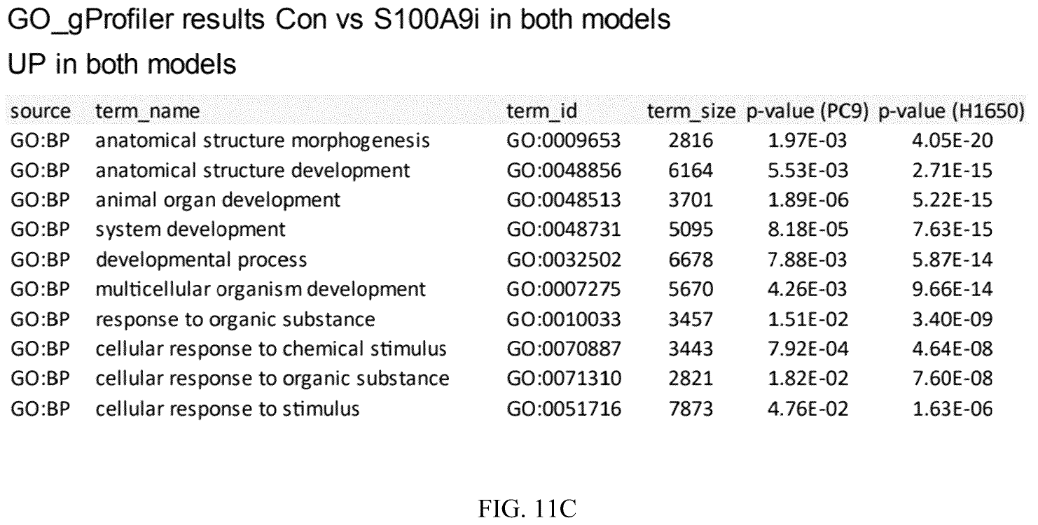

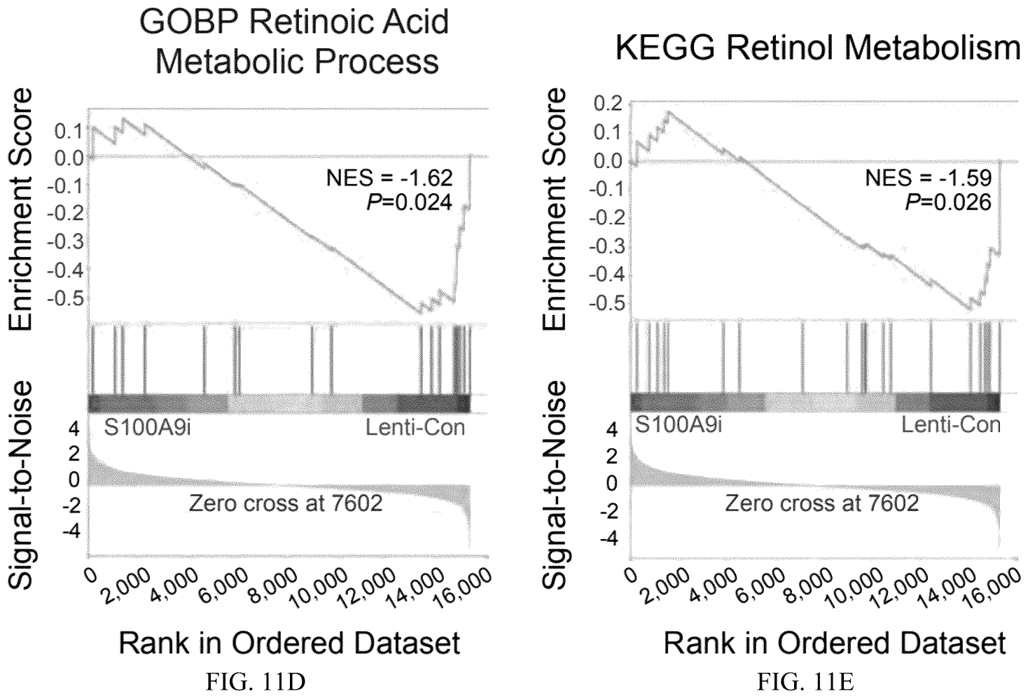

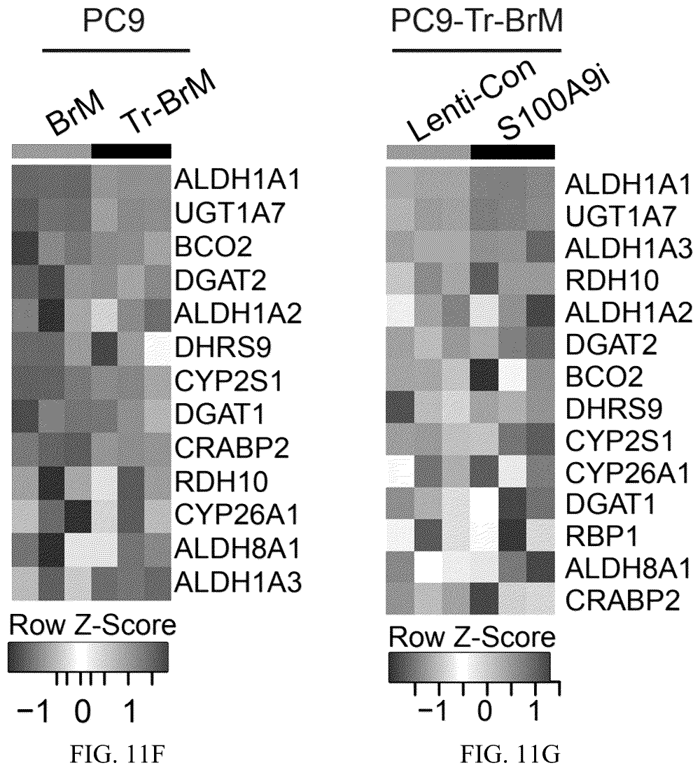

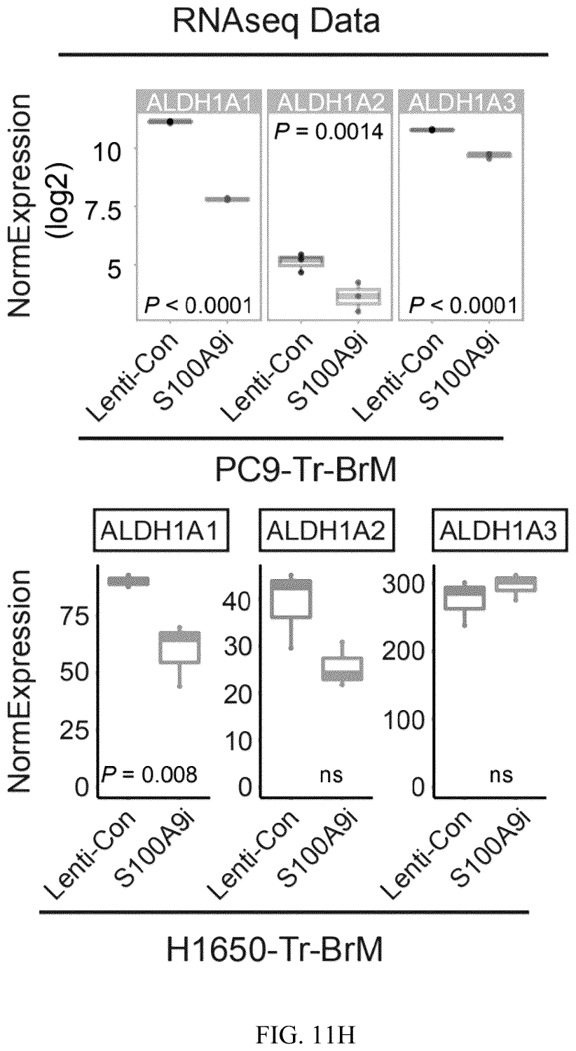

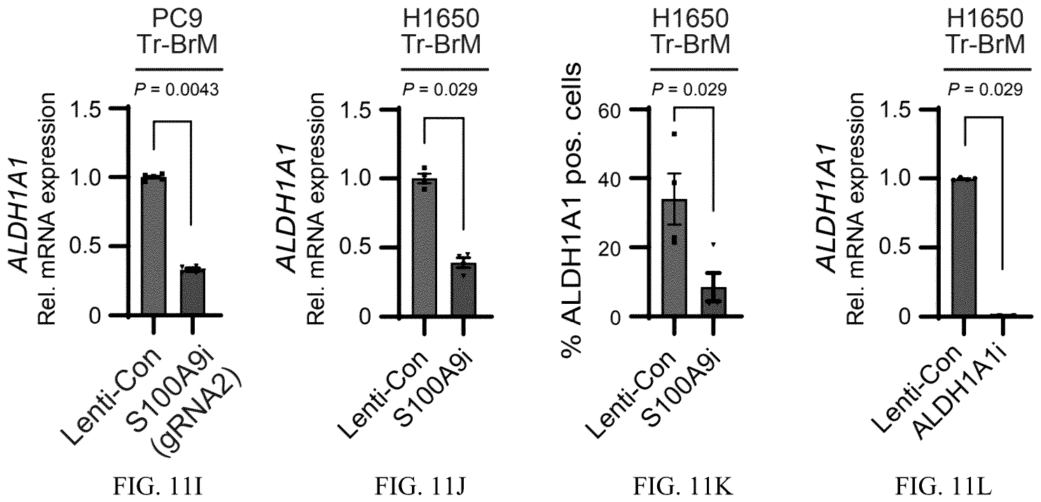

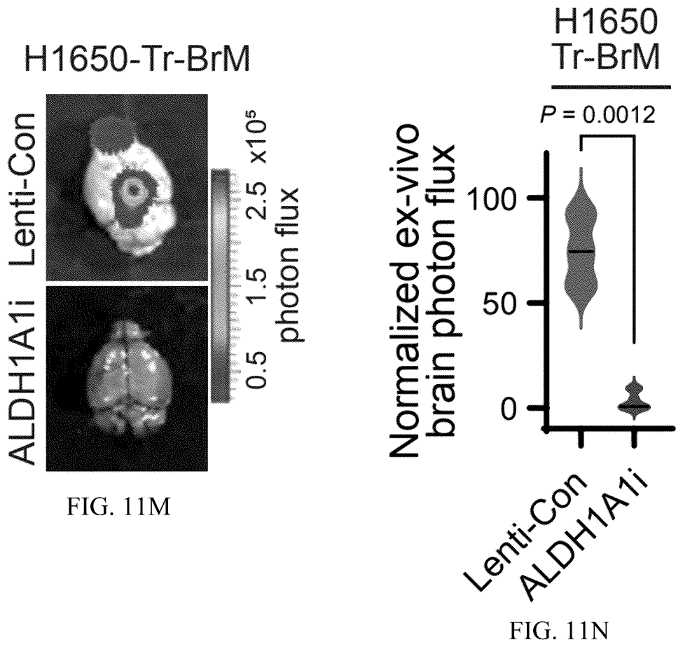

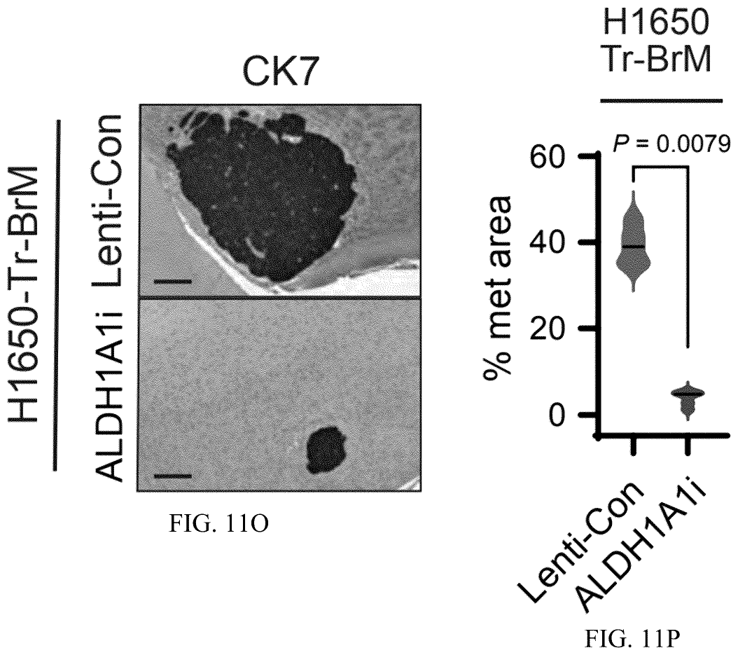

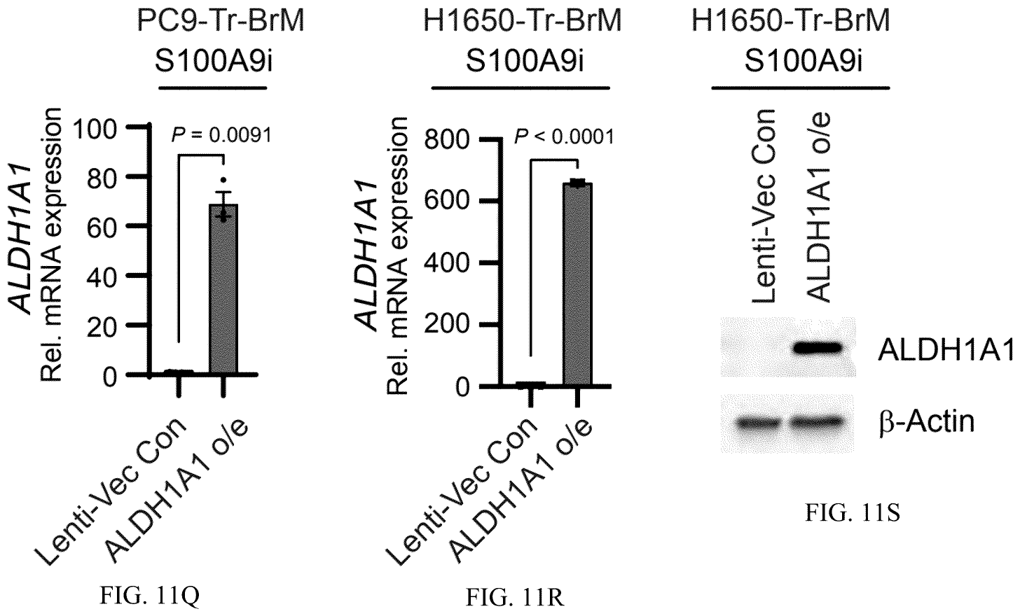

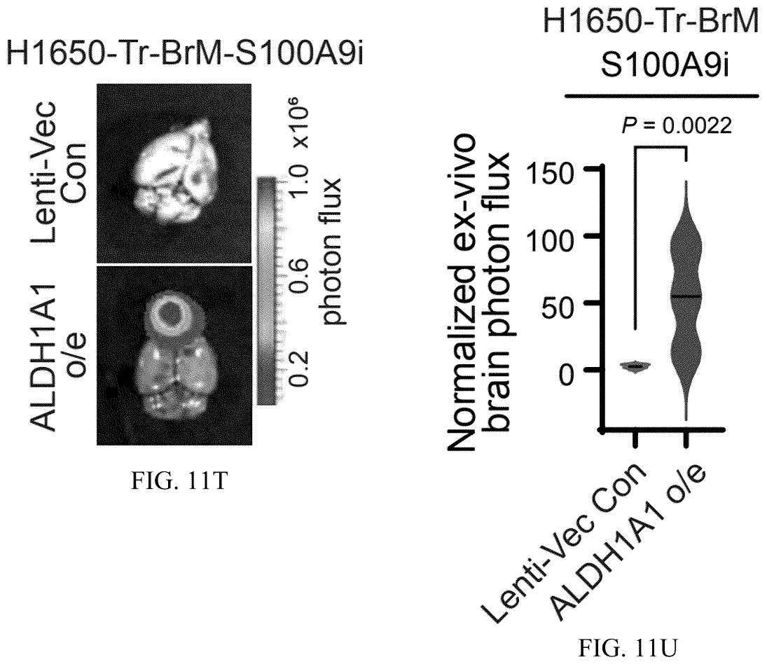

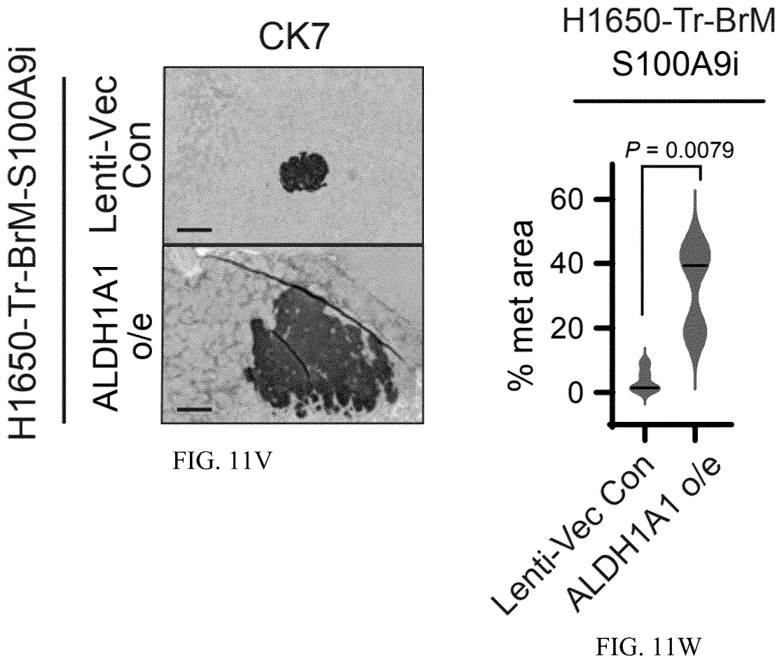

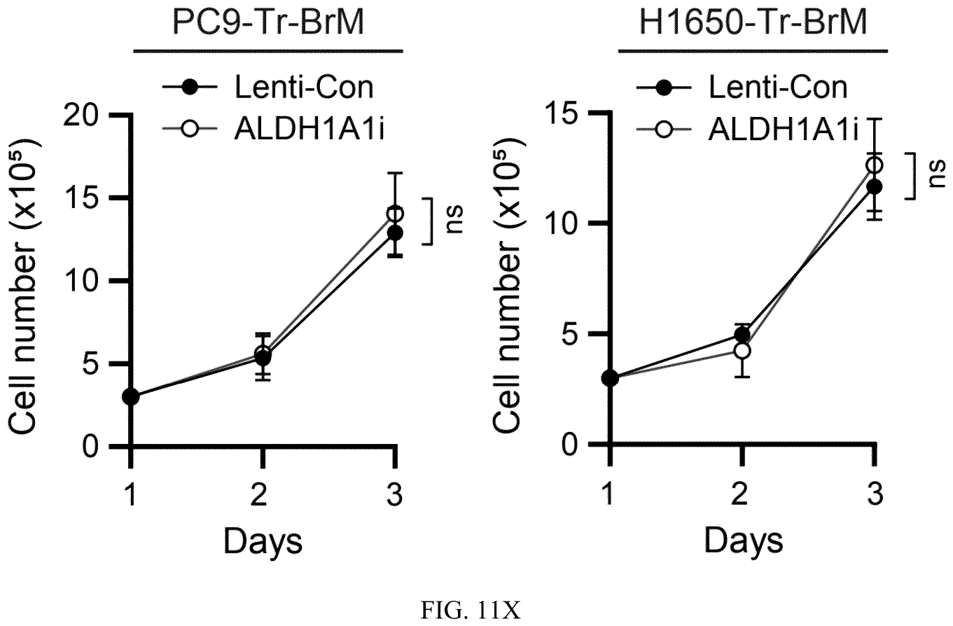

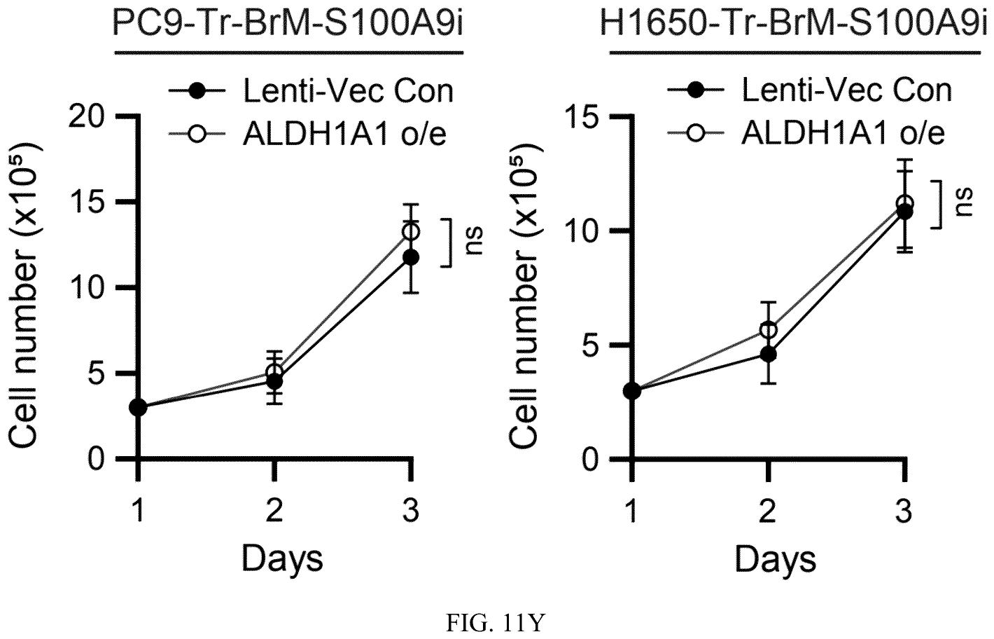

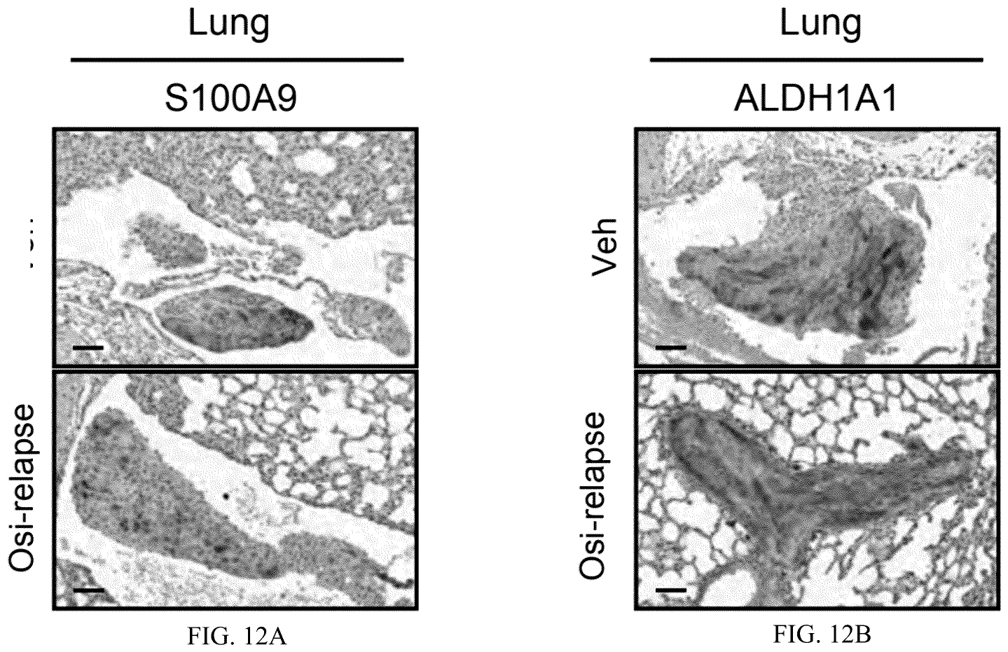

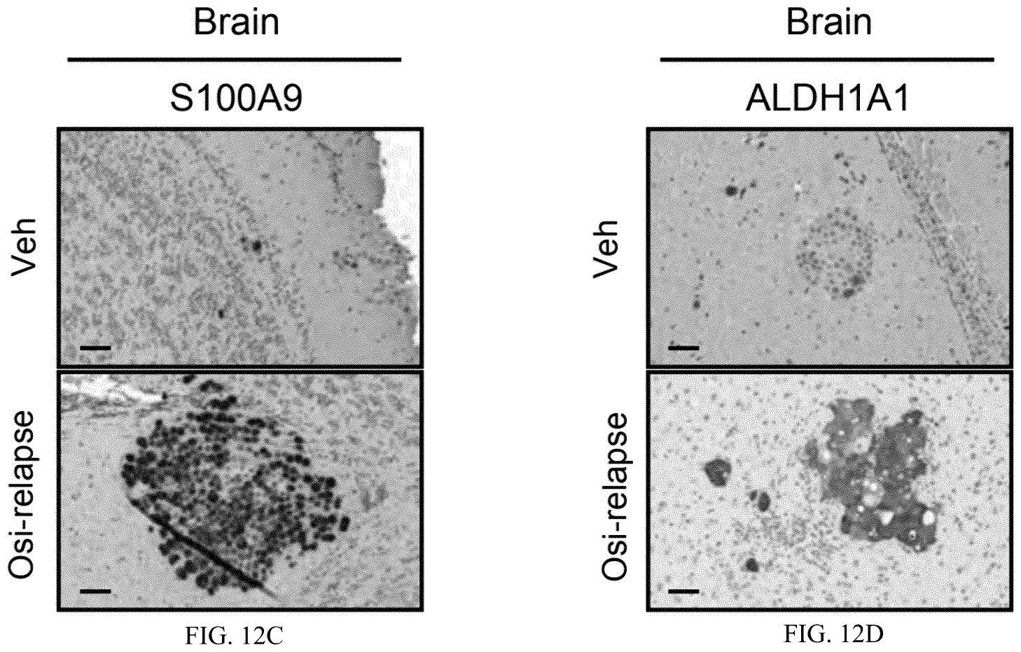

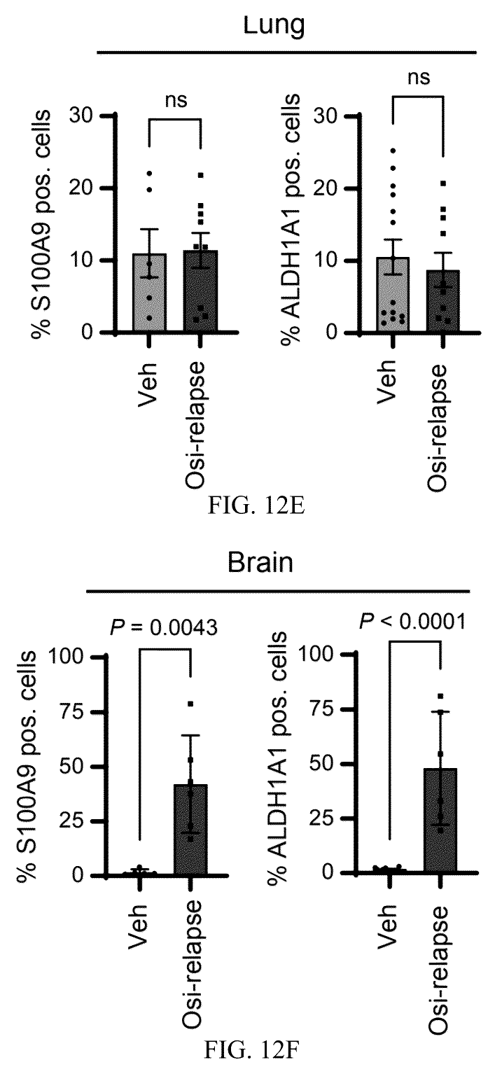

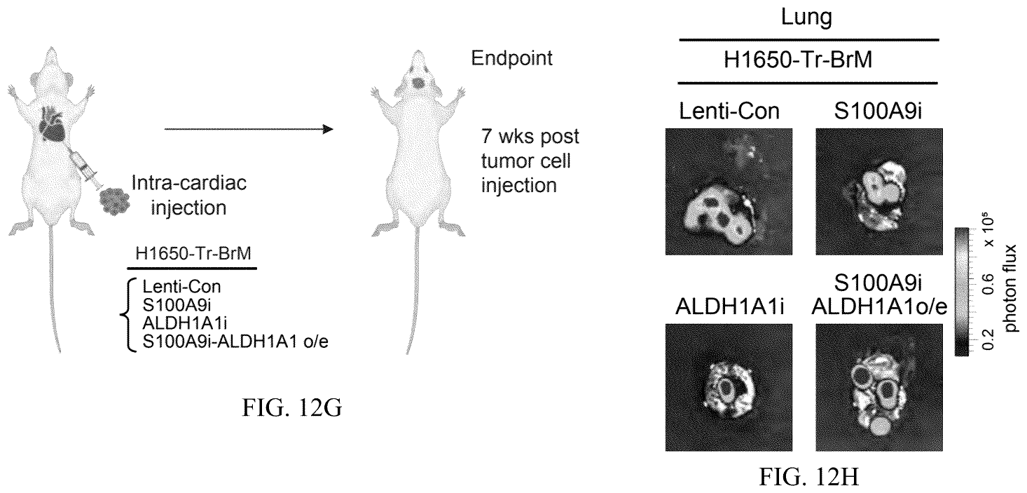

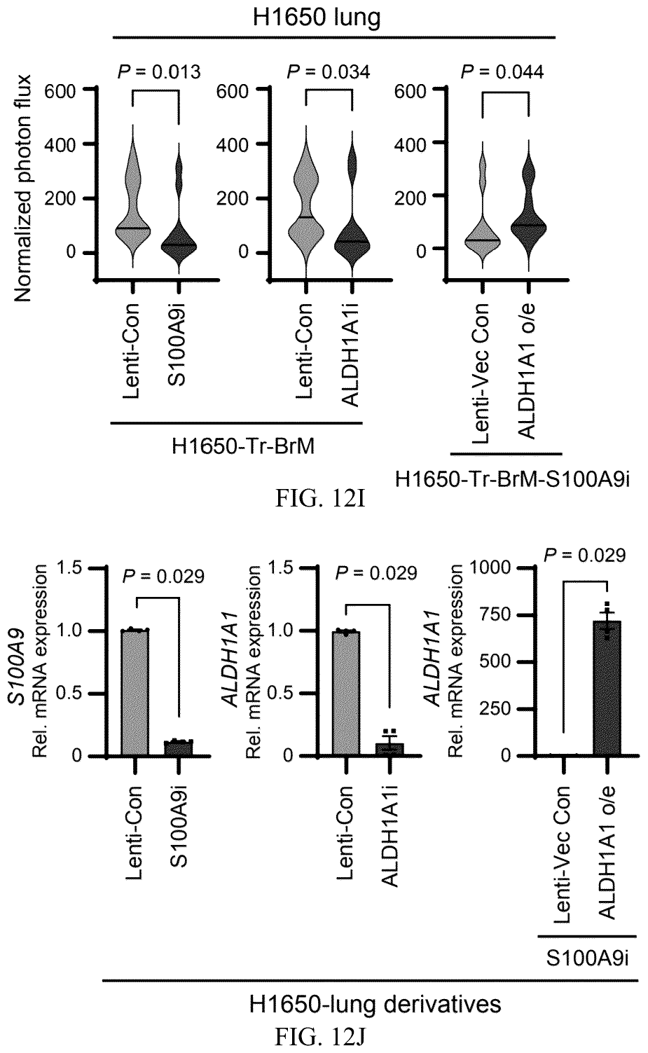

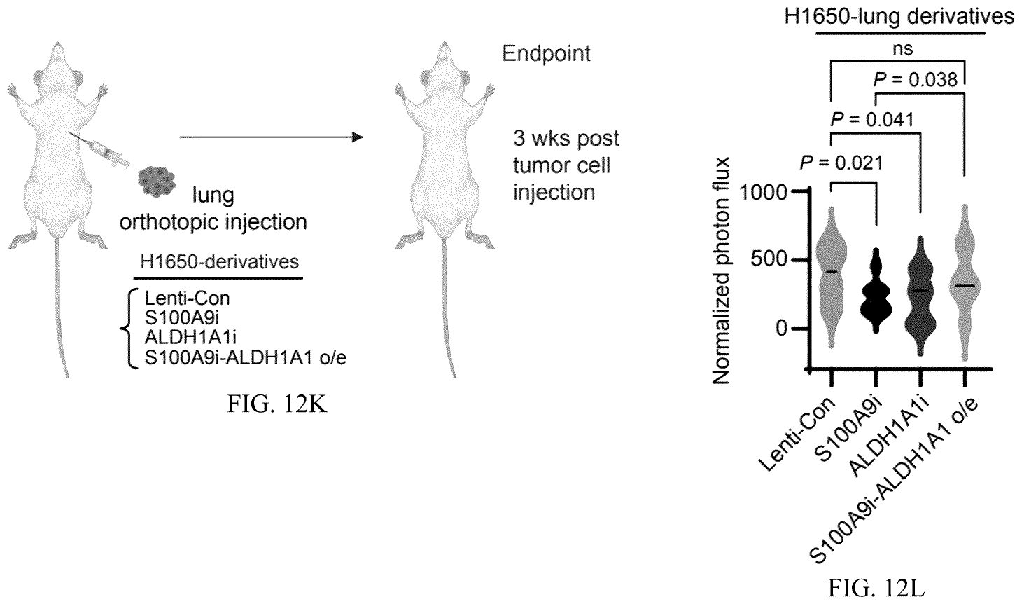

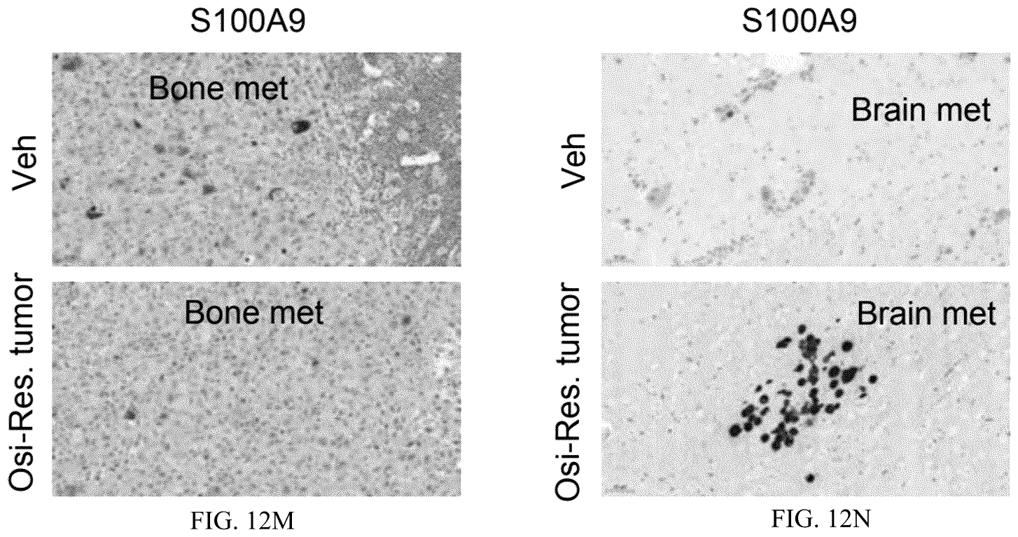

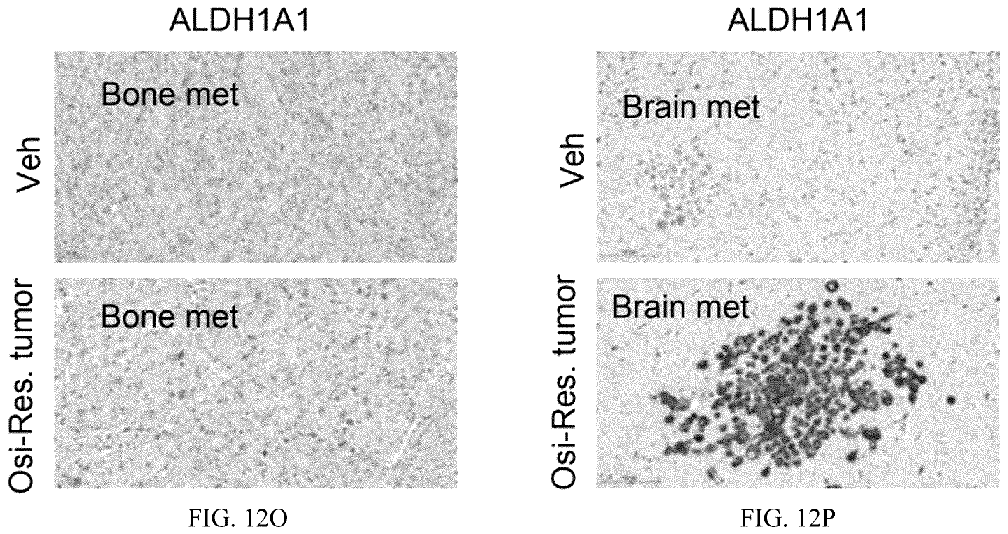

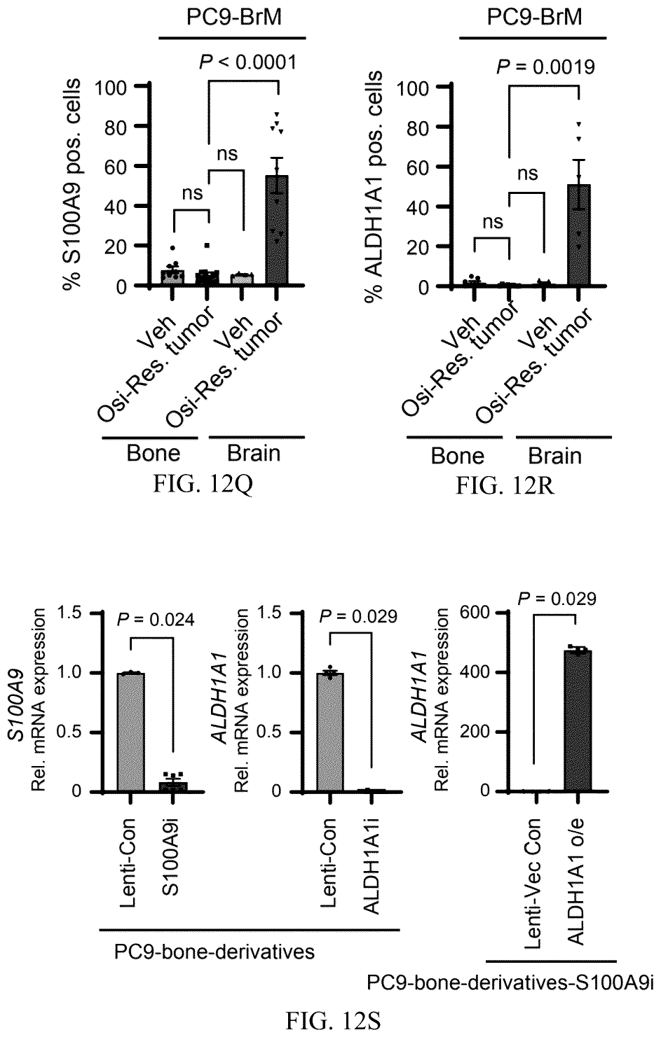

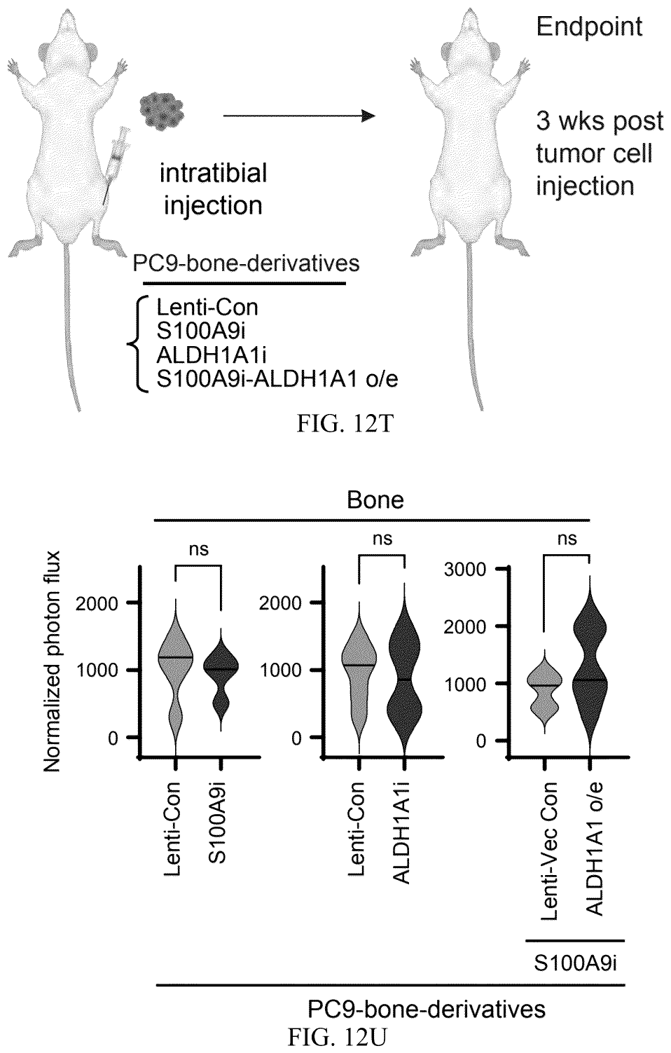

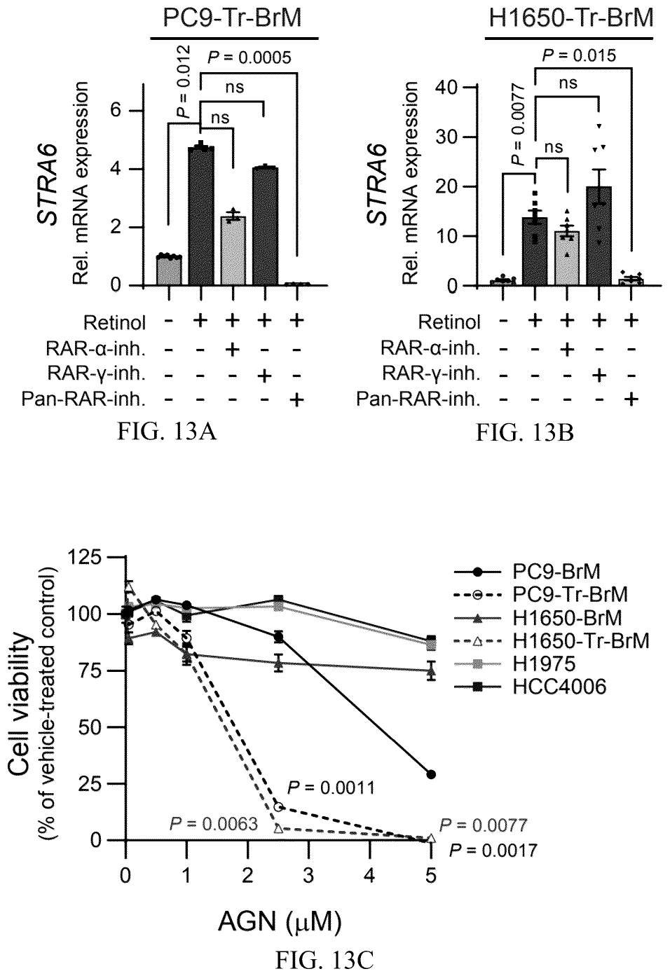

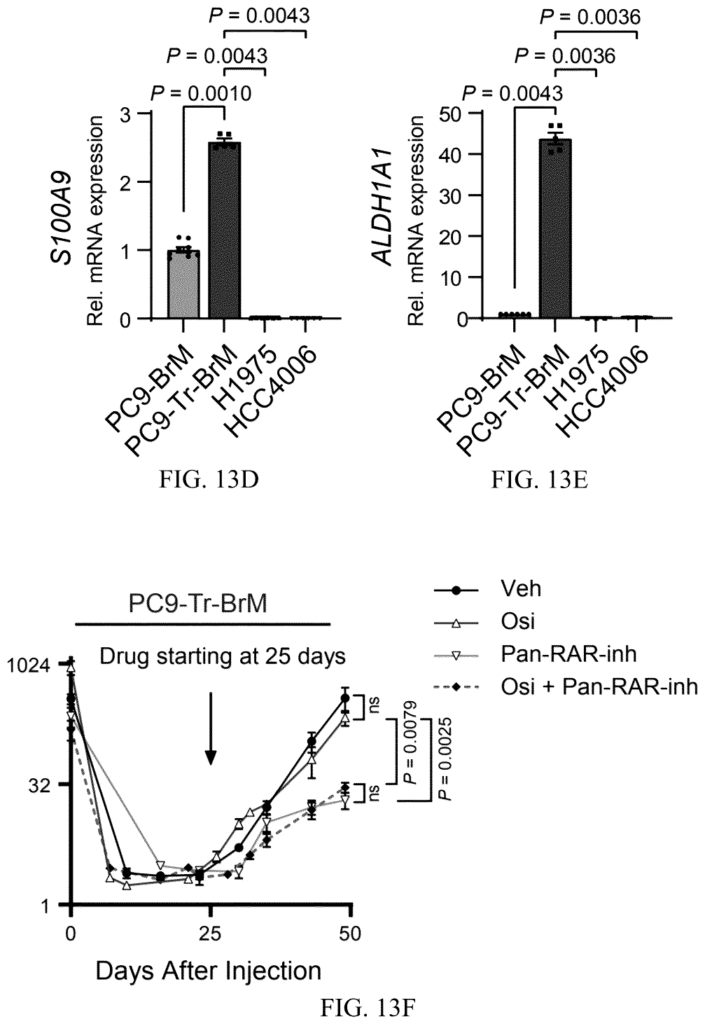

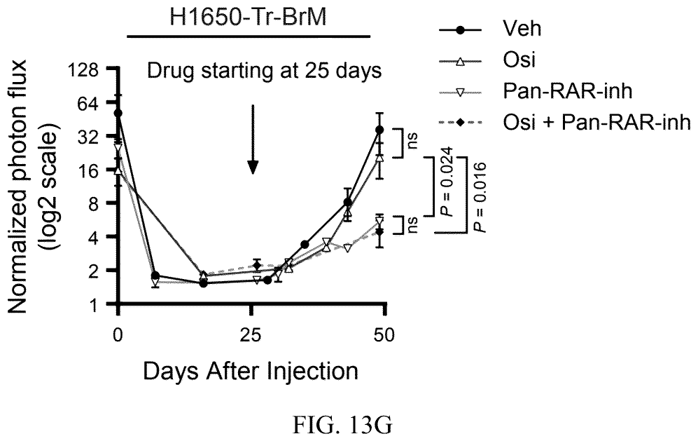

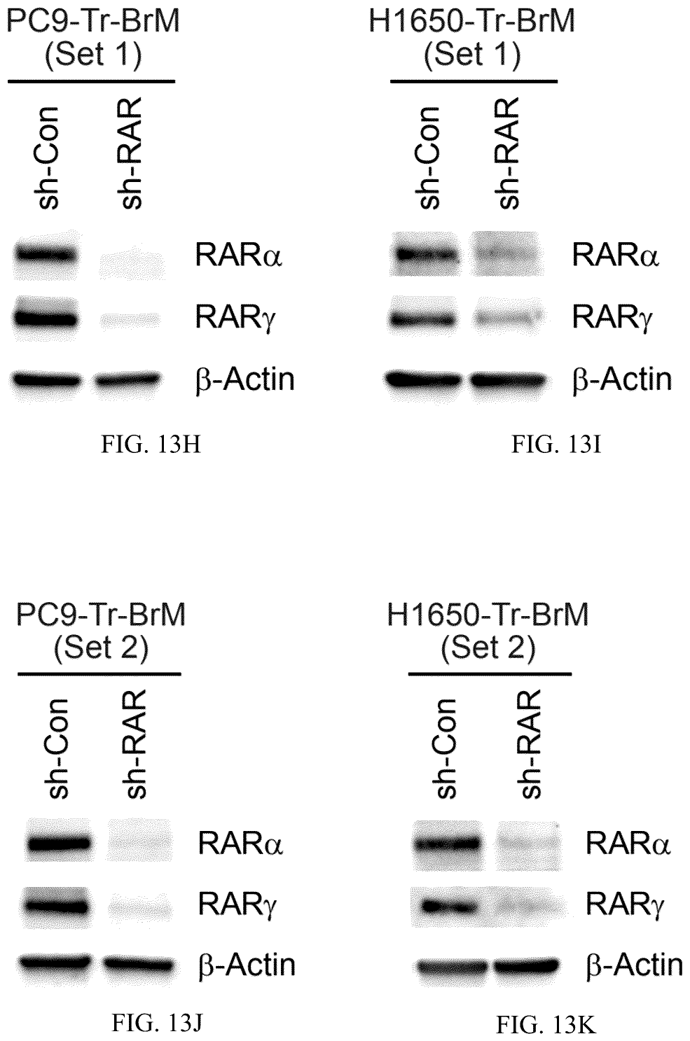

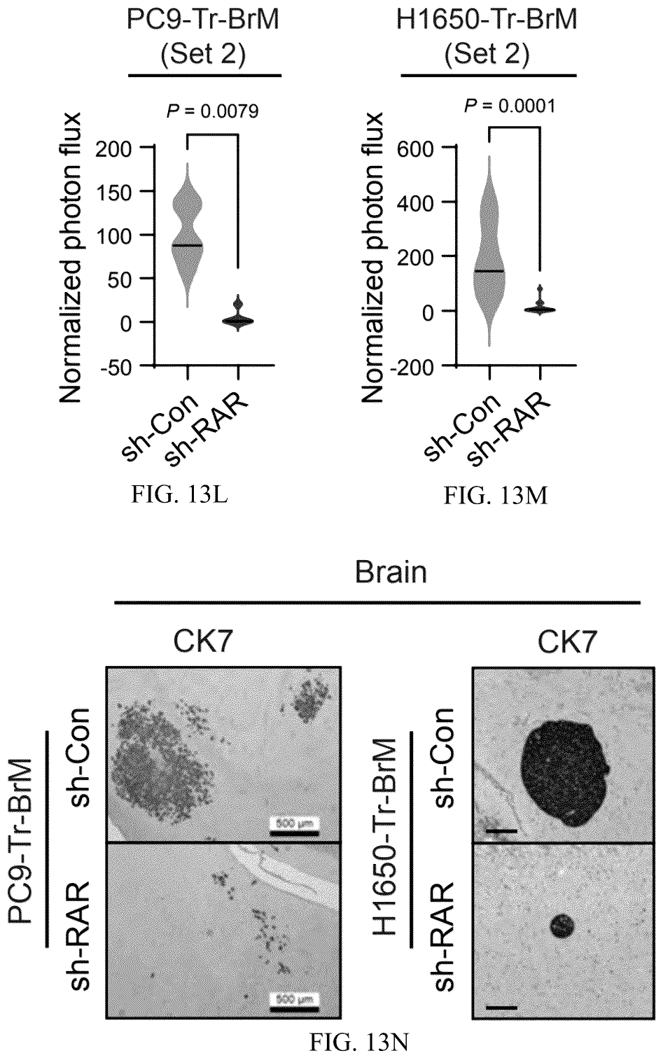

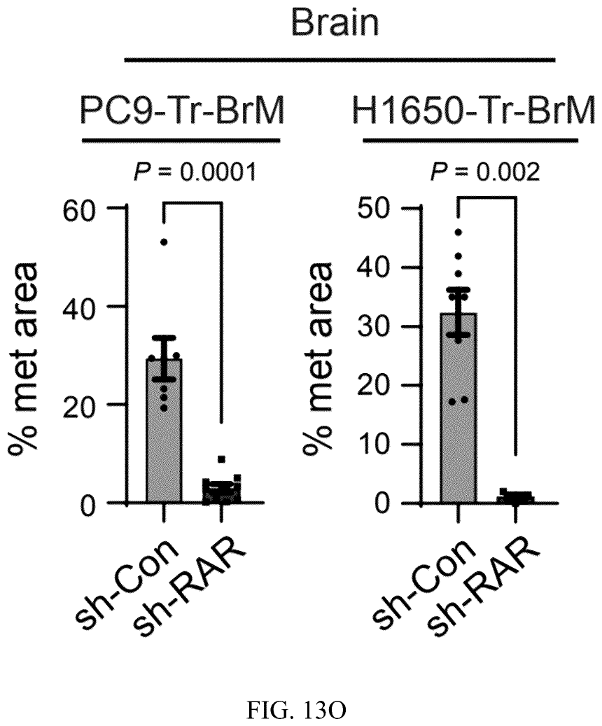

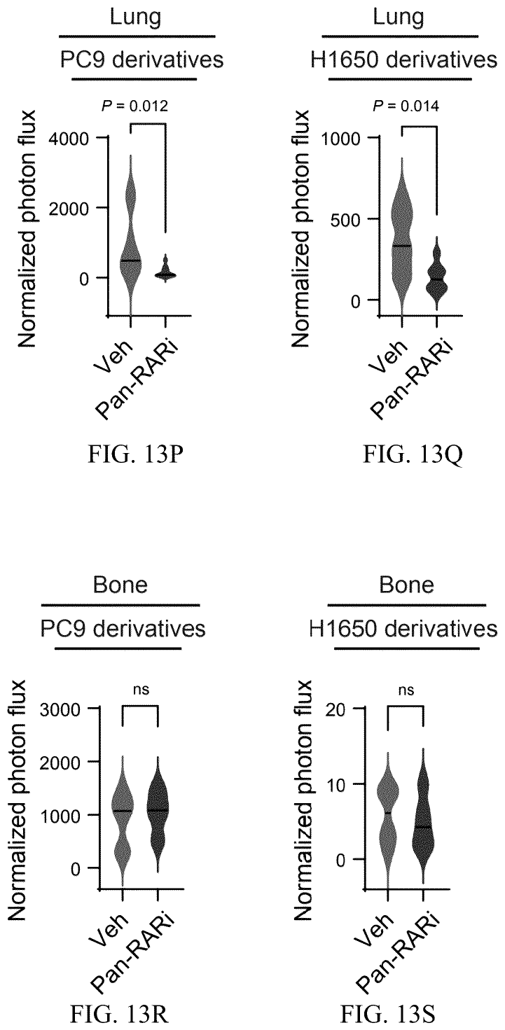

FIG. 2B: Brain sections from mice injected with PC9-BrM cells described in FIG. 1a-b were immunostained using an antibody against phospho-EGFR (Tyr1068). PC9-BrM cells were injected into the arterial circulation of immunodeficient mice via intracardiac injection to generate metastases. Treatment was administered starting at 25 days post tumor-cell injection with either vehicle or osimertinib (“Osi”) at 5 mg/kg body weight/day by oral gavage, 5 days per week, and continued until endpoint. The endpoint for vehicle-treated mice was two months post tumor-cell injection (“Vehicle”). The endpoint for osimertinib-treated mice was 4 months post tumor-cell injection for micrometastases (“Micromet”) and 8 months post tumor-cell injection for relapsed metastatic lesion (“Relapsed Met”). For the osimertinib treatment group, mice were administered osimertinib by oral gavage. At 6 hours post treatment, mice were euthanized, and brain tissues were subsequently processed for histological analysis. Representative images of immunohistochemical staining for phospho-EGFR in brain sections are shown. Arrows point to, and dotted line surrounds, the location of metastatic cells in the brain. Scale bars, 100 μm.

FIG. 2C: The pEGFR-immunostained brain sections described in panel (b) were quantitated using automated Qu-Path software to count pEGFR-positive cancer cells that were identified by setting a threshold for signal intensity (1+). Data are presented as mean values±SEM. P-values (indicated in the figures) were determined by a two-tailed, unpaired Mann-Whitney test: n=10 for vehicle-treated mice, 14 for osimertinib-treated mice with micrometastases, and 10 for osimertinib-treated mice with relapsed metastases.

FIG. 2D: Schematic representation of the strategies used to compare PC9-BrM and PC9-Tr-BrM cells for differentially expressed proteins by quantitative label-free mass spectrometry and for differentially expressed genes by transcriptomics.

FIG. 2E: Volcano plot shows the differentially expressed proteins between PC9-Tr-BrM and PC9-BrM cells identified by quantitative label-free mass spectrometry. Proteins with higher abundance in PC9-Tr-BrM cells compared to PC9-BrM cells have log 2 fold-changes with positive values and are labeled in red. N=3 replicates per group. Data points referring to the top significantly differentially expressed proteins (S100A9 and S100A8) are labeled.

FIG. 2F: Volcano plot of RNA-sequencing-based transcriptomic analysis shows the differentially expressed genes between PC9-Tr-BrM cells and PC9-BrM cells. Genes with significantly higher expression in PC9-Tr-BrM cells compared to PC9-BrM cells have log 2 fold-changes with positive values and are depicted in red. Genes with significantly lower expression in PC9-Tr-BrM cells compared to PC9-BrM cells have log 2 fold-changes with negative values and are depicted in grey. N=3 replicates per group. Data points referring to the top significantly differentially expressed genes (S100A9 and S100A8) are labeled.

FIG. 2G: Immunoblot analyses of lysates from BrM and Tr-BrM cells from both PC9- and H1650-derived models using antibodies against S100A9 and β-actin (loading control). The data are representative of three independent experiments.

FIG. 2H: S100A9 expression was determined by quantitative RT-PCR (qRT-PCR) analysis of PC9- and H1650-derived BrM and Tr-BrM cells. GAPDH was measured as an internal control. Data are presented as mean values±SEM. P-values were determined by a two-tailed, unpaired Mann-Whitney test: n=6 for PC9-BrM, n=6 for PC9-Tr-BrM; n=4 for H1650-BrM; n=5 for H1650-Tr-BrM.

FIG. 2I: Immunoblot analyses of lysates from PC9- and H1650-derived Tr-BrM cells infected with viruses expressing either a control guide-RNA (“Lenti-Con”) or a S100A9-specific guide-RNA (referred to as “S100A9i” throughout the figures). The indicated antibodies were used to confirm the loss of S100A9 protein expression following CRISPR-dCas9-mediated gene repression. β-actin served as a protein loading control. Data are representative of three independent experiments.

FIG. 2J: Ex-vivo photon flux of brains from mice injected with PC9- or H1650-derived Tr-BrM cells expressing either Lenti-con or S100A9i was determined by bioluminescence imaging. Mice were collected at 7 weeks post tumor-cell injection. The photon-flux scale is indicated on the right side.

FIG. 2K: Violin plots depicting normalized photon flux of brains imaged ex-vivo from the mice described in panel 2(j). The normalized photon flux for brain was calculated by dividing the photon flux from brain collected ex-vivo by the total photon flux at day 0 (i.e., the day of injection) and multiplying that value by 100. Data are presented as mean values±SEM. P-values were determined by a two-tailed, unpaired Mann-Whitney test. For PC9 Tr-BrM: n=10 for Lenti-Con and n=9 for S100A9i. For H1650-Tr-BrM: n=6 for Lenti-Con and n=7 for S100A9i.

FIG. 2L: Representative images of CK7 immunohistochemistry on brain sections from mice injected with either PC9-derived Tr-BrM Lenti-con-expressing (left panel) or S100A9i-expressing (right panel) cells in the top row, or H1650-derived Tr-BrM Lenti-Con-expressing (left panel) or S100A9i-expressing (right panel) cells in the bottom row. Brains were harvested from mice at 7 weeks post tumor-cell injection. Scale bars, 500 μm.

FIG. 2M: Quantitative analysis of the percentage of brain sections covered by metastasis from the experiment described in panel (1). Data are presented as mean values±SEM. P-values were determined by a two-tailed, unpaired Mann-Whitney test. For PC9-Tr-BrM, n=4 for Lenti-Con and n=4 for S100A9i. For H1650-Tr-BrM,

n=6 for Lenti-Con and n=3 for S100A9i.

### S100A9-Proficient Cells Promote Post-Colonization Growth in the Brain.

FIG. 3A: Schematic representation of the experimental design to quantify seeding in the brain. PC9-Tr-BrM cells expressing either lenti-control (“Lenti-Con”) or S100A9i were injected into the arterial circulation of immunodeficient mice via intracardiac injection. Seven days later, brains were isolated, sectioned and analyzed by immunohistochemistry for human CK7. CK7-immunostained cancer cells were then counted to compare seeding of cancer cells in the brain parenchyma between experimental groups.

FIG. 3B: Quantitative analysis of the experiment described in panel (a). Tumor cells were counted in 10 sections of 20 microns each per brain. Data are presented as mean values±SEM. The p-value was determined by a two-tailed, unpaired Mann-Whitney test. N=4 for Lenti-Con; n=5 for S100A9i.

FIG. 3C: Schematic representation of the experimental design to analyze post-colonization growth in the brain. PC9-Tr-BrM cells expressing either Lenti-Con or S100A9i were injected into the arterial circulation of immunodeficient mice via intracardiac injection. At 7 weeks post-injection, brain tissues were collected, and sections were analyzed by immunostaining for phospho-histone H3 (Ser10) to compare the number of mitotically active cancer cells between the experimental groups.

FIG. 3D: Representative images of phospho-histone-H3 (“p-Hist H3”) immunohistochemistry on brain sections from the experiment described in panel (c). Arrows point to, and dotted line surrounds, the location of metastatic cells in the brain. Scale bars, 100 μm.

FIG. 3E: Quantitative analysis of the phospho-histone-H3-positive cells within brain sections from the experiment described in panel (c) and represented in panel (d). Immunostained sections were counted using the Qu-Path software where positively stained cells are identified by setting a threshold for signal intensity (3+). Data are presented as mean values±SEM. P-values were determined by a two-tailed, unpaired Mann-Whitney test: n=12 for Lenti-Con, and n=6 for S100A9i.

FIG. 3F: Representative images of brain sections stained with an antibody against human S100A9. PC9-BrM cells were injected into the arterial circulation of immunodeficient mice via intracardiac injection. After metastatic signal was detected by bioluminescence imaging, treatment was started at 25 days post tumor-cell injection with either vehicle or osimertinib at 5 mg/kg body weight/day by oral gavage, 5 days per week. Brain tissues were collected at 2 months post tumor-cell injection in the vehicle treatment group (“Vehicle”), at 3 months post tumor cell-injection in the osimertinib treatment group (minimal residual disease or “MRD”), and at 8 months post tumor-cell injection in the osimertinib treated relapse group (“Relapse”). Scale bars, 100 μm. Data are representative of 10 mice/group analyzed at each timepoint.

FIG. 3G: Schematic representation of single-cell cloning from PC9-BrM cells. S100A9 high- and low-expressing single-cell progenies (SCPs) are labeled as S100A9^{high} and S100A9^{low}, respectively.

FIG. 3H: Immunoblot analysis of lysates from PC9-BrM-derived SCPs using antibodies against S100A8, S100A9, and β-actin (loading control). The data are representative of three independent experiments.

FIG. 3I: Schematic representation of the brain metastasis assay to compare the ability of S100A9^{hi} and S100A9^{low} SCPs to grow in the brain and generate metastases.

FIG. 3J: Ex-vivo photon flux of brains from mice injected with PC9-BrM-derived S100Ag^{hi} and S100A9^{low} SCPs was determined by bioluminescence imaging. Brains were collected from mice at 7 weeks post tumor-cell injection. Photon-flux scale is indicated below the images.

FIG. 3K: Violin plots depicting normalized photon flux of brains imaged ex-vivo from mice described in panel (j). The normalized photon flux for brain was calculated by dividing the photon flux from brain collected ex vivo by the total photon flux at day 0 (i.e., the day of injection) and multiplying that value by 100. Data are presented as mean values±SEM. P-values were determined by a two-tailed, unpaired Mann-Whitney test. N=5 for S100A9^{hi}; n=4 for S100A9^{low}.

FIG. 3L: Representative images of CK7 immunohistochemistry on brain sections from mice injected with PC9-BrM-derived S100A9^{hi} and S100A9^{low} SCPs. Brains were harvested from mice at 7 weeks post tumor-cell injection. Scale bars, 200 μm.

FIG. 3M: Quantitative analysis of the percentage of CK7-immunostained brain sections covered by metastasis in the experiment described in panel (1). Data are presented as mean values±SEM. P-values were determined by a two-tailed, unpaired Mann-Whitney test: n=5 for S100A9^{hi}, and n=5 for S100A9^{low}.

High S100A9 Expression is Associated with Brain Metastasis and Shorter PFS in Osimertinib-Treated Lung Cancer Patients.

FIG. 4A: Schematic representation of the analysis of patient samples for S100A9 expression in cancer cells. S100A9 immunostaining was performed on tissue specimens (biopsies/resected material) from 29 lung cancer patients with a validated EGFR mutation that were obtained prior to osimertinib treatment. The immunostained samples were scored by independent pathologists as either S100A9-positive (any percentage of clear, positive intracellular S100A9 staining in cancer cells) or S100A9-negative (no detectable S100A9 staining in cancer cells). “pos”, S100A9-positive; “neg”, S100A9-negative.

FIG. 4B: Graphical representation of the association between S100A9 expression in the patient tissue specimens described in panel (a) and the development of brain metastasis, for 26 patients with a clinical annotation for the presence or absence of brain metastasis at diagnosis (3 out of 29 patients had unknown brain metastasis status at diagnosis). The p-value was determined by a chi-squared test: n=10 samples from patients with brain metastasis, and n=16 samples from patients without brain metastasis.

FIG. 4C: Distribution of patients on first-, second- and third-line osimertinib treatment from the 29-patient cohort described in panel (a).

FIG. 4D: Kaplan-Meier plot for progression-free survival of osimertinib-treated patients from the combined cohort described in panel (a). Data were analyzed using the log-rank test: χ2=10.74, d.f.=1, p=0.0001, n=29 patients. Patients who had not progressed at the time of analysis were censored.

FIG. 4E: Kaplan-Meier plot for progression-free survival of the osimertinib-treated patients described in panels (a) and (c). First-line-osimertinib-treated patients are denoted as “first-line osi” (left), and combined second- and third-line osimertinib-treated patients are denoted as “second/third-line osi” (right). Data were analyzed using the log-rank test. For first-line osimertinib-treated patients: χ2=6.011, d.f.=1, p=0.0106, n=17; for second- and third-line osimertinib-treated patients: χ2=4.015, d.f.=1, p=0.0451, n=12. Patients who had not progressed at the time of analysis were censored.

### S100A9 Promotes Brain Relapse Through ALDH1A1.

FIG. 5A: Volcano plot shows the significantly differentially expressed genes between PC9-Tr-BrM cells expressing either Lenti-con or S100A9i guide-RNAs as identified by RNA-sequencing analysis. Genes with significantly higher expression in PC9-Tr-BrM-S100A9i cells compared to PC9-Tr-BrM-Lenti-Con cells have log 2 fold-changes with positive values and are depicted in red. Genes with significantly lower expression in PC9-Tr-BrM-S100A9i cells compared to PC9-Tr-BrM-Lenti-Con cells have log 2 fold-changes with negative values and are depicted in blue. N=3 per group. Genes with an adjusted p-value of less than 1.0×10⁻⁴ and an absolute value of the log 2 fold change of greater than 2.4 were considered significant.

FIG. 5B: Heatmap of the top significantly up- and down-regulated genes in PC9-Tr-BrM-derived S100A9i-expressing cells versus Lenti-Con-expressing cells (“Con”). Normalized gene expression above the row mean is indicated by progressively darker shades of red, and normalized gene expression below the row mean is indicated by progressively darker shades of blue. Genes with an adjusted p-value of less than 1.0×10⁻⁴ and an absolute value of the log 2 fold change of greater than 2.4 were considered significant.

FIG. 5C: ALDH1A1 expression was determined by qRT-PCR analysis in PC9-derived Tr-BrM cells expressing either Lenti-Con or S100A9i. GAPDH was measured as an internal control. Data are presented as mean values±SEM. The p-value was determined by a two-tailed, unpaired Mann-Whitney test: n=6 for Lenti-Con, and n=6 for S100A9i.

FIG. 5D: Immunoblot analysis of lysates from PC9-Tr-BrM cells expressing either Lenti-Con or S100A9i using antibodies against ALDH1A1 and β-actin (loading control). The data are representative of three independent experiments.

FIG. 5E: Representative images of serial brain sections stained with an antibody against S100A9 (left panel) and ALDH1A1 (right panel) taken from two different mice (designated “Tr-BrM 1” and “Tr-BrM 2”) injected with PC9-Tr-BrM cells. Brains were collected at 7 weeks post tumor-cell injection. Images are representative of 8 mice analyzed per group. Scale bars, 2000 μm.

FIG. 5F: Representative images of brain sections stained with an antibody against ALDH1A1 taken from mice injected with PC9-Tr-BrM cells expressing either Lenti-Con or S100A9i and collected at 7 weeks post tumor-cell injection. Scale bars, 200 μm.

FIG. 5G: Quantitative analysis of the ALDH1A1-positive cells shown in panel (f). Immunostained sections were counted using the Qu-Path software where positively stained cells were identified by setting a threshold for signal intensity (1+). Data are presented as mean values±SEM. The p-value was determined by a two-tailed, unpaired Mann-Whitney test: n=5 for Lenti-Con, and n=5 for S100A9i.

FIG. 5H: Immunoblot analysis of lysates from PC9-Tr-BrM cells expressing either Lenti-Con or ALDH1A1i using antibodies against ALDH1A1 and β-actin (loading control). The data are representative of three independent experiments.

FIG. 5I: Ex-vivo photon flux of brains from mice injected with PC9-Tr-BrM-derived cells expressing either Lenti-Con or ALDH1A1i was determined by bioluminescence imaging. Brains were collected from mice at 7 weeks post tumor-cell injection. Photon-flux scale is indicated on the right side.

FIG. 5J: Violin plots depicting normalized photon flux of brains imaged ex-vivo from the mice described in panel 5(i). The normalized photon flux for brain was calculated by dividing the photon flux from brain collected ex-vivo by the total photon flux at day 0 (i.e., the day of injection) and multiplying that value by 100. Data are presented as mean values±SEM. The p-value was determined by a two-tailed, unpaired Mann-Whitney test: n=5 for Lenti-Con, and n=5 for ALDH1A1i.

FIG. 5K: Representative images from CK7 immunohistochemistry on brain sections from mice injected with PC9-Tr-BrM-Lenti-Con cells (top) or PC9-Tr-BrM-ALDH1A1i cells (bottom). Brains were harvested from mice at 7 weeks post tumor-cell injection. Scale bars, 200 μm.

FIG. 5L: Quantitative analysis of the percentage of CK7-immunostained brain sections covered by metastasis from the experiment described in panel (k). Data are presented as mean values±SEM. The p-value was determined by a two-tailed, unpaired Mann-Whitney test: n=5 for Lenti-Con, and n=3 for ALDH1A1i.

FIG. 5M: Immunoblot analysis of lysates from PC9-Tr-BrM-S100A9i cells expressing either lenti-vector control (“Lenti Vec-Con”) or ALDH1A1 (“ALDH1A1 o/e” denotes ALDH1A1 overexpression) using antibodies against ALDH1A1 and β-actin (loading control). Data are representative of three independent experiments.

FIG. 5N: Ex-vivo photon flux of brains from mice injected with PC9-Tr-BrM-S100A9i cells expressing either Lenti-Vec Con or ALDH1A1 was determined by bioluminescence imaging. Brains were collected from mice at 7 weeks post tumor-cell injection. The photon-flux scale is indicated on the right side.

FIG. 5O: Violin plots depicting normalized photon flux of brains imaged ex-vivo from the mice described in panel (n). The normalized photon flux for brain was calculated by dividing the photon flux from brain collected ex-vivo by the total photon flux at day 0 (i.e., the day of injection) and multiplying that value by 100. Data are presented as mean values±SEM. The p-value was determined by a two-tailed, unpaired Mann-Whitney test: n=6 for Lenti-Vec Con; n=5 for ALDH1A1 o/e.

FIG. 5P: Representative images of CK7 immunohistochemistry on brain sections from mice injected with PC9-Tr-BrM S100A9i cells expressing either Lenti-Vec Con (top) or ALDH1A1 (bottom). Brains were harvested from mice at 7 weeks post tumor-cell injection. Scale bars, 200 μm.

FIG. 5Q: Quantitative analysis of the percentage of CK7-immunostained brain sections covered by metastasis from the experiment described in panel (p). Data are presented as mean values±SEM. The p-value was determined by a two-tailed, unpaired Mann-Whitney test: n=5 for Lenti-Vec Con, and n=6 for ALDH1A1 o/e.

### Osimertinib-Refractory Cancer Cells are Sensitive to Pan-RAR Inhibition.

FIG. 6A: Schematic representation of the treatment of PC9- and H1650-derived Tr-BrM cells with vehicle, osimertinib alone, a pan-RAR antagonist (AGN194310) alone or AGN194310 in combination with osimertinib. At 25 days post tumor-cell injection, after confirmation of metastatic signal, mice were administered long-term treatment with either 1) vehicle (“Veh”), 2) AGN194310 (“Pan-RARi”, 0.5 mg/kg body weight/day), 3) osimertinib (“Osi”, 5 mg/kg body weight/day) or 4) AGN194310 (0.5 mg/kg body weight/day) plus osimertinib (5 mg/kg body weight/day) by oral gavage, 5 days per week, until the endpoint (7 weeks post tumor-cell injection).

FIG. 6B: Ex-vivo photon flux of post-treatment brains from the experiment described in panel 6(a) was determined at endpoint by bioluminescence imaging. The photon-flux scale is indicated on the right side.

FIG. 6C: Violin plots depicting normalized photon flux of brains imaged ex-vivo from the mice described in panel 6(b). The normalized photon flux for brain was calculated by dividing the photon flux from brain collected ex-vivo by the total photon flux at day 0 (i.e., the day of injection) and multiplying that value by 100. Data are presented as mean values±SEM. P-values were determined by a two-tailed, unpaired Mann-Whitney test. For PC9-Tr-BrM mice: n=8 for vehicle; n=8 for osimertinib; n=7 for pan-RARi; and n=7 for osimertinib plus pan-RARi. For H1650-Tr-BrM mice: n=12 for vehicle; n=8 for osimertinib; n=10 for pan-RARi; and n=6 for osimertinib plus pan-RARi.

FIG. 6D: Representative images of CK7 immunohistochemistry on post-treatment brain sections collected at endpoint from the experiment described in panel (a). Scale bars, 200 μm for upper panel (PC9-Tr-BrM) and 100 μm for lower panel (H1650-Tr-BrM).

FIG. 6E: Quantitative analysis of the percentage of CK7-immunostained brain sections covered by metastasis shown in panel (d). Data is presented as mean values±SEM. P-values were determined by a two-tailed, unpaired Mann-Whitney test. For PC9-Tr-BrM mice: n=11 for vehicle; n=9 for osimertinib; n=4 for pan-RARi; n=5 for osimertinib plus pan-RARi. For H1650-Tr-BrM mice: n=6 for vehicle; n=6 for osimertinib; n=9 for pan-RARi; and n=6 for osimertinib plus pan-RARi.

FIG. 6F: Schematic representation of experiment testing the effect of RAR gene knockdown on brain metastasis development. Mice were injected with either PC9- or H1650-derived Tr-BrM cells with one of two sets of shRNA-mediated stable dual knockdown of RAR□ and RAR□ (“sh-RARγ+α”), or control shRNA (“sh-Con”), via intracardiac injection. Experiments involving shRNA set #1 are shown in this figure in panels 6g-j while experiments involving shRNA set #2 are shown in FIG. 13l-o. Mice were euthanized at 7 weeks post tumor-cell injection, and brains were collected for analysis.

FIG. 6G: Ex-vivo photon flux of post-treatment brains from the experiment described in panel 6(f) was determined at endpoint by bioluminescence imaging. The photon-flux scale is indicated on the right side.

FIG. 6H: Violin plots depicting normalized photon flux of brains imaged ex-vivo from the mice represented in panel (g). The normalized photon flux for brain was calculated by dividing the photon flux from brain collected ex-vivo by the total photon flux at day 0 (i.e., the day of injection) and multiplying that value by 100. Data are presented as mean values±SEM. P-values were determined by a two-tailed, unpaired Mann-Whitney test. For PC9-Tr-BrM, n=7 for sh-Con; n=5 for sh-RAR. For H1650-Tr-BrM: n=8 for sh-Con; n=6 for sh-RAR.

FIG. 6I: Representative images of CK7 immunohistochemistry on post-treatment brain sections collected at endpoint from the experiment described in panel (f). Scale bars, 100 μm for all images.

FIG. 6J: Quantitative analysis of the percentage of CK7-immunostained brain sections covered by metastasis shown in (i). Data is presented as mean values±SEM. P-values were determined by a two-tailed, unpaired Mann-Whitney test. For PC9-Tr-BrM, n=10 for sh-Con; n=10 for sh-RAR. For H1650-Tr-BrM: n=5 for sh-Con; n=5 for sh-RAR.

### The Combination of Osimertinib and Pan-RAR Antagonism Reduces Residual Cancer Cells in the Brain.

FIG. 7A: Schematic representation of the experimental treatment protocol for the prevention trial. Osimertinib-sensitive PC9- or H1650-BrM cells were injected into mice via intracardiac injections. At 5 days post tumor-cell injection, mice were administered treatment with either 1) vehicle, 2) AGN-194310 (0.5 mg/kg body weight/day), 3) osimertinib (5 mg/kg body weight/day), or 4) AGN-194310 (0.5 mg/kg body weight/day) plus osimertinib (5 mg/kg body weight/day) by oral gavage, 5 days per week, until the endpoint (7 weeks post tumor-cell injection).

FIG. 7B: Representative images of CK7 immunohistochemistry on post-treatment brain sections at endpoint from the experiment described in panel (a). Scale bars, 500 μm for PC9-BrM and 100 μm for H1650-BrM.

FIG. 7C: Quantitative analysis of the CK7-immunostained brain metastatic cancer cell number per μm² represented in panel (b). Data is presented as the mean number of cancer cells per μm² of the brain tissue section±SEM. P-values were determined by a two-tailed, unpaired Mann-Whitney test. For PC9-BrM: n=12 for vehicle; n=16 for osimertinib; n=10 for pan-RARi; n=10 for osimertinib plus pan-RARi. For H1650-BrM: n=17 for vehicle; n=12 for osimertinib; n=10 for pan-RARi; and n=14 for osimertinib plus pan-RARi.

Fatal Brain Relapse in Osimertinib-Treated, Metastatic Lung Cancer Mouse Models. [Supplementary FIG. 1¹]¹FIGS. 8-13 correspond to the “supplementary figures” 1-6 associated with the publication, Biswas, A. K., et al., “Targeting S100A9-ALDH1A1-Retinoic Acid Signaling to Suppress Brain Relapse in EGFR-Mutant Lung Cancer,” Cancer Discovery, Vol. 12(4), pp. 1002-1021 (Apr. 1, 2022), the contents of which are hereby incorporated by reference in their entirety. This publication is cited herein as “Biswas, Cancer Discovery.”

FIG. 8A: Quantitation of photon flux of brain versus rest of the body (representing extracranial sites such as bone and lymph nodes) by longitudinal imaging in mice from the experiments described in FIG. 1a-c involving the PC9-derived model. Days represent days after initial tumor-cell injection. Data is normalized to day 25 (the initial day of treatment). N=4 for vehicle-treated mice, and n=4 for osimertinib-treated mice. Data are representative of three independent experiments.

FIG. 8B: Schematic representing the generation of brain-metastatic-derivative (BrM) cells from the EGFR-mutant H1650 parental cell line by the in-vivo selection method.

FIG. 8C: Representative image of ex-vivo bioluminescence imaging of a brain collected from a mouse injected with the H1650 parental cell line (to generate the H1650 BrM line as described in panel (b)). The photon-flux scale is indicated on the right side. The data are representative of three independent experiments. The percentage of mice (n=15) with brain and lung lesions is shown.

FIG. 8D: Schematic representation of the human EGFR-mutant H1650-BrM in-vivo treatment model for metastatic EGFR-mutant lung cancer. Luciferase-labeled H1650-BrM cells were injected into the arterial circulation of immunodeficient mice via intracardiac injection to generate metastases, which were detected by bioluminescence imaging. At 25 days post tumor-cell injection, after confirmation of metastatic signal, mice were administered long-term treatment with either vehicle or osimertinib at 5 mg/kg body weight/day by oral gavage, 5 days per week, until the endpoint, which averaged to 4 months post tumor-cell injection.

FIG. 8E: Kaplan-Meier plot for brain-metastasis-progression-free survival of mice injected with H1650-BrM cells followed by treatment with either vehicle (“Veh”) or osimertinib (“Osi”). Days elapsed represent days after initial tumor-cell injection. Data were analyzed using the log-rank test: χ2=11.42, d.f.=1, p=0.0007, n=5 for vehicle-treated mice, and n=6 for osimertinib-treated mice. The percentage of mice (n=20) with brain and lung lesions post-osimertinib treatment is shown from multiple experiments.

FIG. 8F: Kaplan-Meier plot for brain-metastasis-progression-free survival of mice injected with H1650-Tr-BrM cells followed by treatment with either vehicle or osimertinib. Data were analyzed using the log-rank test: χ2=2.541, d.f.=1, p-value not significant (ns), n=6 for vehicle-treated mice, and n=4 for osimertinib-treated mice.

FIG. 8G: Representative images of human cytokeratin 7 (CK7) immunohistochemistry in brain sections from mice injected with either H1650-BrM (upper panel) or H1650-Tr-BrM cells (lower panel) and treated with either vehicle (left panel) or osimertinib (right panel). Arrow points to the location of metastatic cells in the brain. Mice were euthanized at 60 days post tumor-cell injection. Scale bars, 200 μm.

FIG. 8H: Quantitative analysis of the percentage of brain tissue sections covered by metastasis from the experiment described in panel (g). Data are presented as mean values±SEM. P-values were determined by a two-tailed, unpaired Mann-Whitney test. For H1650-BrM, n=5 for vehicle and n=4 for osimertinib; for H1650-Tr-BrM, n=5 for vehicle and n=4 for osimertinib.

### Enrichment of S100A8/9 Proteins in Osimertinib-Refractory Cancer Cells.

FIG. 9A: Immunoblot analysis for EGFR pathway activation in H1650-BrM and H1650-Tr-BrM cells treated with the indicated doses of osimertinib and collected at 6 hours post treatment. Antibodies against phospho-EGFR (Tyr1068), phospho-ERK, total-EGFR, total-ERK and β-actin (loading control) were used. Data are representative of three independent experiments.

FIG. 9B: In-vitro cell viability assay for comparing osimertinib sensitivity between PC9-BrM and PC9-Tr-BrM derivatives. Cell viability values are normalized to the vehicle (DMSO)-treated control, presented as mean values±SEM, and representative of three independent experiments.

FIG. 9C: In-vitro cell viability assay for comparing osimertinib-sensitivity between H1650-BrM and H1650-Tr-BrM derivatives. Cell viability values are normalized to the vehicle (DMSO)-treated control, presented as mean values±SEM, and representative of three independent experiments.

FIG. 9D: Brain sections from mice injected with H1650-BrM cells described in FIG. 8g-h were immunostained using an antibody against phospho-EGFR (Tyr1068). H1650-BrM cells were injected into the arterial circulation of immunodeficient mice via intracardiac injection to generate metastases. Treatment was started at 25 days post tumor-cell injection with either vehicle or osimertinib at 5 mg/kg body weight/day by oral gavage, administered 5 days per week, and continued until endpoint at two months post tumor-cell injection. Mice were then euthanized, and brain tissues were subsequently processed for histological analysis. Representative images of immunohistochemical staining for phospho-EGFR in brain sections are shown. Dotted lines surround the location of metastatic cells in the brain. Scale bars, 100 μm.

FIG. 9E: The pEGFR-stained brain sections from the H1650-derived model described in panel (d) were quantitated using automated Qu-Path software to count pEGFR-positive cancer cells that were identified by setting a threshold for signal intensity (1+). Data are represented as mean values±SEM. The p-value was determined by a two-tailed, unpaired Mann-Whitney test: n=5 for vehicle-treated mice and n=5 for osimertinib-treated mice.

FIG. 9F: Heatmap of the top 25 significantly up- and down-regulated genes in PC9-Tr-BrM cells compared to PC9-BrM cells. Normalized gene expression above the row mean is indicated by progressively darker shades of red, and normalized gene expression below the row mean is indicated by progressively darker shades of blue. Genes with an adjusted p-value of less than 1.0×10-4 and an absolute value of the log 2 fold change of greater than 2.4 were considered significant.

FIG. 9G: Heatmap of the top significantly up-regulated (2133) and down-regulated (2460) genes in H1650-Tr-BrM versus H1650-BrM cells is shown. Normalized gene expression above the row mean is indicated by progressively darker shades of red, and normalized gene expression below the row mean is indicated by progressively darker shades of blue. Genes with an adjusted p-value of less than 1.0×10-4 and an absolute value of the log 2 fold change of greater than 0.5 were considered significant. Approximate positions of the indicated genes are shown in the heatmap.

FIG. 9H: GO terms identified as significant and convergent via GO-term analysis of the top 300 up-regulated genes in PC9- and H1650-Tr-BrM lines compared to their respective controls by G: Profiler analysis.

FIG. 9I: GO terms identified as significant and convergent via GO-term analysis of the top 300 down-regulated genes in PC9- and H1650-Tr-BrM lines compared to their respective controls by G: Profiler analysis.

### S100A8/9 as Key Mediators of Brain Relapse in Osimertinib-Refractory Lung Cancer Cells.

FIG. 10A: Immunoblot analysis of lysates from BrM and Tr-BrM cells from both PC9- and H1650-derived models using antibodies against S100A8 and β-actin (loading control). Images are representative of three independent experiments.

FIG. 10B: S100A8 expression was determined by quantitative RT-PCR (qRT-PCR) analysis in PC9- and H1650-derived BrM and Tr-BrM cells. GAPDH was used as an internal control. Data are presented as mean values±SEM. P-values were determined by a two-tailed, unpaired Mann-Whitney test. For PC9, n=6 for BrM, and n=6 for Tr-BrM. For H1650, n=5 for BrM, and n=5 for Tr-BrM.1

FIG. 10C-FIG. 10D: Representative images of brain sections stained with an antibody against human S100A9 from mice bearing brain metastases when injected with PC9-Tr-BrM cells (10(c)) and H1650-Tr-BrM cells (10(d)) and collected 7 weeks post tumor-cell injection. Scale bar, 100 μm.

FIG. 10E: S100A9 expression was determined by qRT-PCR analysis in PC9-Tr-BrM cells expressing either lenti-control (“Lenti-Con”) or S100A9i. GAPDH was used as an internal control. Data are presented as mean values±SEM. The p-value was determined by a two-tailed, unpaired Mann-Whitney test, n=5 for Lenti-Con, and n=6 for S100A9i.

FIG. 10F: S100A9 expression was determined by qRT-PCR analysis in H1650-Tr-BrM cells expressing either LentiCon or S100A9i. GAPDH was used as an internal control. Data are presented as mean values±SEM. The p-value was determined by a two-tailed, unpaired Mann-Whitney test, n=6 for Lenti-Con and n=6 for S100A9i.

FIG. 10G: S100A9 expression was determined by qRT-PCR analysis in PC9-Tr-BrM cells expressing either Lenti-Con or a second guide-RNA for S100A9i (referred to herein as “S100A9i (gRNA2)”). GAPDH was used as an internal control. Data are presented as mean values±SEM. The p-value was determined by a two-tailed, unpaired Mann-Whitney test, n=6 for Lenti-Con and n=6 for S100A9i-gRNA2.

FIG. 10H: S100A8 expression was determined by qRT-PCR analysis in PC9-Tr-BrM cells expressing either Lenti-Con or S100A8i. GAPDH was used as an internal control. Data are presented as mean values±SEM. The p-value was determined by a two-tailed, unpaired Mann-Whitney test: n=5 for Lenti-Con, and n=5 for S100A8i.

FIG. 10I: Immunoblot analyses of lysates from PC9- and H1650-derived Tr-BrM cells expressing either Lenti-Con or S100A8i using antibodies against S100A8 and β-actin (loading control). Data is representative of three independent experiments.

FIG. 10J: Ex-vivo photon flux of brains from mice injected with either PC9- or H1650-derived Tr-BrM cells expressing either Lenti-Con or S100A8i was determined by bioluminescence imaging. Mice were collected at 7 weeks post tumor-cell injection. The photon-flux scale is indicated on the right side of the image.

FIG. 10K: Violin plots depicting the normalized photon flux of brains imaged ex-vivo that are represented in panel 10(j). The normalized photon flux for brain was calculated by dividing the photon flux from brain collected ex-vivo by the total photon flux at day 0 (i.e., the day of injection) and multiplying that value by 100. Data are presented as mean values±SEM. N=6 mice for PC9-Tr-BrM-Lenti-Con, n=3 for PC9-Tr-BrM-S100A8i, n=5 per group for both H1650-Tr-BrM-Lenti-Con and H1650-Tr-BrM-S100A8i. The p-values were determined by a two-tailed, unpaired Mann-Whitney test.

FIG. 10L: Representative images of CK7 immunohistochemistry in brain sections from mice injected with either PC9- or H1650-derived Tr-BrM cells expressing either Lenti-Con or S100A8i. Mice were harvested at 7 weeks post tumor-cell injection. Scale bars, 200 μm.

FIG. 10M: Quantitative analysis of the percentage of CK7-immunostained brain sections covered by metastasis that are represented in panel (1). Data are presented as mean values±SEM. P-values were determined by a twotailed, unpaired Mann-Whitney test. N=5 per group for both PC9 and H1650-derived Tr-BrM-Lenti-Con and PC9-Tr-BrM-S100A8i.

FIG. 10N: Representative images of CK7 immunohistochemistry in brain sections from mice injected with PC9-TrBrM-Lenti-Con cells or PC9-Tr-BrM-S100A9i-gRNA2 cells. Mice were harvested at 7 weeks post tumor-cell injection. Scale bars, 100 μm.

FIG. 10O: Quantitative analysis of the percentage of CK7-immunostained brain sections covered by metastasis from the experiment represented in panel (n). Data are presented as mean values±SEM. The p-value was determined by a two-tailed, unpaired Mann-Whitney test: n=5 for PC9-Tr-BrM-Lenti-Con, and n=3 for PC9-Tr-BrM-S100A9i-gRNA2.

FIG. 10P: S100A8 expression was determined by qRT-PCR analysis in PC9-Tr-BrM cells expressing either LentiCon, S100A9i (left graph) or S100A9i-gRNA2 (right graph). GAPDH was used as an internal control. Data are presented as mean values±SEM. P-values were determined by a two-tailed, unpaired Mann-Whitney test. For the left graph, n=4 for PC9-Tr-BrM-Lenti-Con, and n=5 for PC9-Tr-BrM S100A9i. For the right graph, n=5 for PC9-Tr-BrM-Lenti-Con and n=6 for PC9-Tr-BrM-S100A9i-gRNA2.

FIG. 10Q: Immunoblot analysis of lysates from PC9- and H1650-derived Tr-BrM cells expressing either Lenti-Con or S100A9i using antibodies against S100A8 and β-actin (loading control). Data are representative of three independent experiments.

FIG. 10R: In-vitro cell proliferation assay comparing PC9-BrM and PC9-Tr-BrM cells. Data are presented as the mean cell number±SEM and are representative of three independent experiments.

FIG. 10S: In-vitro cell proliferation assay comparing H1650-BrM and H1650-Tr-BrM cells. Data are presented as the mean cell number±SEM and are representative of three independent experiments.

FIG. 10T: In-vitro cell proliferation assay comparing PC9-Tr-BrM-Lenti-Con and PC9-Tr-BrM-S100A9i cells. Data are presented as the mean cell number±SEM and are representative of three independent experiments.

FIG. 10U: In-vitro cell proliferation assay comparing H1650-Tr-BrM-Lenti-Con and H1650-Tr-BrM-S100A9i cells. Data are presented as the mean cell number±SEM and are representative of three independent experiments.

### Down-Regulation of ALDH1A1 in Brain Metastatic Cells Upon S100A9 Repression.

FIG. 11A: Heatmap of the top 855 significantly up-regulated (473) and down-regulated (382) genes in H1650-Tr-BrMderived S100A9i cells versus Lenti-Control cells (“Control”). Normalized gene expression above the row mean is indicated by progressively darker shades of red, and normalized gene expression below the row mean is indicated by progressively darker shades of blue. Genes with an adjusted p-value of less than 1.0×10-3 and an absolute value of the log 2 fold change of greater than 1.5 were considered significant. Approximate positions of the indicated genes in the heatmap are shown.

FIG. 11B: GO terms identified as significant and convergent via GO-term analysis of the top 300 down-regulated genes upon S100A9 repression in the PC9- and H1650-derived Tr-BrM cell lines by G: Profiler Analysis.

FIG. 11C: GO terms identified as significant and convergent via GO-term analysis of the top 300 up-regulated genes upon S100A9 repression in the PC9- and H1650-derived cell lines by G: Profiler Analysis.

FIG. 11D-FIG. 11E: Gene set enrichment plots for GOBP Retinoic Acid Metabolism Process and KEGG Retinol Metabolism signatures from the RNA-seq expression profiling data comparing PC9-Tr-BrM-S100A9i to PC9-Tr-BrM-LentiCon cells in FIG. 5b. Genes are ranked according to their expression in PC9-Tr-BrM-S100A9i cells from left to right. A positive normalized enrichment score (NES) indicates signature enrichment in PC9-Tr-BrM-S100A9i cells, and a negative normalized enrichment score indicates signature enrichment in PC9-Tr-BrM-Lenti-Con cells.

FIG. 11F-FIG. 11G: Heatmaps of key retinoic acid metabolism genes in the PC9-BrM versus PC9-Tr-BrM RNA-seq datasets from FIG. 9f and in the PC9-Tr-BrM-Lenti-Con versus PC9-Tr-BrM-S100A9i datasets from FIG. 5b. Shown here are the genes comprising the combined leading edges from both the GOBP Retinoic Acid Metabolism Process and the KEGG Retinol Metabolism gene sets in both the PC9-BrM-versus-PC9-Tr-BrM and the PC9-Tr-BrM-Lenti-Con-versus-PC9-Tr-BrM-S100A9i RNA-seq datasets. All leading-edge genes with detectable expression levels in the respective RNA-seq datasets were included in both (f) and (g). Normalized gene expression above the row mean is indicated by progressively darker shades of red, and normalized gene expression below the row mean is indicated by progressively darker shades of blue.

FIG. 11H: Boxplots of normalized gene expression of ALDHA1, ALDHA12 and ALDH1A3 in both PC9- and H1650-derived Tr-BrM-Lenti-Con and Tr-BrM-S100A9i cells (PC9 model shown as log 2-normalized expression and H1650 as normalized expression). Horizontal lines indicate the 25th and 75th quartiles and the median, and whiskers indicate the minimum and maximum values. P-values represent the adjusted p-value (Wald test) for the differences in expression of each gene between PC9- and H1650-derived Tr-BrM-S100A9i and Tr-BrM-Lenti-Con cells.

FIG. 11I: ALDH1A1 expression was determined by qRT-PCR analysis in PC9-Tr-BrM cells expressing either Lenti-Con or S100A9i-gRNA2. GAPDH was used as an internal control. Data are presented as mean values±SEM. The p-value was determined by a two-tailed, unpaired Mann-Whitney test, n=6 for Lenti-Con, and n=6 for S100A9i-gRNA2.

FIG. 11J: ALDH1A1 expression was determined by qRT-PCR analysis in H1650-Tr-BrM cells expressing either Lenti-Con or S100A9i. GAPDH was used as an internal control. Data are presented as mean values±SEM. The p-value was determined by a two-tailed, unpaired Mann-Whitney test, n=4 for Lenti-Con and n=4 for S100A9i.

FIG. 11K: Quantitative analysis of the ALDH1A1-positive cells from brain sections (that were stained with an antibody against ALDH1A1) taken from mice injected with H1650-Tr-BrM cells expressing either Lenti-Con or S100A9i and collected at 7 weeks post tumor-cell injection. Immunostained sections were counted using the Qu-Path software where positively stained cells were identified by setting a threshold for signal intensity (1+). Data are presented as mean values±SEM. The p-value was determined by a two-tailed, unpaired Mann-Whitney test: n=4 for Lenti-Con, and n=4 for S100A9i.

FIG. 11L: ALDH1A1 expression was determined by qRT-PCR analysis in H1650-Tr-BrM cells expressing either Lenti-Con or ALDH1A1i. GAPDH was used as an internal control. Data are presented as mean values±SEM. The p-value was determined by a two-tailed, unpaired Mann-Whitney test: n=4 for Lenti-Con, and n=4 for ALDH1A1i.

FIG. 11M: Ex-vivo photon flux of brains from mice injected with H1650-Tr-BrM cells expressing either Lenti-Con or ALDH1A1i was determined by bioluminescence imaging. Mice were collected at 7 weeks post tumor-cell injection. The photon flux scale is indicated on the right side of the image.

FIG. 11N: Violin plots depicting normalized photon flux of brains imaged ex-vivo from the mice described in panel (m). The normalized photon flux for brain was calculated by dividing the photon flux from brain collected ex-vivo by the total photon flux at day 0 (i.e., the day of injection) and multiplying that value by 100. Data are presented as mean values±SEM. P-values were determined by a two-tailed, unpaired Mann-Whitney test: n=6 for H1650-Tr-BrM-Lenti-Con, and n=7 for H1650-Tr-BrM-ALDH1A1i.

FIG. 11O: Representative images of CK7 immunohistochemistry in brain sections from mice injected with H1650-derived Tr-BrM cells expressing either Lenti-Con or ALDH1A1i. Mice were euthanized at 7 weeks post tumor-cell injection. Scale bars, 200 μm.

FIG. 11P: Quantitative analysis of the percentage of CK7-immunostained brain sections covered by metastasis that are represented in panel (o). Data are presented as mean values±SEM. P-values were determined by a two-tailed, unpaired Mann-Whitney test: n=5 for H1650-Tr-BrM-Lenti-Con, and n=5 for H1650-Tr-BrMALDH1A1i.

FIG. 11Q: ALDH1A1 expression was determined by qRT-PCR analysis in PC9-Tr-BrM-S100A9i cells expressing either lenti-vector control (“Lenti-Vec Con”) or ALDH1A1 cDNA (“ALDH1A1 o/e”). GAPDH was used as an internal control. Data are presented as mean values±SEM. The p-value was determined by a two-tailed, unpaired Mann-Whitney test, n=9 for Lenti-Vec Con, and n=3 for ALDH1A1 o/e.

FIG. 11R: ALDH1A1 expression was determined by qRT-PCR analysis in H1650-Tr-BrM-S100A9i cells expressing either Lenti-Vec Con or ALDH1A1 o/e. GAPDH was used as an internal control. Data are presented as mean values±SEM. The p-value was determined by a two-tailed, unpaired Mann-Whitney test, n=3 for Lenti-Vec Con, and n=3 for ALDH1A1 o/e.

FIG. 11S: Immunoblot analyses of lysates from H1650-Tr-BrM-S100A9i cells expressing either Lenti-Vec Con or ALDH1A1 o/e using antibodies against ALDH1A1 and β-actin (loading control). Data is representative of three independent experiments.

FIG. 11T: Ex-vivo photon flux of brains from mice injected with H1650-Tr-BrM-S100A9i cells expressing either Lenti-Vec Con or ALDH1A1 o/e was determined by bioluminescence imaging. Mice were collected at 7 weeks post tumor-cell injection. The photon flux scale is indicated on the right side of the image.

FIG. 11U: Violin plots depicting normalized photon flux of brains imaged ex vivo from the mice represented in (t). The normalized photon flux for brain was calculated by dividing the photon flux from brain collected ex-vivo by the total photon flux at day 0 (i.e., the day of injection) and multiplying that value by 100. Data are presented as mean values±SEM. P-values were determined by a two-tailed, unpaired Mann-Whitney test: n=6 for Lenti-Vec-Con, and n=6 for ALDH1A1o/e.

FIG. 11V: Representative images of CK7 immunohistochemistry in brain sections from mice injected with either H1650-Tr-BrM-S100A9i-Lenti-Vec Con cells (upper panel) or H1650-Tr-BrM-S100A9i-ALDH1A1 o/e cells (lower panel). Mice were euthanized at 7 weeks post tumor-cell injection. Scale bars, 200 μm.

FIG. 11W: Quantitative analysis of the percentage of CK7-immunostained brain sections covered by metastasis that are represented in panel (v). Data are presented as mean values±SEM. P-values were determined by a two-tailed, unpaired Mann-Whitney test: n=5 for Lenti-Vec-Con, and n=5 for ALDH1A1 o/e.

FIG. 11X: In-vitro cell proliferation assay of PC9-Tr-BrM cells expressing either Lenti-Con or ALDH1A1i guide-RNAs (left panel) and H1650-Tr-BrM cells expressing either Lenti-Con or ALDH1A1i guide-RNAs (right panel). Data are presented as the mean cell number±SEM and are representative of three independent experiments.

FIG. 11Y: In-vitro cell proliferation assay of PC9-Tr-BrM-S100A9i cells expressing either Lenti-Vec Con or ALDH1A1 o/e (left panel) and H1650-Tr-BrM-S100A9i cells expressing either Lenti-Vec Con or ALDH1A1 o/e (right panel). Data are presented as the mean cell number±SEM and are representative of three independent experiments.

### Site-Specific Effects of the S100A9-ALDH1A1 Axis.

FIG. 12A-FIG. 12D: Immunostaining analysis of S100A9 (12a and c) or ALDH1A1 (12b and d) on lung (12a and b) and brain (12c and d) tissue sections from mice injected with H1650-BrM cells and treated with either vehicle (“Veh”, top panels) or upon relapse post osimertinib treatment (“Osi-relapse”, bottom panels). Cells were injected into the arterial circulation of immunodeficient mice via intracardiac injection. After metastatic signal was detected by bioluminescence imaging, treatment was administered at 25 days post tumor-cell injection with either vehicle or osimertinib at 5 mg/kg body weight/day by oral gavage, 5 days per week. Brain and lung tissues were collected at endpoint (2 months for the vehicle group and 4 months post tumor-cell injection for the Osi-relapse group). Scale bars, 50 μm. Data are representative of 3 independent experiments.

FIG. 12E-FIG. 12F: Quantitative analysis of the S100A9- and ALDH1A1-positive cells stained in 12(a-d). Immunostained sections were counted using the Qu-Path software where positively stained cells were identified by setting a threshold for signal intensity (1+). Data are presented as mean values±SEM. The p-value was determined by a two-tailed, unpaired Mann-Whitney test. For S100A9-positive lung lesions: n=6 for Veh, n=9 for Osi-relapse. For ALDH1A1-positive lung lesions: n=14 for Veh, n=10 for Osi-relapse. For S100A9-positive brain lesions: n=5 for Veh, n=6 for Osi-relapse. For ALDH1A1-positive brain lesions: n=15 for Veh, n=6 for Osi-relapse.

FIG. 12G: Schematic representation of the experimental protocol for injecting H1650-Tr-BrM cells stably expressing either Lenti-Con, S100A9i, or ALDH1A1i guide-RNAs, or S100A9i guide-RNA plus ALDH1A1 o/e, via intracardiac injection. Mice were euthanized at 7 weeks post tumor-cell injection.

FIG. 12H: Ex-vivo photon flux of lungs from mice injected with H1650-Tr-BrM cells expressing either Lenti-Con, S100A9i, ALDH1A1i, or S100A9i plus ALDH1A1 o/e was determined by bioluminescence imaging. Lungs were collected from mice of the indicated groups at 7 weeks post tumor-cell injection. The photon flux scale is indicated on the right side of the image.

FIG. 12I: Violin plots depicting normalized photon flux of lungs imaged ex-vivo from the mice described in panel 12(h). The normalized photon flux for lung was calculated by dividing the photon flux from lung collected ex-vivo by the total photon flux at day 0 (i.e., the day of injection) and multiplying that value by 100. Data are presented as mean values±SEM. P-values were determined by a two-tailed, unpaired Mann-Whitney test. Left graph: n=12 for Lenti-Con, n=10 for S100A9i. Middle graph: n=14 for Lenti-Con, n=10 for ALDH1A1i. Right graph: n=10 for S100A9i/Lenti-Vec Con, n=9 for S100A9i/ALDH1A1 o/e.

FIG. 12J: Expression of S100A9 and ALDH1A1 was determined by qRT-PCR analysis in H1650-lung-derivatives expressing either Lenti-Con or S100A9i guide-RNAs (left most panel), either Lenti-Con or ALDH1A1i guide-RNAs (middle panel), and either S100A9i/Lenti-Vec Con or S100A9i/ALDH1A1 o/e (right panel). GAPDH was used as an internal control. Data are presented as mean values±SEM. The p-value was determined by a two tailed, unpaired Mann-Whitney test: n=4 per group for all panels.

FIG. 12K: Schematic representation of the experimental protocol for lung orthotopic injections. H1650-Tr-BrM-lung derivatives stably expressing either Lenti-Con, S100A9i, ALDH1A1i, or S100A9i plus ALDH1A1 o/e were injected in the lung (intrathoracic injections, labeled as “lung orthotopic injection” in the figure). Mice were euthanized at 21 days post tumor-cell injection for analysis.

FIG. 12L: Violin plot depicting normalized photon flux of lungs imaged ex-vivo from the experiment described in panel 12(k). The normalized photon flux for lung was calculated by dividing the photon flux from lung collected ex-vivo by the total photon flux at day 0 (i.e., the day of injection) and multiplying that value by 100. Data are presented as mean values±SEM. P-values were determined by a two-tailed, unpaired Mann-Whitney test: n=8 for Lenti-Con, n=12 for S100A9i, n=11 for ALDH1A1i and n=7 for S100A9i plus ALDH1A1 o/e.

FIG. 12M-FIG. 12P: Representative images of bone and brain sections from vehicle- and osimertinib-treated mice that were stained with S100A9 (12(m-n)) or ALDH1A1 antibodies (12(o-p)). PC9-BrM cells were injected into the arterial circulation of immunodeficient mice via intracardiac injection. After metastatic signal was detected by bioluminescence imaging, treatment was administered at 25 days post tumor-cell injection with either vehicle or osimertinib at 5 mg/kg body weight/day by oral gavage, 5 days per week. Brain and lung tissues were collected at endpoint (2 months in the “Veh” group and 5 months post tumor-cell injection in the osimertinib-residual-tumor group (abbreviated as “Osi-Res. tumor”). The images are representative of 10 mice analyzed per group. Scale bars, 50 μm (12(m-o)) and 100 μm (12(p)).

FIG. 12Q-FIG. 12R: Immunostained images described in panels 12(m-p) were quantitated using automated Qu-Path software to count S100A9- or ALDH1A1-positive cancer cells that were identified by setting a threshold for signal intensity (1+). Data are presented as mean values±SEM. P-values (indicated in the figures) were determined by a two-tailed, unpaired Mann-Whitney test. For S100A9 (see 12(q)), n=9 for PC9/Bone/Veh metastases, n=13 for PC9/Bone/Osi-Res. tumor metastases. n=4 for PC9/Brain/Veh metastases, and n=9 for PC9/Brain/Osi-Res. tumor metastases. For ALDH1A1 (see 12(r)), n=7 for PC9/Bone/Veh metastases, n=8 for PC9/Bone/Osi-Res. tumor metastases, n=5 for PC9/Brain/Veh metastases, and n=5 for PC9/Brain/Osi-Res. tumor metastases.

FIG. 12S: Expression of S100A9 and ALDH1A1 was determined by qRT-PCR analysis in PC9-bone-derivative cells stably expressing either Lenti-Con, S100A9i, or ALDH1A1i guide-RNAs, or S100A9i plus either Lenti-Vec Con or ALDH1A1 o/e. GAPDH was used as an internal control. Data are represented as mean values±SEM. The p-value was determined by a two-tailed, unpaired Mann-Whitney test. Left graph: n=3 for Lenti-Con, n=6 for S100A9i. Middle graph: n=4 for Lenti-Con, n=4 for ALDH1A1i. Right graph: n=4 for S100A9i plus Lenti-Vec Con, n=4 for S100A9i plus ALDH1A1 o/e.

FIG. 12T: Schematic representation of the experimental protocol for intratibial injections of PC9-Tr-BrM-bone derivative cells stably expressing either Lenti-Con, S100A9i, or ALDH1A1i guide-RNAs, or S100A9i plus ALDH1A1 o/e. Mice were euthanized at 21 days post tumor-cell injection.

FIG. 12U: Violin plots depicting normalized photon flux of bone imaged from the mice described in panel (t). The normalized photon flux for bone was calculated by dividing the photon flux from bone collected ex-vivo by the total photon flux at day 0 (i.e., the day of injection) and multiplying that value by 100. Data are presented as mean values±SEM. P-values were determined by a two-tailed, unpaired Mann-Whitney test. Left graph: n=5 for Lenti-Con, n=4 for S100A9i. Middle graph: n=5 for Lenti-Con, n=4 for ALDH1A1i. Right graph: n=4 for S100A9i plus Lenti-Vec Con, n=4 for S100A9i plus ALDH1A1 o/e.

### Sensitivity of Osimertinib-Refractory Brain Metastases to RAR Inhibition.

FIG. 13A-FIG. 13B: STRA6 expression was determined by qRT-PCR analysis of Tr-BrM cells from the PC9 model (13(a)) and H1650 model (13(b)), which were treated with either DMSO, retinol or retinol plus different retinoic acid receptor antagonists. Cells were cultured for two days in serum-free RPMI medium with DMSO alone, with 5 μM retinol alone, or medium with both 5 μM retinol and 5 μM antagonists (either the RAR-α antagonist BMS-195614, the RAR-γ antagonist MM-11253, or the pan-RAR antagonist AGN-194310). After two days of culturing, cells were washed and collected for RNA analysis. GAPDH was measured as an internal control. Data are presented as mean values±SEM. P-values were determined by the Kruskal-Wallis test. For the PC9 model in 13(a), n=8 for vehicle; n=5 for retinol; n=3 for retinol plus the RAR-alpha inhibitor (“RAR-α-inh.”); n=3 for retinol plus the RAR-gamma inhibitor (“RAR-γ-inh.”); n=4 for retinol plus the pan-RAR inhibitor (“pan-RAR-inh.”). For the H1650 model in 13(b), n=7 for all groups.

FIG. 13C: Cell viability assay for comparing in-vitro sensitivity of the indicated cell lines to pan-RAR-inhibition (AGN194310, abbreviated as “AGN”). Cell viability values are normalized to the vehicle (DMSO)-treated control, presented as the mean values±SEM, and representative of three independent experiments. P-values were determined by two-way ANOVA with Tukey's test.

FIG. 13D-FIG. 13E: S100A9 (13(d)) and ALDH1A1 (13(e)) expression was determined by qRT-PCR analysis in the indicated cell lines. PC9-Tr-BrM cells were used as a positive control, and all cell lines were normalized to PC9-BrM. GAPDH was used as an internal control. Data are presented as mean values±SEM. Data are representative of three independent experiments. The p-value was determined by a two-tailed, unpaired Mann-Whitney test. For (d), n=9, 5, 6 and 6 for PC9-BrM, PC9-Tr-BrM, H1975 and HCC4006 respectively. For (e), n=6, 5, 3 and 3 for PC9-BrM, PC9-Tr-BrM, H1975 and HCC4006, respectively.

FIG. 13F-FIG. 13G: Quantitation of photon flux (normalized to the initial day of injection) of brain by longitudinal imaging in mice injected with either PC9-Tr-BrM (13(f)) or H1650-Tr-BrM (13(g)) via intracardiac injections. At 25 days post tumor-cell injection after confirmation of metastatic signal, mice were administered long-term treatment with either 1) vehicle (“Veh”), 2) osimertinib (“Osi”, 5 mg/kg body weight/day), 3) AGN194310 (“Pan-RAR-inh”, 0.5 mg/kg body weight/day), or 4) osimertinib (5 mg/kg body weight/day) plus AGN194310 (0.5 mg/kg body weight/day) by oral gavage, 5 days per week, until the endpoint (7 weeks post tumor-cell injection). For panel 13(f): n=5 for Veh, n=5 for Osi, n=7 for Pan-RAR-inh, and n=5 for Osi plus Pan-RAR-inh. For panel 13(g), n=5 for Veh, n=4 for Osi, n=7 for Pan-RAR-inh, and n=8 for Osi plus Pan-RAR-inh. P-values were determined by the Kruskal-Wallis test for both (f) and (g).

FIG. 13H-FIG. 13I: Immunoblot analysis of RARα and RARγ expression in PC9-Tr-BrM (13(h)) and H1650-Tr-BrM (13(i)) cells stably transduced with shRNA set #1 expressing either control shRNA (“sh-Con”) or shRARα plus shRARγ (“sh-RAR”) using antibodies against RARα, RARγ and β-actin (loading control). The immunoblot image is representative of three independent experiments.

FIG. 13J-FIG. 13K: Immunoblot analysis of RARα and RARγ expression in PC9-Tr-BrM (13(j)) and h1650-Tr-BrM (13(k)) cells stably transduced with shRNA set #2 expressing either control shRNA (“sh-Con”) or shRARα plus shRARγ (“sh-RAR”) using antibodies against RARα, RARγ and β-actin (loading control). The immunoblot image is representative of three independent experiments.

FIG. 13L-FIG. 13M: Violin plots depicting normalized photon flux of brain imaged from the mice injected by intracardiac injection with Tr-BrM from the PC9 model (13(l)) or the H1650 model (13(m)) that were stably transduced with shRNA set #2 expressing either control shRNA (“sh-Con”) or shRARα plus shRARγ (“sh-RAR”). Mice were euthanized at 7 weeks post tumor-cell injection. The normalized photon flux for brain was calculated by dividing the photon flux from brains collected ex-vivo by the total photon flux at day 0 (i.e., the day of injection) and multiplying that value by 100. Data are presented as mean values±SEM. P-values were determined by a two-tailed, unpaired Mann-Whitney test: n=5 per group for all groups.

FIG. 13N: Representative images of CK7 immunohistochemistry in brain sections from mice injected with either PC9-Tr-BrM sh-Con (left-upper panel), PC9-Tr-BrM sh-RAR (Set 2, left-lower panel), H1650-Tr-BrM sh-Con (right-upper panel) or H1650-Tr-BrM sh-RAR (Set 2, right-lower panel) described in panels (l-m). Scale bars: 500 μm for the PC9 model and 100 μm for the H1650 model.

FIG. 13O: Normalized photon flux of brain from bioluminescence imaging from mice injected with PC9- or H1650-derived Tr-BrM cells stably transduced with shRNA set #2 containing either control shRNA (sh-Con) or shRARα plus shRARγ (shRAR) described in panels 13(l-n). The normalized photon flux for brain was calculated by dividing the photon flux from brains collected ex-vivo by the total photon flux at day 0 (i.e., the day of injection) and multiplying that value by 100. Data are presented as mean values±SEM. P-values were determined by a two-tailed, unpaired Mann-Whitney test. For the PC9 model: n=7 for sh-Con, and n=10 for sh-RAR. For the H1650 model, n=8 for sh-Con and n=4 for sh-RAR.

FIG. 13P-FIG. 13Q: Violin plots depicting normalized photon flux of lung measured by bioluminescence imaging of mice. Mice were injected with either PC9- or H1650-derivatives (13(p) and 13(q), respectively) directly into the lung (intrathoracic injections as depicted in FIG. 12k and treated with either vehicle or pan-RAR inhibitor AGN194310 (“Pan-RARi”) for 2 weeks. The normalized photon flux for lung was calculated by dividing the photon flux from lung collected at endpoint (3 weeks post tumor-cell injection) ex vivo by the total photon flux at day 0 (i.e., the day of injection) and multiplying that value by 100. Data are presented as mean values±SEM. P-values were determined by a two-tailed, unpaired Mann-Whitney test. For panel (p): n=8 for Veh, and n=11 for Pan-RARi. For panel (q): n=7 for Veh, and n=8 for Pan-RARi.

FIG. 13R-FIG. 13S: Violin plots depicting normalized photon flux of bone measured by bioluminescence imaging of mice. Mice were injected with either PC9- or H1650-derivatives (13(r) and 13(s), respectively) directly into the tibia bone as depicted in FIG. 12t and treated with either vehicle or AGN194310 (“Pan-RARi”) for 2 weeks. The normalized photon flux for bone was calculated by dividing the photon flux from bone collected at endpoint (3 weeks post tumor-cell injection) ex vivo by the total photon flux at day 0 (i.e., the day of injection) and multiplying that value by 100. Data are presented as mean values±SEM. P-values were determined by a two-tailed, unpaired Mann-Whitney test. For panel (r), n=6 for Veh, and n=4 for Pan-RARi. For panel(s), n=6 for Veh, and n=5 for Pan-RARi.

### DETAILED DESCRIPTION

### Cancer Progression and Lethal Brain Relapse in Osimertinib-Treated EGFR-Mutant Metastatic Lung Cancer Models.

To model osimertinib response and relapse in mice, we used the human EGFR-mutant, PC9-BrM3 lung cancer metastasis model (30,31), which metastasizes to the brain, bone and lymph nodes (31). The PC9-BrM3 cell line (referred to as “PC9-BrM” hereafter) was derived by in-vivo selection for PC9 lung cancer cells (containing an EGFR exon-19 deletion) with a high incidence of brain metastasis. We engineered PC9-BrM cells to express luciferase for monitoring metastasis development by bioluminescence imaging and injected them into the arterial circulation of immunodeficient mice via intracardiac injection (FIG. 1a).

After confirmation of metastatic signal at 25 days post-injection by bioluminescence imaging, we initiated a long-term treatment study involving the regular administration of either vehicle or osimertinib (5 mg/kg body weigh/day), five days per week. We monitored metastasis weekly by bioluminescence imaging (FIG. 1b).

As in human patients (15,16,32,33), osimertinib significantly prolonged brain-metastasis-progression-free survival, from 47 days to 144 days (p<0.0001), compared to vehicle-treated mice; however, all drug-treated mice eventually developed brain relapse and died (FIG. 1b-c). Interestingly, while cancer cells in the extracranial sites (bone and lymph nodes) in the body did not progress during continuous osimertinib treatment (FIG. 1b and FIG. 8a), brain metastases gradually progressed (FIG. 1b-c and FIG. 8a).

To understand the underlying mechanisms of brain relapse using this model, we isolated brain metastatic cells from the relapsed brain of osimertinib-treated mice and designated them PC9-Tr-BrM (Treated Brain Metastatic, FIG. 1d). We then injected either PC9-BrM or PC9-Tr-BrM cells into the arterial circulation of naive immunodeficient mice and treated them with either vehicle or osimertinib.

We found that the brain-metastasis-progression-free survival of mice injected with PC9-Tr-BrM cells was no longer increased by osimertinib treatment (FIG. 1e). Instead, accelerated progression in the brain was observed in the PC9-Tr-BrM-injected mice compared to PC9-BrM-injected mice, as determined by histological analysis of brain metastasis surface area with cytokeratin-7 (CK7) immunostaining, despite continued osimertinib treatment (FIG. 1f-g).

To validate these observations using a second, independent, EGFR-mutant lung cancer model, we engineered H1650 lung cancer cells (harboring EGFR exon-19 and PTEN deletions (34)) to express luciferase and injected them into immunodeficient mice to derive a new brain metastatic cell line (designated H1650-BrM) by the in-vivo selection method (35), FIG. 8b-c. We injected H1650-BrM cells into the arterial circulation of immunodeficient mice via intracardiac injection (FIG. 8d), and after confirmation of metastasis development at 25 days post-injection, we initiated a long-term treatment study involving the regular administration of either vehicle or osimertinib (5 mg/kg body weigh/day), five days per week.

Consistent with the PC9-derived model, osimertinib prolonged brain-metastasis-progression-free survival in the H1650-BrM model (FIG. 8e), albeit for a shorter duration than the PC9-BrM model (FIG. 1c). After a striking response period of 120 days, 100% of the osimertinib-treated mice developed brain relapse (45% of which also developed lung lesions) and died (FIG. 8e).

We then isolated brain metastatic cells from the osimertinib-treated mice (designated H1650-Tr-BrM), injected them into the arterial circulation of naive immunodeficient mice and treated the mice with either vehicle or osimertinib. Analogous to the PC9-Tr-BrM model (FIG. 1e), the brain-metastasis-progression-free survival of mice injected with H1650-Tr-BrM cells was no longer prolonged by osimertinib treatment (FIG. 8f), with rapid progression to the brain in 100% of the mice (FIGS. 8f-h).

These results show that osimertinib initially delays metastatic progression, but eventually drug-tolerant cancer cells escape treatment and cause lethal brain relapse in two independent, EGFR-mutant, metastatic lung cancer models.

### S100A9 is a Key Mediator of Brain Relapse in Osimertinib-Refractory Lung Cancer Cells.

To investigate the mechanisms of brain relapse from osimertinib treatment, we first explored whether Tr-BrM cells still showed EGFR pathway inhibition in response to osimertinib. We found that osimertinib treatment led to a similar dose-dependent inhibition of EGFR and ERK phosphorylation and similar cytotoxicity profiles in the BrM and Tr-BrM derivatives from both PC9 and H1650 cell lines (FIG. 2a and FIG. 9a-c), thus confirming effective target inhibition of the EGFR pathway and cytotoxicity in vitro by osimertinib.

To determine whether osimertinib effectively inhibits EGFR pathway activation in situ in the brain, we next performed immunostaining analysis of phospho-EGFR tyrosine 1068 (p-EGFR) on brain sections from mice injected with PC9-BrM and H1650-BrM cells and treated with either vehicle or osimertinib. Consistent with our in-vitro findings (FIG. 2a and FIG. 9a), we found that p-EGFR was significantly reduced in both micro- and relapsed-metastatic lesions in osimertinib-treated mice compared to the vehicle-treated control (FIGS. 2b-c and FIGS. 9d-e). However, in contrast to the in-vitro findings, drug-tolerant cells were able to thrive in the brain by EGFR pathway-independent mechanisms. These results suggest that brain metastatic cells are able to resist the anti-proliferative and cytotoxic effects of osimertinib-mediated EGFR inhibition and grow in the brain.

To identify pathways that promote the growth and survival of Tr-BrM cells in the brain, we performed quantitative label-free mass spectrometry and transcriptomics comparing PC9-BrM and PC9-Tr-BrM cells (FIG. 2d). S100A8 and S100A9, two calcium-binding proteins that form a heterodimer and are normally secreted by myeloid cells (36,37), emerged as the top upregulated candidates in the PC9-Tr-BrM cells compared to PC9-BrM cells by proteomic profiling (FIG. 2e). Functional annotation analysis using the Database for Annotation, Visualization, and Integrated Discovery (DAVID) showed S100A8 and S100A9 were enriched in 23 out of 30 (77%) pathways (See Biswas, Cancer Discovery, at Supplementary Table 1-2).

Transcriptomic profiling by RNA sequencing (RNA-seq) and functional enrichment analysis by G: Profiler (38) identified broad gene ontology (GO) categories that were significantly enriched in the PC9- and H1650-derived BrM and Tr-BrM cells (FIG. 9f-I). Consistent with the proteomics analysis (FIG. 2e), S100A8 and S100A9 were also among the significantly upregulated genes by RNA-seq in the Tr-BrM cells compared to BrM cells from both PC9 and H1650 models (FIG. 2f, FIG. 9f-g) and Biswas, Cancer Discovery, at Supplementary Table 3-4). Increased expression of S100A8 and S100A9 was validated by immunoblot and quantitative RT-PCR analyses in both PC9- and H1650-derived Tr-BrM cells, compared to their respective BrM controls (FIG. 2g-h and FIG. 10a-b).

Immunostaining analysis of brain sections showed prominent intracellular expression of S100A9 in metastatic cells derived from both PC9 and H1650 models (FIG. 10c-d). Consistent with these observations, secreted S100A9 was not detected in the sera from mice bearing brain metastases in either the PC9- or H1650-derived models by ELISA analysis, and the majority of S100A9 was detected in the cell lysate rather than the culture supernatant (see Biswas, Cancer Discovery, at Supplementary Table 5).

To determine whether S100A8/S100A9 expression is causally linked with brain metastasis development, we performed loss-of-function studies. We suppressed the expression of S100A8 and S100A9 using CRISPR-repression (CRISPRi) in both PC9- and H1650-derived Tr-BrM cells (FIG. 2i, FIG. 10e-I). We found that individual repression of S100A8 and S100A9 in Tr-BrM cells derived from both PC9 and H1650 cell lines using independent guide RNAs (gRNAs) led to a significant reduction in brain metastasis at endpoint (7 weeks post-injection) as determined by quantitative bioluminescence imaging and by histological analysis of metastasis surface area with CK7 immunostaining (FIG. 2j-m and FIG. 10j-o). S100A8 and S100A9 function together as a heterodimer, and S100A8 expression is downregulated in S100A9-deficient neutrophils (28,36).

We therefore examined whether S100A9 repression (S100A9i) leads to downregulation of S100A8 in EGFR-mutant lung cancer cell lines. Indeed, S100A8 expression was significantly reduced in the PC9- and H1650-derived Tr-BrM-S100A9i cells compared to their respective lenti-control Tr-BrM cells (FIG. 10p-q). We used S100A9 repression as a surrogate for studying the functional loss of S100A8/9 in Tr-BrM cells in subsequent experiments. Our results demonstrate that elevated expression of S100A9 (and S100A8) promotes brain metastasis of EGFR-mutant lung cancer cells and becomes an alternative mechanism to thrive in the brain while under stress from EGFR pathway inhibition.

### S100A9-Proficient Cancer Cells Promote Post-Colonization Growth in the Brain.

The ability of cancer cells to develop metastases in the brain depends on their ability to extravasate from blood vessels into the brain parenchyma (known as metastatic seeding) and subsequently adapt, survive, and grow in the brain microenvironment (known as post-colonization outgrowth, (39-41)). To determine how S100A9 drives brain metastasis, we asked which of these steps during brain metastasis require S100A9 expression.

To test whether S100A9 is required for metastatic seeding in the brain, we injected PC9-derived Tr-BrM cells expressing lenti-control (Tr-BrM-Lenti-con) or S100A9i (Tr-BrM-S100A9i) into the arterial circulation of immunodeficient mice via intracardiac injection (FIG. 3a). Seven days following injection, a timepoint when lung cancer cells extravasate and can be detected in the brain parenchyma (41), we harvested, sectioned and immunostained brain tissues with an antibody against CK7 to quantitate the number of cancer cells that seeded in the brain. We found no difference in the number of extravasated cells in the brain parenchyma between the S100A9-proficient and -deficient groups (FIG. 3b), indicating that S100A9 is not required for metastatic seeding in the brain in the PC9-derived model.

To test whether S100A9 is instead required for post-colonization growth in the brain, we immunostained brain sections from mice harvested 7 weeks following tumor-cell injection (FIG. 3c), using an antibody against phospho-histone H3 (Ser-10) (FIG. 3d). Consistent with larger metastatic lesions in the Tr-BrM-Lenti-con group compared to the Tr-BrM-S100A9i group (FIG. 2I-m), a significantly higher number of mitotically active, phospho-histone-H3-positive cells was observed in the Tr-BrM-Lenti-con group (FIG. 3d-e). However, no differences in proliferation were observed in vitro between the BrM and Tr-BrM cells, or the Tr-BrM-Lenti-con and Tr-BrM-S100A9i cells, that were derived from either PC9 or H1650 cell lines (FIGS. 10r-u). These results indicate that S100A9 is required for post-colonization growth of metastatic lung cancer cells in situ in the brain.

Based on these results, we reasoned that although both S100A9-proficient and -deficient cells can colonize the brain, the S100A9-proficient cells are likely to grow better in the brain, a biological trait that can potentially sustain their growth and survival after EGFR pathway inhibition by osimertinib. In line with this hypothesis, we observed the presence of both S100A9^{high} and S100A9^{low} cells in brain sections from PC9-BrM-injected mice treated with vehicle for two months (FIG. 3f, Vehicle).

However, this scenario changed dramatically after prolonged osimertinib treatment where S100A9^{high} cells became prominent in the surviving metastatic cells following 3 months of osimertinib treatment (FIG. 3f, MRD), and predominated in relapsed brain metastases following 8 months of osimertinib treatment (FIG. 3f, Relapse). Based on these findings, we reasoned that if osimertinib treatment selects for S100A9^{high} cells, then the PC9-BrM cell line should exhibit heterogeneity with respect to pre-existing S100A9 expression levels that are present prior to drug treatment and brain metastasis. Indeed, single-cell cloning of the PC9-BrM line gave rise to distinct S100A9^{high} and S100A9^{low} single-cell-derived progenies (SCPs) in culture (FIG. 3g-h), which were then compared for their ability to grow in the brain.

Bioluminescence imaging showed a striking increase in brain metastasis by S100A9^{high} SCPs compared to S100A9^{low} SCPs when an equal number of cells from each group were injected into the arterial circulation of immunodeficient mice (FIG. 3i-k). These results were further validated by histological analysis of metastasis surface area with CK7 immunostaining (FIG. 3I-m).

These findings indicate that osimertinib treatment selects S100A9^{high} cells for growth and survival in the brain, from a pre-existing pool of lung cancer cells that exhibit heterogeneity for S100A9 expression. S100A9^{high} cells thereby serve as seeds of future relapse from osimertinib treatment.

Association of S100A9 Expression with Brain Metastasis and Shorter PFS in Osimertinib-Treated Lung Cancer Patients.

Our preclinical studies revealed two distinct functions of S100A9: to promote brain metastatic growth, and to escape the growth-inhibitory effects of osimertinib. To clinically validate our experimental findings, we performed S100A9 immunostaining on tissue specimens that were obtained prior to osimertinib treatment from 29 EGFR-mutant lung cancer patients (FIG. 4a and see Biswas, Cancer Discovery, at Supplementary Table 6).

The immunostained samples were scored as either S100A9-positive (any percentage of clear, positive intracellular S100A9 staining in cancer cells) or S100A9-negative (no detectable S100A9 staining in cancer cells, FIG. 4a). Consistent with our preclinical observations (FIG. 2j-m and FIG. 3j-m), an independent blinded pathological examination revealed a statistically significant association between S100A9 expression and the development of brain metastasis (p=0.0027, FIG. 4b).

We next asked whether S100A9 expression in pre-osimertinib-treatment cancer cells correlated with osimertinib treatment response in a combined cohort of patients on first-, second- and third-line osimertinib treatment (FIG. 4c-d). Indeed, high expression of S100A9 in cancer cells from pre-osimertinib-treatment samples correlated significantly with worse PFS on osimertinib (n=29, p=0.0011) both in the combined cohort (FIG. 4d) and when stratified by treatment lines (n=17, p=0.0106 for first-line osimertinib patients, and n=12, p=0.0451 for second- and third-line patients, FIG. 4e). Therefore, based on our preclinical studies and clinical validation, elevated S100A9 expression in cancer cells is significantly associated with brain metastasis and strongly correlates with progression in osimertinib-treated lung cancer patients.

### S100A9 Promotes Brain Relapse Through ALDH1A1.

To further explore how S100A9 mediates the growth of brain metastatic lesions, we analyzed the transcriptome of S100A9-proficient (Tr-BrM-Lenti-con) and S100A9-deficient (Tr-BrM-S100A9i) brain metastatic cells from the PC9- and H1650-derived models by RNA-seq (FIG. 5a-b, FIG. 11a-c, and see Biswas, Cancer Discovery, at Supplementary Table 7 and 8). Consistent with our previous results (FIG. 10p-q), S100A8 was among the top downregulated genes in both PC9- and H1650-derived Tr-BrM-S100A9i cells (FIG. 5a-b, FIG. 11a, and see Biswas, Cancer Discovery, at Supplementary Table 7-8).

Interestingly, aldehyde dehydrogenase 1 family 1A1 (ALDH1A1), which encodes an enzyme that catalyzes the conversion of retinaldehyde to retinoic acid, was among the top downregulated genes in both PC9- and H1650-derived-Tr-BrM-S100A9i cells (FIG. 5b and FIG. 11a). Gene set enrichment analysis (GSEA) further revealed a significant decrease in the expression of retinal metabolism genes in the Tr-BrM-S100A9i cells (GOBP Retinoic Acid Metabolic Process, p=0.024 and KEGG Retinal Metabolism, p=0.026, FIG. 11d-e. Moreover, the analysis of genes present at the leading edges of both the GOBP Retinoic Acid Metabolic Process and KEGG Retinal Metabolism gene sets confirmed enrichment for genes associated with retinoic acid metabolism in PC9-Tr-BrM compared to PC9-BrM cells (FIG. 11f). The leading-edge genes from both GOBP Retinoic Acid Metabolic Process and KEGG Retinal Metabolism were also significantly downregulated upon S100A9 repression in PC9-Tr-BrM cells (FIG. 11g). These results suggest that S100A9 activates the retinoic acid pathway in Tr-BrM cells.

Retinoic acid (RA), an active metabolite of retinal (vitamin A), binds to nuclear hormone receptors to regulate diverse cellular processes, including proliferation, tissue remodeling and differentiation (42,43). For RA biosynthesis, retinal (vitamin A) is first oxidized by alcohol dehydrogenase (ADH) enzymes to retinaldehyde. Retinaldehyde is further oxidized to RA by the aldehyde dehydrogenase (ALDH) family of enzymes, mainly ALDH1A1, ALDH1A2 and ALDH1A3.

Among the ALDH1A family members, ALDH1A1 was only significantly downregulated by S100A9 repression in both PC9- and H1650-derived Tr-BrM cells (FIG. 11h). We confirmed a reduction in RNA and protein expression of ALDH1A1 in PC9- and H1650-derived Tr-BrM-S100A9i cells compared to their respective controls (FIG. 5c-d, FIG. 11i-j). Moreover, immunohistochemical analysis showed robust expression of ALDH1A1 and a striking overlap between S100A9 and ALDH1A1 in brain metastatic lesions from the PC9-Tr-BrM model (FIG. 5e).

Importantly, ALDH1A1 was significantly downregulated by S100A9 repression in brain metastatic lesions from the PC9- and H1650-Tr-BrM models (Fig. Sf-g and FIG. 11k). We therefore asked whether S100A9 promotes brain metastasis through upregulation of ALDH1A1.

To test this possibility, we first analyzed whether repression of ALDH1A1 can phenocopy S100A9i in PC9- and H1650-derived Tr-BrM cells. We confirmed successful repression of ALDH1A1 in PC9- and H1650-derived Tr-BrM cells (FIG. 5h and FIG. 11l), and found that ALDH1A1 repression in the Tr-BrM cells significantly reduced brain metastasis as quantified by bioluminescence imaging and by histological analysis of metastasis surface area with CK7 immunostaining (FIG. 5i-I and FIG. 11m-p). We also found that forced expression of ALDH1A1 (“ALDH1A1o/e”) was sufficient to rescue the S100A9i phenotype in both PC9- and H1650-derived models (FIG. 5m-q and FIG. 11q-w), indicating that ALDH1A1 represents a key downstream effector of S100A9 that mediates brain metastasis.

No differences in proliferation were observed in vitro among the PC9- and H1650-Tr-BrM cells transduced with lentivirus encoding either Lenti-con, S100A9i, ALDH1A1i or S100A9i-ALDH1A1o/e, suggesting that the S100A9-ALDH1A1-RA axis is not required for cell growth in vitro (FIGS. 11x-y). These results therefore demonstrate that osimertinib-refractory lung cancer cells co-opt the S100A9-ALDH1A1 signaling axis to survive and grow in the brain despite inhibition of EGFR activity by osimertinib.

### Site-Specific Effects of the S100A9-ALDH1A1 Axis

We next asked whether the S100A9-ALDH1A1 axis promotes the growth of cancer cells selectively in the brain or if it also promotes growth in the lung and bone, two additional sites of growth for H1650- and PC9-derivatives, respectively (FIG. 1 and FIG. 8). Since H1650-derived BrM cells can grow in both the brain and lung (FIGS. 8c and 8e), we first asked whether the expression of S100A9 and ALDH1A1 is elevated in H1650-BrM-derived lung lesions post osimertinib treatment (referred to as “Osi-relapse”) compared to lung lesions from vehicle-treated mice, similar to what we observe for brain lesions (FIGS. 2-3, FIG. 10).

To address this question, we first injected H1650-BrM cells into the arterial circulation of immunodeficient mice, treated them with either vehicle or osimertinib, and then harvested lung and brain tissues for immunohistochemical analysis. Lung and brain tissues were collected at endpoint (2 months in the vehicle-treated group and 4 months post tumor-cell injection in the Osi-relapse group).

In contrast to the brain lesions, lung lesions showed no statistically significant increase in S100A9 and ALDH1A1 expression from the Osi-relapse group compared to the vehicle-treated group (FIG. 12a-f). To evaluate the requirement of the S100A9-ALDH1A1 axis for cancer cell growth in the lung, we injected H1650-Tr-BrM cells (expressing high S100A9 and ALDH1A1 levels) that were transduced with lentivirus encoding either Lenti-con, S100A9i (FIG. 2i), ALDH1A1i (FIG. 11l), or S100A9i-ALDH1A1o/e (FIG. 11r)) into the arterial circulation of immunodeficient mice via intracardiac injection (FIG. 12g). Compared to the robust brain metastasis phenotype (FIG. 2j-m, FIG. 11m-p, FIG. 11t-w), we observed a modest but statistically significant reduction in the growth of lung lesions upon suppression of S100A9 or ALDH1A1, and a rescue by ALDH1A1 expression (in a S100A9i background), as determined by quantitative bioluminescence imaging (FIG. 12h-I).

To confirm these findings, we isolated H1650 lung derivatives from osimertinib-treated mice injected with H1650-BrM cells (abbreviated as “H1650-lung derivatives”) and transduced them with either Lenti-con, S100A9i, ALDH1A1i or S100A9i-ALDH1A1o/e (FIG. 12i). We then directly implanted these cells in the lung of immunodeficient mice and evaluated the growth of these cells in the lung at endpoint (3 weeks post-injection, FIG. 12k). Consistent with our previous findings (FIG. 12h-i), a modest but statistically significant reduction was observed in lung tumor growth upon S100A9 or ALDH1A1 repression, which was rescued by forced ALDH1A1 expression (in a S100A9i background, FIG. 12l). Taken together, these data show that the S100A9-ALDH1A 1 axis promotes the growth of EGFR-mutant lung cancer cells in the lung, albeit to a lesser extent than the brain.

PC9-derived BrM cells can grow in the brain and bone after injection into the arterial circulation (FIG. 1b and FIG. 8a). Therefore, we next tested whether the expression of S100A9 and ALDH1A1 is elevated in the PC9-BrM-derived bone lesions post-osimertinib treatment compared to vehicle-treated mice following tumor-cell injection, analogous to what we observed for brain lesions.

We analyzed the bone lesions at five months following tumor-cell injection, which is an intermediate timepoint before osimertinib effectively eliminates bone metastatic lesions in this model. In contrast to the brain, immunohistochemical analysis showed no significant increases in either S100A9 or ALDH1A1 expression in the bone metastatic lesions from the osimertinib-treated group (referred to as “Osi-residual tumor”) compared to bone metastatic lesions from the vehicle-treated group (FIG. 12m-r).

To confirm these findings, we isolated PC9 bone derivatives from osimertinib-treated mice injected with PC9-BrM cells (referred to as “PC9-bone-derivatives”) at 5 months following tumor-cell injection. We transduced the PC9-bone-derivatives with lentivirus encoding either Lenti-con, S100A9i, ALDH1A1i or S100A9i-ALDH1A1o/e (FIG. 12s). To evaluate the requirement of the S100A9-ALDH1A1 axis for growth in the bone, we directly implanted these transduced PC9-bone derivatives in the tibia bone of immunodeficient mice and evaluated the growth of these cells in the bone at endpoint (3 weeks post-injection, FIG. 12t).

No significant differences were observed in tumor growth in the bone between these groups as determined by quantitative bioluminescence imaging (FIG. 12u). These results suggest site-specific functions of the S100A9-ALDH1A1 axis, which promotes metastatic growth in the brain, to a lesser extent in the lung but not in the bone.

Osimertinib-Refractory Tumor Cells from Brain Metastases are Sensitive to Pan-RAR Inhibition.

The physiological functions of RA are primarily mediated through binding to two families of retinoid nuclear receptors, the RARs (RAR alpha, RAR beta and RAR gamma) and RXRs (RXR alpha, RXR beta and RXR gamma) that function as ligand-dependent transcription factors (43). RA-bound RAR/RXR heterodimers bind to target genes at RA-response elements (RAREs) to regulate their transcriptional activation (43). Since high expression of ALDH1A1 mediates S100A9-dependent brain metastasis, we hypothesized that activation of the RA response pathway enables Tr-BrM cells to grow in the brain in the presence of osimertinib.

To test the sensitivity of Tr-BrM cells to RAR pathway inhibition, we pharmacologically challenged PC9- and H1650-derived Tr-BrM cells with RAR pathway antagonists. Treatment of PC9- and H1650-Tr-BrM cells with retinal in serum-free media significantly induced expression of the RAR target gene STRA6 in vitro (FIG. 13a-b), indicating functional RA biosynthetic and response pathways. Interestingly, STRA6 expression was only modestly inhibited when treated with either the RAR-alpha antagonist BMS195614 (abbreviated as RAR-ai) or the RAR-gamma antagonist MM11253 (abbreviated as RAR-yi), but was dramatically reduced with the pan-RAR antagonist AGN-194310 (abbreviated as “pan-RAR-inh” (FIG. 13a-b).

In line with these observations, retinal-treated PC9- and H1650-Tr-BrM cells showed a striking dose-dependent cytotoxicity with pan-RAR antagonism compared to PC9- and H1650-BrM cells (FIG. 13c). Conversely, two other EGFR-mutant lung cancer cell lines, H1975 and HCC4006, that lack expression of S100A9 and ALDH1A1, did not show dose-dependent loss of viability with AGN-194310 (FIG. 13c-e).

To determine whether treatment with AGN-194310 inhibits brain metastasis in vivo, we injected PC9- and H1650-Tr-BrM cells into the arterial circulation of immunodeficient mice via intracardiac injection (FIG. 6a). We confirmed metastatic signal by bioluminescence imaging at 25 days and initiated a treatment study involving the administration five days per week of either 1) vehicle, 2) AGN-194310 (0.5 mg/kg body weight/day), 3) osimertinib (5 mg/kg body weight/day) or 4) AGN-194310 (0.5 mg/kg body weight/day) plus osimertinib (5 mg/kg body weight/day).

A striking reduction in brain metastasis was observed in mice treated with AGN-194310, compared to vehicle control and osimertinib alone groups, by bioluminescence imaging and histological analysis of the metastasis surface area with CK7 immunostaining (FIG. 6b-e and FIG. 13f-g). To complement our pharmacological inhibition studies, we generated PC9- and H1650-Tr-BrM cells expressing two independent sets of short hairpins (shRNA) targeting both RARa and RARy (FIG. 13h-k).

Consistent with the pharmacological studies (FIG. 6b-e), a striking reduction in brain metastasis was observed with both sets of shRNAs targeting RARa and RARy in both PC9- and H1650-derived cells, as determined by quantitative bioluminescence imaging and by histological analysis of metastasis surface area with CK7 immunostaining (FIG. 6f-j and FIG. 13l-o). We next asked whether AGN-194310 also impacts tumor growth in the bone or lung. Similar to our genetic suppression experiments (FIG. 12), AGN-194310 treatment modestly reduced lung (but not bone) lesions in the PC9- and H1650-derived models when directly implanted in the lung and bone, respectively (FIG. 13p-s). Our study therefore reveals a therapeutic vulnerability in osimertinib-refractory, brain-metastatic lung cancer cells that can be targeted by pan-RAR antagonism.

The Combination of Osimertinib with Pan-RAR Antagonism Reduces Residual Cancer Cells in the Brain.

Based on the existence of clonal heterogeneity in S100A9 expression in PC9-BrM cells prior to osimertinib treatment in the brain (FIG. 3), we asked whether treatment with the combination of osimertinib and AGN-194310 could reduce residual cancer cells in the brain. We injected unselected, treatment-naive, PC9- and H1650-BrM cells into the arterial circulation of immunodeficient mice via intracardiac injection (FIG. 7a). We initiated a prevention study at 5 days post tumor-cell injection involving the administration five days per week of either 1) vehicle, 2) AGN-194310 (0.5 mg/kg body weight/day), 3) osimertinib (5 mg/kg body weigh/day) or 4) AGN-194310 (0.5 mg/kg body weight/day) plus osimertinib (5 mg/kg body weigh/day).

At end point (7 weeks post tumor-cell injection), we harvested, sectioned and immunostained brain tissues with a CK7 antibody to identify cancer cells that were below the detection limit of bioluminescence imaging (FIG. 7b-c). Compared to the vehicle-treated mice, there was a significant reduction in the number of residual cancer cells in the brain in all treatment groups (FIG. 7b-c). Importantly, mice in the AGN-194310-plus-osimertinib group showed a significantly greater reduction in the residual disease burden in the brain compared to either osimertinib or AGN-194310 alone (FIG. 7b-c). These preclinical studies suggest that the combination of therapies (AGN-194310-plus-osimertinib) could prevent or delay the emergence of osimertinib-refractory disease in the brain of EGFR-mutant lung cancer patients.

It is to be understood that the compositions and method for treating EGFR-mutant lung cancer while avoiding metastatic relapse in the patient is not limited to the specific embodiments described above, but encompasses any and all embodiments within the scope of the generic language of the following claims enabled by the embodiments described herein, or otherwise shown in the drawings or described above in terms sufficient to enable one of ordinary skill in the art to make and use the claimed subject matter.

### REFERENCES

1. Sung H, Ferlay J, Siegel R L, Laversanne M, Soerjomataram I, Jemal A, et al. Global Cancer Statistics 2020: GLOBOCAN Estimates of Incidence and Mortality Worldwide for 36 Cancers in 185 Countries. C A Cancer J Clin 2021; 71 (3): 209-49 doi 10.3322/caac.21660.
2. Shi Y, Au J S, Thongprasert S, Srinivasan S, Tsai C M, Khoa M T, et al. A prospective, molecular epidemiology study of EGFR mutations in Asian patients with advanced non-small-cell lung cancer of adenocarcinoma histology (PIONEER). J Thorac Oncol 2014; 9 (2): 154-62 doi 10.1097/JT0.0000000000000033.
3. Midha A, Dearden S, McCormack R. EGFR mutation incidence in non-small-cell lung cancer of adenocarcinoma histology: a systematic review and global map by ethnicity (mutMapll). Am J Cancer Res 2015; 5 (9): 2892-911.
4. Xiaojing Du B Y, Quanlin An, Yehuda G. Assaraf, Xin Cao, and Jinglin Xia. Acquired resistance to third-generation EGFR-TKIs and emerging next-generation EGFR inhibitors. The Innovation 2021; 2 (2): 1-17.
5. Rosell R, Moran T, Queralt C, Porta R, Cardenal F, Camps C, et al. Screening for epidermal growth factor receptor mutations in lung cancer. N Engl J Med 2009; 361 (10): 958-67 doi 10.1056/NEJMoa0904554.
6. Lynch T J, Bell D W, Sardella R, Gurubhagavatula S, Okimoto R A, Brannigan B W, et al. Activating mutations in the epidermal growth factor receptor underlying responsiveness of non-small-cell lung cancer to gefitinib. N Engl J Med 2004; 350 (21): 2129-39 doi 10.1056/NEJMoa040938.
7. Sharma S V, Bell D W, Settleman J, Haber D A. Epidermal growth factor receptor mutations in lung cancer. Nat Rev Cancer 2007; 7 (3): 169-81 doi 10.1038/nrc2088.
8. Peters S, Mok T, Passaro A, Janne P A. The Promising Evolution of Targeted Therapeutic Strategies in Cancer. Cancer Discov 2021; 11 (4): 810-4 doi 10.1158/2159-8290.CD-21-0124.
9. Rotow J, Bivona T G. Understanding and targeting resistance mechanisms in NSCLC. Nat Rev Cancer 2017; 17 (11): 637-58 doi 10.1038/nrc.2017.84.
10. Paez J G, Janne P A, Lee J C, Tracy S, Greulich H, Gabriel S, et al. EGFR mutations in lung cancer: correlation with clinical response to gefitinib therapy. Science 2004; 304 (5676): 1497-500 doi 10.1126/science. 1099314.
11. Pao W, Miller V A, Politi K A, Riely G J, Somwar R, Zakowski M F, et al. Acquired resistance of lung adenocarcinomas to gefitinib or erlotinib is associated with a second mutation in the EGFR kinase domain. PLOS Med 2005; 2 (3): e73 doi 10.1371/journal.pmed.0020073.
12. Cross D A, Ashton S E, Ghiorghiu S, Eberlein C, Nebhan C A, Spitzler P J, et al. AZD9291, an irreversible EGFR TKI, overcomes T790M-mediated resistance to EGFR inhibitors in lung cancer. Cancer Discov 2014; 4 (9): 1046-61 doi 10.1158/2159-8290.CD-14-0337.
13. Soria J C, Ohe Y, Vansteenkiste J, Reungwetwattana T, Chewaskulyong B, Lee K H, et al. Osimertinib in Untreated EGFR-Mutated Advanced Non-Small-Cell Lung Cancer. N Engl J Med 2018; 378 (2): 113-25 doi 10.1056/NEJMoa1713137.
14. Ramalingam S S, Vansteenkiste J, Planchard D, Cho B C, Gray J E, Ohe Y, et al. Overall Survival with Osimertinib in Untreated, EGFR-Mutated Advanced NSCLC. N Engl J Med 2020; 382 (1): 41-50 doi 10.1056/NEJMoa1913662.
15. Piotrowska Z, Isozaki H, Lennerz J K, Gainor J F, Lennes I T, Zhu V W, et al. Landscape of Acquired Resistance to Osimertinib in EGFR-Mutant NSCLC and Clinical Validation of Combined EGFR and RET Inhibition with Osimertinib and BLU-667 for Acquired RET Fusion. Cancer Discov 2018; 8 (12): 1529-39 doi 10.1158/2159-8290.CD-18-1022.
16. Gregorc V, Lazzari C, Karachaliou N, Rosell R, Santarpia M. Osimertinib in untreated epidermal growth factor receptor (EGFR)-mutated advanced non-small cell lung cancer. Transl Lung Cancer Res 2018; 7 (Suppl 2): S165-S70 doi 10.21037/tlcr.2018.03.19.
17. Leonetti A, Sharma S, Minari R, Perego P, Giovannetti E, Tiseo M. Resistance mechanisms to osimertinib in EGFR-mutated non-small cell lung cancer. Br J Cancer 2019; 121 (9): 725-37 doi 10.1038/s41416-019-0573-8.
18. Ricordel C, Friboulet L, Facchinetti F, Soria J C. Molecular mechanisms of acquired resistance to third-generation EGFR-TKIs in EGFR T790M-mutant lung cancer. Ann Oncol 2018; 29 (suppl_1): 128-i37 doi 10.1093/annonc/mdx705.
19. Yu H A, Tian S K, Drilon A E, Borsu L, Riely G J, Arcila M E, et al. Acquired Resistance of EGFR-Mutant Lung Cancer to a T790M-Specific EGFR Inhibitor: Emergence of a Third Mutation (C797S) in the EGFR Tyrosine Kinase Domain. JAMA Oncol 2015; 1 (7): 982-4 doi 10.1001/jamaoncol.2015.1066.
20. Shin D Y, Na, 11, Kim C H, Park S, Baek H, Yang S H. EGFR mutation and brain metastasis in pulmonary adenocarcinomas. J Thorac Oncol 2014; 9 (2): 195-9 doi 10.1097/JTO.0000000000000069.
21. Rangachari D, Yamaguchi N, Vanderlaan P A, Felch E, Mahadevan A, Floyd S R, et al. Brain metastases in patients with EGFR-mutated or ALK-rearranged non-small-cell lung cancers. Lung Cancer 2015; 88 (1): 108-11 doi 10.1016/j.lungcan.2015.01.020.
22. Ge M, Zhuang Y, Zhou X, Huang R, Liang X, Zhan Q. High probability and frequency of EGFR mutations in non-small cell lung cancer with brain metastases. J Neurooncol 2017; 135 (2): 413-8 doi 10.1007/s11060-017-2590-x.
23. Heon S, Yeap B Y, Britt G J, Costa D B, Rabin M S, Jackman O M, et al. Development of central nervous system metastases in patients with advanced non-small cell lung cancer and somatic EGFR mutations treated with gefitinib or erlotinib. Clin Cancer Res 2010; 16 (23): 5873-82 doi 10.1158/1078-0432.CCR-10-1588.
24. Ballard P, Yates J W, Yang Z, Kim O W, Yang J C, Cantarini M, et al. Preclinical Comparison of Osimertinib with Other EGFR-TKIs in EGFR-Mutant NSCLC Brain Metastases Models, and Early Evidence of Clinical Brain Metastases Activity. Clin Cancer Res 2016; 22 (20): 5130-40 doi 10.1158/1078-0432.CCR-16-0399.
25. Reungwetwattana T, Nakagawa K, Cho B C, Cobo M, Cho E K, Bertolini A, et al. CNS Response to Osimertinib Versus Standard Epidermal Growth Factor Receptor Tyrosine Kinase Inhibitors in Patients With Untreated EGFR-Mutated Advanced Non-Small-Cell Lung Cancer. J Clin Oneel 2018: JCO2018783118 doi 10.1200/JCO.2018.78.3118.
26. Gilbert L A, Hemann M T. DNA damage-mediated induction of a chemoresistant niche. Cell 2010; 143 (3): 355-66 doi 10.1016/j.cell.2010.09.043.
27. Carlson P, Dasgupta A, Grzelak C A, Kim J, Barrett A, Coleman I M, et al. Targeting the perivascular niche sensitizes disseminated tumour cells to chemotherapy. Nat Cell Biol 2019; 21 (2): 238-50 doi 10.1038/s41556-018-0267-0.
28. Hobbs J A, May R, Tanousis K, McNeil! E, Mathies M, Gebhardt C, et al. Myeloid cell function in MRP-14 (S100A9) null mice. Mol Cell Biol 2003; 23 (7): 2564-76 doi 10.1128/MCB.23.7.2564-2576.2003.
29. Kawai H, Minamiya Y, Takahashi N. Prognostic impact of S100A9 overexpression in non-small cell lung cancer. Tumour Biol 2011; 32 (4): 641-6 doi 10.1007/s13277-011-0163-8.
30. Bos P D, Nguyen D X, Massague J. Modeling metastasis in the mouse. Curr Opin Pharmacol 2010; 10 (5): 571-7 doi 10.1016/j.coph.2010.06.003.
31. Nguyen D X, Chiang A C, Zhang X H, Kim J Y, Kris M G, Ladanyi M, et al. WNT/TCF signaling through LEF1 and HOXB9 mediates lung adenocarcinoma metastasis. Cell 2009; 138 (1): 51-62 doi 10.1016/j.cell.2009.04.030.
32. Mok T S, Wu Y L, Ahn M J, Garassino M C, Kim H R, Ramalingam S S, et al. Osimertinib or Platinum-Pemetrexed in EGFR T790M-Positive Lung Cancer. N Engl J Med 2017; 376 (7): 629-40 doi 10.1056/NEJMoa1612674.
33. Planchard D, Boyer M J, Lee J S, Dechaphunkul A, Cheema P K, Takahashi T, et al. Postprogression Outcomes for Osimertinib versus Standard-of-Care EGFR-TKI in Patients with Previously Untreated EGFR-mutated Advanced Non-Small Cell Lung Cancer. Clin Cancer Res 2019; 25 (7): 2058-63 doi 10.1158/1078-0432.CCR-18-3325.
34. Sos M L, Koker M, Weir B A, Heynck S, Rabinovsky R, Zander T, et al. PTEN loss contributes to erlotinib resistance in EGFR-mutant lung cancer by activation of Akt and EGFR. Cancer Res 2009; 69 (8): 3256-61 doi 10.1158/0008-5472.CAN-08-4055.
35. Wang G, Biswas A K, Ma W, Kandpal M, Coker C, Grandgenett P M, et al. Metastatic cancers promote cachexia through ZIP14 upregulation in skeletal muscle. Nat Med 2018; 24 (6): 770-81 doi 10.1038/s41591-018-0054-2.
36. Acharyya S, Oskarsson T, Vanharanta S, Malladi S, Kim J, Morris P G, et al. A CXCL1 paracrine network links cancer chemoresistance and metastasis. Cell 2012; 150 (1): 165-78 doi 10.1016/j.cell.2012.04.042.
37. Bresnick A R, Weber D J, Zimmer D B. S100 proteins in cancer. Nat Rev Cancer 2015; 15 (2): 96-109 doi 10.1038/nrc3893.
38. Raudvere U, Kolberg L, Kuzmin I, Arak T, Adler P, Peterson H, et al. g: Profiler: a web server for functional enrichment analysis and conversions of gene lists (2019 update). Nucleic Acids Res 2019; 47 (W1): W191-W8 doi 10.1093/nar/gkz369.
39. Achrol A S, Rennert R C, Anders C, Soffietti R, Ahluwalia M S, Nayak L, et al. Brain metastases. Nat Rev Dis Primers 2019; 5 (1): 5 doi 10.1038/s41572-018-0055-y.
40. Winkler F. The brain metastatic niche. J Mol Med (Berl) 2015; 93 (11): 1213-20 doi 10.1007/s00109-015-1357-0.
41. Valiente M, Obenauf A C, Jin X, Chen Q, Zhang X H, Lee D J, et al. Serpins promote cancer cell survival and vascular co-option in brain metastasis. Cell 2014; 156 (5): 1002-16 doi 10.1016/j.cell.2014.01.040.
42. Chambon P. A decade of molecular biology of retinoic acid receptors. FASEB J 1996; 10 (9): 940-54.
43. Tang X H, Gudas L J. Retinoids, retinoic acid receptors, and cancer. Annu Rev Pathol 2011; 6:345-64 doi 10.1146/annurev-pathol-011110-130303.
44. Colclough N, Chen K, Johnstrom P, Strittmatter N, Yan Y, Wrigley G L, et al. Preclinical Comparison of the Blood-brain barrier Permeability of Osimertinib with Other EGFR TKIs. Clin Cancer Res 2021; 27 (1): 189-201 doi 10.1158/1078-0432.CCR-19-1871.
45. Schonthaler H B, Guinea-Viniegra J, Wculek S K, Ruppen I, Ximenez-Embun P, Guio-Carrion A, et al. S100A8-S100A9 protein complex mediates psoriasis by regulating the expression of complement factor C3. Immunity 2013; 39 (6): 1171-81 doi 10.1016/j.immuni.2013.11.011.
46. Song R, Struhl K. S100A8/S100A9 cytokine acts as a transcriptional coactivator during breast cellular transformation. Sci Adv 2021; 7 (1) doi 10.1126/sciadv.abe5357.
47. Li Y, Wongsiriroj N, Blaner W S. The multifaceted nature of retinoid transport and metabolism. Hepatobiliary Surg Nutr 2014; 3 (3): 126-39 doi 10.3978/j.issn.2304-3881.2014.05.04.
48. Kelly M, von Lintig J. STRA6: role in cellular retinal uptake and efflux. Hepatobiliary Surg Nutr 2015; 4 (4): 229-42 doi 10.3978/j.issn.2304-3881.2015.01.12.
49. Hiratsuka S, Watanabe A, Aburatani H, Maru Y. Tumour-mediated upregulation of chemoattractants and recruitment of myeloid cells predetermines lung metastasis. Nat Cell Biol 2006; 8 (12): 1369-75 doi 10.1038/ncb1507.
50. Rafii S, Lyden D. S100 chemokines mediate bookmarking of premetastatic niches. Nat Cell Biol 2006; 8 (12): 1321-3 doi 10.1038/ncb1206-1321.
51. Cheng P, Corzo C A, Luetteke N, Yu B, Nagaraj S, Bui M M, et al. Inhibition of dendritic cell differentiation and accumulation of myeloid-derived suppressor cells in cancer is regulated by S100A9 protein. J Exp Med 2008; 205 (10): 2235-49 doi 10.1084/jem.20080132.
52. Burke M, Choksawangkarn W, Edwards N, Ostrand-Rosenberg S, Fenselau C. Exosomes from myeloid-derived suppressor cells carry biologically active proteins. J Proteome Res 2014; 13 (2): 836-43 doi 10.1021/pr400879c.
53. Wortzel I, Dror S, Kenific C M, Lyden D. Exosome-Mediated Metastasis: Communication from a Distance. Dev Cell 2019; 49 (3): 347-60 doi 10.1016/j.devcel.2019.04.011.
54. Ichikawa M, Williams R, Wang L, Vogl T, Srikrishna G. S100A8/A9 activate key genes and pathways in colon tumor progression. Mol Cancer Res 2011; 9 (2): 133-48 doi 10.1158/1541-7786.MCR-10-0394.
55. Johnson A T, Wang L, Standeven A M, Escobar M, Chandraratna R A. Synthesis and biological activity of high-affinity retinoic acid receptor antagonists. Bioorg Med Chem 1999; 7 (7): 1321-38 doi 10.1016/s0968-0896 (99) 00055-3.
56. Hammond L A, Van Krinks C H, Durham J, Tomkins S E, Burnett R D, Jones E L, et al. Antagonists of retinoic acid receptors (RARs) are potent growth inhibitors of prostate carcinoma cells. Br J Cancer 2001; 85 (3): 453-62 doi 10.1054/bjoc.2001.1939.
57. Petrie K, Urban-Wojciuk Z, Sbirkov Y, Graham A, Hamann A, Brown G. Retinoic acid receptor gamma is a therapeutically targetable driver of growth and survival in prostate cancer. Cancer Rep (Hoboken) 2020; 3 (6): e1284 doi 10.1002/cnr2.1284.
58. Maloney C, Edelman M C, Kallis M P, Soffer S Z, Symons M, Steinberg B M. Intratibial Injection Causes Direct Pulmonary Seeding of Osteosarcoma Cells and Is Not a Spontaneous Model of Metastasis: A Mouse Osteosarcoma Model. Clin Orthop Relat Res 2018; 476 (7): 1514-22 doi 10.1007/s11999.0000000000000291.
59. Safari R, Meuwissen R. Practical use of advanced mouse models for lung cancer. Methods Mol Biol 2015; 1267:93-124 doi 10.1007/978-1-4939-2297-0_5.
60. Gilbert L A, Horlbeck M A, Adamson B, Villalta J E, Chen Y, Whitehead E H, et al. Genome-Scale CRISPR-Mediated Control of Gene Repression and Activation. Cell 2014; 159 (3): 647-61 doi 10.1016/j.cell.2014.09.029.
61. Sanjana N E, Shalem O, Zhang F. Improved vectors and genome-wide libraries for CRISPR screening. Nat Methods 2014; 11 (8): 783-4 doi 10.1038/nmeth.3047.
62. Hayer A, Shao L, Chung M, Joubert L M, Yang H W, Tsai F C, et al. Engulfed cadherin fingers are polarized junctional structures between collectively migrating endothelial cells. Nat Cell Biol 2016; 18 (12): 1311-23 doi 10.1038/ncb3438.
63. Biswas A K, Han S, Tai Y, Ma W, Coker C, Quinn S A, Shakri A R, Zhong T J, Scholze H, Lagos G G, Mela A, Manova-Todorova K, de Stanchina E, Ferrando A A, Mendelsohn C, Canoll P, Yu H A, Paik P K, Kris M G, Massague J, Acharya S. Targeting S100A9-ALDH1A1-retinoic acid signaling to suppress brain relapse in EGFR-mutant lung cancer. Cancer Discovery 2022; 12 (4): 1002-1021 10.1158/2159-8290.CD-21-0910.

What is claimed is:

1. A method of treating a patient with epidermal growth factor receptor (EGFR)-mutant lung cancer, comprising administering to the patient an effective amount of at least one EGFR tyrosine kinase inhibitor (TKI) while avoiding metastatic relapses in the patient by targeting S100A9-ALDH1A1-retinoic acid signaling.

2. The method of treating a patient with EGFR-mutant lung cancer as recited in claim 1, wherein the method inhibits S100A9, ALDH1A1, or retinoic acid receptors.

3. The method of treating a patient with EGFR-mutant lung cancer as recited in claim 2, wherein the at least one EGFR TKI comprises an osimertinib formulation.

4. The method of treating a patient with EGFR-mutant lung cancer as recited in claim 3, wherein the method further includes administration of RAR antagonists and helps avoid increased brain metastasis.

5. The method of treating a patient with EGFR-mutant lung cancer as recited in claim 4, wherein the method includes administration of AGN-194310.

6. A pharmaceutical composition for treating a patient with EGFR-mutant lung cancer while avoiding metastatic relapses in the patient, the composition comprising at least one EGFR tyrosine kinase inhibitor (TKI) that targets S100A9-ALDH1A1-retinoic acid signaling and avoids metastatic relapses in the patient.

7. The pharmaceutical composition as recited in claim 6, wherein the composition inhibits S100A9, ALDH1A1, or retinoic acid receptors.

8. The pharmaceutical composition as recited in claim 6, wherein the composition comprises osimertinib.

9. The composition as recited in claim 7, wherein the composition helps avoid increased brain metastasis.

10. The composition as recited in claim 8, wherein the composition further comprises AGN-194310.

11. The composition as recited in claim 10, wherein the composition is administered at one time.

12. The composition as recited in claim 10, wherein the composition is administered in separate components.

13. A method of detecting osimertinib resistance in a patient with cancer, the method comprising determining the level of S100A9 expressed by cancer cells in the patient.

14. A method of treating a cancer patient with osimertinib resistance, the method comprising the steps of
(a) detecting osimertinib resistance as recited in claim 13, and
(b) administering an RAR inhibitor to the patient.

15. The method as recited in claim 14, wherein the RAR inhibitor is AGN194310.

16. The method as recited in claim 14, wherein the RAR inhibitor is co-administered with osimertinib to the cancer patient.

17. The method as recited in claim 15, wherein the RAR inhibitor is co-administered with osimertinib to the cancer patient.

18. The method as recited in claim 13, wherein the method further comprises comparing S100A9 levels expressed by the cancer cells both before and after administration of an RAR inhibitor.

19. The method as recited in claim 18, wherein the RAR inhibitor is AGN194310.
